(19) United States
(12) Patent Application Publication
(10) Pub. No.: US 2026/0055347 A1
(43) Pub. Date: Feb. 26, 2026

(54) AMYLASE VARIANTS

(71) Applicant: BASF SE, Ludwigshafen am Rhein (DE)

(72) Inventors: Stefan JENEWEIN, Ludwigshafen (DE); Cristina POP, San Diego, CA (US); Cindy HOANG, San Diego, CA (US); Janina BERNDT, Ludwigshafen (DE); Jesper NIELSEN, San Diego, CA (US); Jonathan D. LYON, sAN dIEGO, CA (US); Amanda Rae LOGUE, San Diego, CA (US); Michael Wulff RISOER, San Diego, CA (US); Katie KLINE, San Diego, CA (US)

(21) Appl. No.: 19/103,190

(22) PCT Filed: Jul. 31, 2023
(86) PCT No.: PCT/EP2023/071183

§ 371 Date: Feb. 11, 2025

(30) Foreign Application Priority Data
Aug. 11, 2022 (EP) 22189952.9

(51) Int. Cl. C11D 3/386; C12N 9/26

(52) U.S. Cl. CPC C11D 3/38618; C12N 9/2414; C12Y 302/01001

(57) ABSTRACT

In the present invention new amylase enzymes are provided. More specifically, genetically engineered amylase enzymes, compositions comprising the enzymes, and methods of making and using the enzymes or compositions comprising the enzymes are provided.

### FIELD OF THE INVENTION

In the present invention new amylase enzymes are provided. More specifically, genetically engineered amylase enzymes, compositions comprising the enzymes, and methods of making and using the enzymes or compositions comprising the enzymes are provided.

### BACKGROUND OF THE INVENTION

Enzymes are increasingly used in various application as sustainable alternative to petrochemistry. Enzymes are biodegradable and can be catalytically active already at lower temperatures, which results in reduction of energy consumption. In particular, in the detergent industry enzymes are implemented in washing formulations to improve cleaning efficiency and reducing energy consumption in a washing step.

Amylases are enzymes capable of hydrolyzing starch. Thus, amylases have been employed in the removal of starch stains and have been added to detergent compositions for this purpose. In detergent applications the amylases shall be stable at elevated temperatures and/or within the denaturing conditions of the detergents and the wash liquor. Thus, the need exists for new amylase enzymes, which meet these requirements.

### BRIEF SUMMARY OF THE INVENTION

The present invention is directed to an amylase variant of a parent amylase, wherein said variant comprises
(i) an amino acid substitution at position 4 according to the numbering of SEQ ID NO: 3,
(ii) an amino acid substitution at one or more, preferably two or more, amino acid positions corresponding to amino acid positions selected from the group consisting of 25, 176, 186, 251, 405, and 482 according to the numbering of SEQ ID NO: 3, and
(iii) at least 60%, preferably at least 91% identity, but less than 100% sequence identity with SEQ ID NO: 1, 3, 4, or in any of SEQ ID NO: 15-41, preferably of SEQ ID NO: 1 or SEQ ID NO: 3, preferably SEQ ID NO: 1, preferably wherein the parent amylase of the amylase variant is an amylase according to SEQ ID NO: 1 or SEQ ID NO: 3, preferably SEQ ID NO: 1.

In one embodiment, the amylase variant of the present invention comprises
(i) the amino acid substitution X4G according to the numbering of SEQ ID NO: 3,
(ii) one or more, two or more, three or more, four or more, five or more, or all of the amino acid substitutions selected from the group consisting of X25H, X176K, X186E, X251E, X405M, and X482W according to the numbering of SEQ ID NO: 3, and
(iii) at least 60%, at least 65%, at least 70%, at least 75%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% sequence identity with SEQ ID NO: 1, 3, 4, or with any of SEQ ID NO: 15-41, preferably with SEQ ID NO: 1 or SEQ ID NO: 3, preferably SEQ ID NO: 1.

In another embodiment, the present invention is directed to a polynucleotide encoding the amylase variant.

Moreover, the present invention is directed to a composition comprising the amylase variant. Preferably, the composition comprising the amylase variant is a detergent composition. The present invention is further directed to a method of making the amylase variant and methods of using the amylase variant, in particular for cleaning an object.

### DETAILED DESCRIPTION OF THE INVENTION

The present invention may be understood more readily by reference to the following detailed description of the embodiments of the invention and the examples included herein.

Although the present invention will be described with respect to particular embodiments, this description is not to be construed in a limiting sense.

### Definitions

Unless otherwise noted, the terms used herein are to be understood according to conventional usage by those of ordinary skill in the relevant art.

Before describing in detail exemplary embodiments of the present invention, definitions important for understanding the present invention are given. Unless stated otherwise or apparent from the nature of the definition, the definitions apply to all compounds, methods and uses described herein.

As used in this specification and in the appended claims, the singular forms of “a” and “an” also include the respective plurals unless the context clearly dictates otherwise.

In the context of the present invention, the terms “about” and “approximately” denote an interval of accuracy that a person skilled in the art will understand to still ensure the technical effect of the feature in question. The term typically indicates a deviation from the indicated numerical value of +20%, preferably +15%, more preferably +10%, and even more preferably +5%. Furthermore, the terms “first”, “second”, “third” or “(a)”, “(b)”, “(c)”, “(d)” etc. and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. In case the terms “first”, “second”, “third” or “(a)”, “(b)”, “(c)”, “(d)”, “i”, “ii” etc. re-late to steps of a method or use or assay there is no time or time interval coherence between the steps, i.e. the steps may be carried out simultaneously or there may be time intervals of seconds, minutes, hours, days, weeks, months or even years between such steps, unless otherwise indicated in the application as set forth herein above or below.

Throughout this application, various publications are referenced. The disclosures of all of these publications and those references cited within those publications in their entireties are hereby incorporated by reference into this application in order to more fully describe the state of the art to which this invention pertains.

It is to be understood that the term “comprising” is not limiting. For the purposes of the present invention the term “consisting of” is considered to be a preferred embodiment of the term “comprising”. If hereinafter a group is defined to comprise at least a certain number of members, this is meant to also encompass a group which consists of these members only. “Variant” enzymes differ from “parent” enzymes by certain amino acid alterations, preferably amino acid substitutions at one or more amino acid positions.

In describing the variants of the present invention, the abbreviations for single amino acids are used according to the accepted IUPAC single letter or three letter amino acid abbreviation. “Amino acid alteration” as used herein refers to amino acid substitution, deletion, or insertion. “Substitutions” are described by providing the original amino acid followed by the number of the position within the amino acid sequence, followed by the amino acid, which substitutes the original amino acid. For example, the substitution of histidine at position 120 with alanine is designated as “His120Ala” or “H120A”. Substitutions can also be described by merely naming the resulting amino acid in the variant without specifying the amino acid of the parent at this position, e.g., “X120A” or “120A” or “Xaa120Ala” or “120Ala”.

“Deletions” are described by providing the original amino acid followed by the number of the position within the amino acid sequence, followed by *. Accordingly, the deletion of glycine at position 150 is designated as “Gly150*” or G150*”. Alternatively, deletions are indicated by, e.g., “deletion of D183 and G184”.

“Insertions” are described by providing the original amino acid followed by the number of the position within the amino acid sequence, followed by the original amino acid and the additional amino acid. For example, an insertion at position 180 of lysine next to glycine is designated as “Gly180GlyLys” or “G180GK”. When more than one amino acid residue is inserted, such as, e.g., a Lys and an Ala after Gly180 this may be indicated as: “Gly180GlyLysAla” or “G195GKA”.

In cases where a substitution and an insertion occur at the same position, this may be indicated as “S99SD+S99A” or in short “S99AD”. Variants comprising multiple alterations are separated by “+”, e.g., “Arg170Tyr+Gly195Glu”, “R170Y+G195E” or “X170Y+X195E” representing a substitution of arginine and glycine at positions 170 and 195 with tyrosine and glutamic acid, respectively. Alternatively, multiple alterations may be separated by space or a comma, e.g.,

“R170Y G195E” or “R170Y, G195E” respectively. Where different alternative alterations can be introduced at a position, the different alterations are separated by a comma, e.g., “Arg170Tyr, Glu” and “R170T, E”, respectively, represents a substitution of arginine at position 170 with tyrosine or glutamic acid. Alternative substitutions at a particular position can also be indicated as “X120A,G,H”, “120A,G,H”, “X120A/G/H”, or “120A/G/H”. Alternatively, different alterations or op-tional substitutions may be indicated in brackets, e.g., “Arg170 [Tyr, Gly]” or “Arg170 {Tyr, Gly}” or in short “R170 [Y, G]” or “R170 {Y, G}”.

The term “native” (or naturally or wildtype or endogenous) cell or organism or polynucleotide or polypeptide refers to the cell or organism or polynucleotide or polypeptide as found in nature (i.e., without there being any human intervention).

The term “heterologous” (or exogenous or foreign or recombinant or non-native or non-naturally) polypeptide is defined herein as a polypeptide that is not native to the host cell, a polypeptide native to the host cell in which structural modifications, e.g., deletions, substitutions, and/or insertions, have been made by recombinant DNA techniques to alter the native polypeptide, or a polypeptide native to the host cell whose expression is quantitatively altered or whose expression is directed from a genomic location different from the native host cell as a result of manipulation of the DNA of the host cell by recombinant DNA techniques, e.g., a stronger promoter. Similarly, the term “heterologous” (or exogenous or foreign or recombinant or non-native or non-naturally) polynucleotide refers to a polynucleotide that is not native to the host cell, a polynucleotide native to the host cell in which structural modifications, e.g., deletions, substitutions, and/or insertions, have been made by recombinant DNA techniques to alter the native polynucleotide, or a polynucleotide native to the host cell whose expression is quantitatively altered as a result of manipulation of the regulatory elements of the polynucleotide by recombinant DNA techniques, e.g., a stronger promoter, or a polynucleotide native to the host cell, but integrated not within its natural genetic environment as a result of genetic manipulation by recombinant DNA techniques. With respect to the relation between two or more polynucleotide sequences or the relation between two or more amino acid sequences, the term “heterologous” is used to characterize that the two or more polynucleotide sequences or two or more amino acid sequences are naturally not occurring in the specific combination with each other.

For the purpose of the invention, “recombinant” (or transgenic) with regards to a cell or an organism means that the cell or organism contains a heterologous polynucleotide, which is introduced by man using gene technology. With regards to a polynucleotide “recombinant” includes all constructs produced by using gene technology/recombinant DNA techniques in which either
(a) the sequence of the polynucleotide or a part thereof, or
(b) one or more genetic control sequences, which are operably linked to the polynucleotide, including but not limited to a promoter, or
(c) both a) and b) are not located in their wildtype genetic environment or have been modified by man.

A “synthetic” compound is obtained by in vitro chemical and/or enzymatic synthesis.

Variant polynucleotide and variant polypeptide sequences may be defined by their sequence identity when compared to another sequence. Sequence identity usually is provided as “% sequence identity” or “% identity”. For calculation of sequence identities, in a first step a sequence alignment is produced. According to this invention, a pairwise global alignment is produced, meaning that two sequences are aligned over their complete length, which is usually produced by using a mathematical approach, called alignment algorithm.

According to the invention, the alignment is generated by using the algorithm of Needleman and Wunsch (J. Mol. Biol. (1979) 48, p. 443-453). Preferably, the program “NEEDLE” (The European Molecular Biology Open Software Suite (EMBOSS)) is used for the purposes of the current invention, with using the programs default parameter (polynucleotides: gap open=10.0, gap extend=0.5 and matrix=EDNAFULL; polypeptides: gap open=10.0, gap extend=0.5 and matrix=EBLOSUM62). After aligning two sequences, in a second step, an identity value is determined from the alignment produced. For this purpose, the %-identity is calculated by dividing the number of identical residues by the length of the alignment region which is showing the respective sequence of the present invention over its complete length multiplied with 100: %-identity=(identical residues/length of the alignment region which is showing the respective sequence of the present invention over its complete length)*100.

For calculating the percent identity of two nucleic acid sequences the same applies as for the calculation of percent identity of two amino acid sequences with some specifications. For nucleic acid sequences encoding for a protein the pairwise alignment shall be made over the complete length of the coding region of the sequence of this invention from start to stop codon excluding introns. Introns present in the other sequence, to which the sequence of this invention is compared, shall also be removed for the pairwise alignment. After aligning two sequences, in a second step, an identity value is determined from the alignment produced. Percent identity is calculated by %−identity=(identical residues/length of the alignment region which is showing the sequence of the invention from start to stop codon excluding introns over its complete length)*100.

Moreover, the preferred alignment program for nucleic acid sequences implementing the Needleman and Wunsch algorithm (J. Mol. Biol. (1979) 48, p. 443-453) is “NEEDLE” (The European Molecular Biology Open Software Suite (EMBOSS)) with the programs default parameters (gapopen=10.0, gapextend=0.5 and matrix=EDNAFULL).

Variant polypeptides may also be defined by their sequence similarity when compared to another sequence. Sequence similarity usually is provided as “% sequence similarity” or “%-similarity”. % sequence similarity takes into account that defined sets of amino acids share similar properties, e.g. by their size, by their hydrophobicity, by their charge, or by other characteristics. Herein, the exchange of one amino acid with a similar amino acid may be called “conservative mutation”. Similar amino acids according to the invention are defined as follows, which shall also apply for determination of %-similarity according to this invention, which is also in accordance with the BLOSUM62 matrix as for example used by program “NEEDLE”, which is one of the most used amino acids similarity matrix for database searching and sequence alignments:
Amino acid A is similar to amino acids S
Amino acid D is similar to amino acids E; N
Amino acid E is similar to amino acids D; K; Q
Amino acid F is similar to amino acids W; Y
Amino acid H is similar to amino acids N; Y
Amino acid I is similar to amino acids L; M; V
Amino acid K is similar to amino acids E; Q; R
Amino acid L is similar to amino acids I; M; V
Amino acid M is similar to amino acids I; L; V
Amino acid N is similar to amino acids D; H; S
Amino acid Q is similar to amino acids E; K; R
Amino acid R is similar to amino acids K; Q
Amino acid S is similar to amino acids A; N; T
Amino acid T is similar to amino acids S
Amino acid V is similar to amino acids I; L; M
Amino acid W is similar to amino acids F; Y
Amino acid Y is similar to amino acids F; H; W

For calculation of sequence similarity, in a first step a sequence alignment is produced as described above. After aligning two sequences, in a second step, a similarity value is determined from the alignment produced. For this purpose, the %-similarity is calculated by dividing the number of identical residues plus the number of similar residues by the length of the alignment region which is showing the sequence of the invention over its complete length multiplied with 100: %-similarity= [(identical residues+similar residues)/length of the alignment region which is showing the sequence of the invention over its complete length]*100.

For nucleic acids, similar sequences can also be determined by hybridization using respective stringency conditions. The term “high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 2×SSC, 0.2% SDS at 65° C. The term “very high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 2×SSC, 0.2% SDS at 70° C.

A “fragment” or “subsequence” as used herein are a portion of a polynucleotide or an amino acid sequence. The term “functional fragment” refers to any nucleic acid or amino acid sequence which comprises merely a part of the full-length amino acid sequence, respectively, but still has the same or similar activity and/or function. Preferably, the functional fragment is at least 75%, at least 76%, at least 77%, at least 78%, at least 79%, at least 80% identical, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 99%, or at least 99.5% identical to the original full length amino acid sequence. The functional fragment comprises consecutive nucleotides or amino acids compared to the original nucleic acid or original amino acid sequence, respectively.

“Genetic construct” or “expression cassette” as used herein, is a nucleic acid molecule composed of at least one sequence of interest to be expressed, operably linked to one or more control sequences (at least to a promoter) as described herein.

The term “vector” as used herein comprises any kind of construct suitable to carry foreign polynucleotide sequences for transfer to another cell, or for stable or transient expression within a given cell. The term “vector” as used herein encompasses any kind of cloning vehicles, such as but not limited to plasmids, phagemids, viral vectors (e.g., phages), bacteriophage, baculoviruses, cosmids, fosmids, artificial chromosomes, and any other vectors specific for specific hosts of interest. Foreign polynucleotide sequences usually comprise a coding sequence which may be referred to herein as “gene of interest”. The gene of interest may comprise introns and exons, depending on the kind of origin or destination of host cell.

The term “introduction of a polynucleotide” or “transformation of a polynucleotide” as referred to herein encompasses the transfer of an exogenous polynucleotide into a host cell, irrespective of the method used for transfer. That is, the term “transformation of a polynucleotide” as used herein is independent from vector, shuttle system, or host cell, and it not only relates to the polynucleotide transfer method of transformation as known in the art (cf., for example, Sambrook, J. et al. (1989) Molecular Cloning: A Laboratory Manual, 2nd Ed., Cold Spring Harbor Laboratory Press, Cold Spring Harbor, NY), but it encompasses any further kind polynucleotide transfer methods such as, but not limited to, transduction or transfection.

A polynucleotide encoding a polypeptide may be “expressed”. The term “expression” or “gene expression” means the transcription of a gene or genes or genetic construct into structural RNA (e.g., rRNA, tRNA) or mRNA with or without subsequent translation of the latter into a protein. The process includes transcription of DNA and processing of the resulting mRNA product. The term “purification” or “purifying” refers to a process in which at least one component, e.g., a protein of interest, is separated from at least another component, e.g., a particulate matter of a fermentation broth, and transferred into a different compartment or phase, wherein the different compartments or phases do not necessarily need to be separated by a physical barrier. Examples of such different compartments are two compartments separated by a filtration membrane or cloth, i.e., filtrate and retentate; examples of such different phases are pellet and supernatant or cake and filtrate, respectively. The resulting solution after purifying the enzyme of interest from the fermentation broth is called herein “purified enzyme solution”.

“Protein formulation” (or “enzyme preparation”), e.g., “protein variant formulation”, means any non-complex formulation comprising a small number of ingredients, wherein the ingredients serve the purpose of stabilizing the proteins comprised in the protein formulation and/or the sta-bilization of the protein formulation itself. Preferably, the non-complex protein formulation comprises the protein in higher concentrations than the complex formulation, e.g., than a detergent composition. Thus, preferably the non-complex protein formulation is a concentrated protein variant formulation. Preferably, non-complex protein formulations comprise 2 to 120 mg/g active enzyme, wherein complex formulations, like detergent compositions, comprise 0.002 to 6 mg/g active enzyme.

“Enzyme properties” include, but are not limited to catalytic activity, substrate/cofactor specificity, product specificity, stability in the course of time, thermostability, pH stability, and chemical stability. “Enzymatic activity” or “catalytic activity” means the catalytic effect exerted by an enzyme, expressed as units per milligram of enzyme (specific activity) or molecules of substrate transformed per minute per molecule of enzyme (molecular activity). Enzymatic activity can be specified by the enzymes actual function, e.g., proteases exerting proteolytic activity by catalyzing hydrolytic cleavage of peptide bonds, lipases exerting lipolytic activity by hydrolytic cleavage of ester bonds, amylases activity involves hydrolysis of glycosidic linkages in polysaccharides, etc.

The term “enzyme stability” according to the current invention relates to the retention of enzymatic activity as a function of time during storage or operation. Retention of enzymatic activity as a function of time during storage is called “storage stability” and is preferred within the context of the invention.

To determine and quantify changes in catalytic activity of enzymes stored or used under certain conditions over time, the “initial enzymatic activity” is measured under defined conditions at time zero (100%) and at a certain point in time later (x %). By comparison of the values measured, a potential loss of enzymatic activity can be determined in its extent. The extent of enzymatic activity loss determines an enzyme's stability or non-stability.

“Enzyme inhibitors” as used herein are compounds that slow down or halt enzymatic activity. Enzyme inhibitors frequently also stabilize the enzyme in its three-dimensional structure. Hence, enzyme inhibitors usually also act as “enzyme stabilizers”. “pH stability” refers to the ability of an enzyme to exert enzymatic activity after exposure to certain pH value.

The terms “thermal stability”, “thermostability” or “temperature-dependent activity” refer to the ability of an enzyme to exert catalytic activity or wash performance after exposure to elevated temperatures, preferably, at a temperature of 40° C. for 28 days, preferably 56 days, preferably in a detergent composition (preferably, in model ES1-C detergent), or at 92° C. for at least 10 min.

The terms “detergent stability” or “stability under storage in a detergent composition” refer to the ability of an enzyme to exert catalytic activity or wash performance after storage in a detergent composition, preferably, at a temperature of 40° C. or 50° C. for 28 days, preferably 56 days, in a detergent composition (preferably, in model ES1-C detergent).

“Improvement Factor” (IF) is the degree of improvement of an enzyme variant in a certain property, preferably over the respective parent enzyme. An improvement of the enzyme variant, preferably over the respective parent enzyme, is characterized by an Improvement Factor (IF) of >1.0. The improvement factor can alternatively be expressed in percentages, e.g., and IF of 1.1 equals 110%.

As used herein, “wash performance” (also called herein “cleaning performance”) of an enzyme refers to the contribution of the enzyme to the cleaning performance of a detergent composition, i.e. the cleaning performance added to the detergent composition by the performance of the enzyme. The term “wash performance” is used herein similarly for laundry and hard surface cleaning. Wash performance is compared under relevant washing conditions. The term “relevant washing conditions” is used herein to indicate the conditions, particularly washing temperature, time, washing mechanics, sud concentration, type of detergent and water hardness, actually used in households in a detergent market segment. The term “improved wash performance” is used to indicate that a better end result is obtained in stain removal under relevant washing conditions, or that less enzyme, on weight basis, is needed to obtain the same end result relative to the corresponding control conditions.

As used herein, the term “specific performance” refers to the cleaning and removal of specific stains or soils per unit of active enzyme. In some embodiments, the specific performance is determined using stains or soils such as egg, egg yolk, milk, grass, minced meat blood, chocolate sauce, baby food, sebum, etc.

“Detergent composition”, “detergent formulation”, or “detergent” means compositions designated for cleaning soiled material. Detergent compositions according to the invention include detergent compositions for different applications such as laundry and hard surface cleaning. The term “detergent component” is defined herein to mean a type of chemical, which can be used in detergent compositions. A typical detergent component is a surfactant. “Surfactant” (synonymously used herein with “surface active agent”) means an organic chemical that, when added to a liquid, changes the properties of that liquid at an interface. According to its ionic charge, a surfactant is called non-ionic, anionic, cationic, or amphoteric. The term “effective amount of a detergent component” includes amounts of certain components to provide effective stain removal and/or effective cleaning conditions (e.g. pH, temperature, water hardness), amounts of certain components to effectively provide optical benefits (e.g. optical brightening, dye transfer inhibition, color care), and amounts of certain components to effectively aid the processing (maintain physical characteristics during processing, storage and use; e.g. rheology modifiers, hydrotropes, desiccants). The term “laundry” or “laundering” relates to both household laundering and industrial laundering and means the process of treating textiles and/or fabrics with a solution containing a detergent composition of the present invention. The laundering process may be carried out by using technical devices such as a household or an industrial washing machine. Alternatively, the laundering process may be done by hand.

The term “textile” means any textile material including yarns (thread made of natural or synthetic fibers used for knitting or weaving), yarn intermediates, fibers, non-woven materials, natural materials, synthetic materials, as well as fabrics made of these materials such as garments, cloths and other articles. The terms “fabric” (a textile made by weaving, knitting or felting fibers) or “garment” (any article of clothing made of textile) as used herein, mean to include the broader term textile as well.

The term “fibers” includes natural fibers, synthetic fibers, and mixtures thereof. Examples of natural fibers are of plant (such as flax, jute and cotton) or animal origin, comprising proteins like collagen, keratin and fibroin (e.g. silk, sheep wool, angora, mohair, cashmere). Examples for fibers of synthetic origin are polyurethane fibers such as Spandex® or Lycra®, polyester fibers, polyolefins such as elastofin, or polyamide fibers such as nylon. Fibers may be single fibers or parts of textiles such as knitwear, woven or non-woven fabrics.

The term “hard surface cleaning” relates to both household hard surface cleaning and industrial hard surface cleaning and means the process of treating hard surfaces with a solution containing a detergent composition of the present invention. Hard surfaces may include any hard surfaces in the household or industry, such as floors, furnishing, walls, sanitary ceramics, glass, metallic surfaces including medical devices, cutlery, and dishes. A particular form of hard surface cleaning is dishwashing, manual dish washing (MDW) or automatic dishwashing (ADW). The term “dish wash” refers to all forms of washing dishes, e.g. by hand or automatic dish wash. Washing dishes includes, but is not limited to, the cleaning of all forms of crockery such as plates, cups, glasses, bowls, all forms of cutlery such as spoons, knives, forks and serving utensils as well as ceramics, plastics such as melamine, metals, china, glass and acrylics.

Cleaning performance is evaluated under relevant cleaning conditions. The term “relevant cleaning conditions” herein refers to the conditions, particularly cleaning temperature, time, cleaning mechanics, suds concentration, type of detergent and water hardness, actually used in laundry machines, automatic dish washers or in manual cleaning processes.

The term “medical device cleaning” refers to the cleaning step in reprocessing reusable medical devices. Medical device cleaning methods can be divided into two categories, manual and me-chanical/automated cleaning methods. Manual cleaning is used when mechanical units are not available or medical devices to be cleaned are too fragile or difficult to clean with a mechanical unit. Mechanical/automated cleaning methods remove soiling and microorganisms through an automated cleaning and rinsing process, this includes ultrasonic cleaning and washing. In the field of detergency, usually the term “stains” is used with reference to laundry, e.g., cleaning for textiles, fabric, or fibers, whereas the term “soils” is usually used with reference to hard surface cleaning, e.g., cleaning of dishes and cutlery. However, herein the terms “stain” and “soil” shall be used interchangeably.

A “sequestering builder” as used herein is different from a precipitating builder in that no significant amount of precipitate is formed when the builder is used in an amount sufficient to combine with all of the calcium ions in an aqueous solution with 7°dH hardness (German hardness) initially at neutral pH. A “strong builder” is classified as high efficiency chelators that can bind the divalent cations such as Ca2+strongly with a logarithmic stability constant (Log Kca) of the cat-ion/chelator complex of above 4, particular above 5, above 6 or above 7. The stability constants are determined at an ionic strength of 0.1 M and at a temperature of 25° C. A “strong sequestering builder” combines both of the above-mentioned properties.

### DETAILED DESCRIPTION

In the present invention new amylase enzymes are provided. More specifically, amylase variants, methods of making the amylase variants, compositions comprising the amylase variants, and methods of using the amylase variants or compositions comprising the amylase variants are provided.

### Amylase Variant

The present invention is directed to an amylase variant of a parent amylase, wherein said variant comprises
(i) an amino acid substitution at position 4 according to the numbering of SEQ ID NO: 3,
(ii) an amino acid substitution at one or more, preferably two or more, amino acid positions corresponding to amino acid positions selected from the group consisting of 25, 176, 186, 251, 405, and 482 according to the numbering of SEQ ID NO: 3, and
(iii) at least 60%, preferably at least 91% identity, but less than 100% sequence identity with SEQ ID NO: 1, 3, 4, or in any of SEQ ID NO: 15-41, preferably of SEQ ID NO: 1 or SEQ ID NO: 3, preferably SEQ ID NO: 1, preferably wherein the parent amylase of the amylase variant is an amylase according to SEQ ID NO: 1 or SEQ ID NO: 3, preferably SEQ ID NO: 1.

The amylase variant of the present invention is a non-naturally occurring amylase. Preferably, the amylase variant is an alpha-amylase. Preferably, the amylase variant of the present invention is a purified, isolated, synthetic, and/or recombinant amylase variant. Preferably, the amylase variant of the present invention is a purified and recombinant amylase variant.

Amylases and amylase variants according to the invention have “amylolytic activity” or “amylase activity”. “Amylolytic activity” or “amylase activity” describes the capability for the hydrolysis of glucosidic linkages in polysaccharides. Amylase activity may be determined by assays for measurement of amylase activity which are known to those skilled in the art. Examples for assays measuring amylase activity are the Phadebas assay or the EPS assay (“Infinity reagent”). In the Phadebas assay amylase activity is determined by employing Phadebas tablets as substrate (Phadebas Amylase Test, supplied by Magle Life Science). Starch is hydrolyzed by the amylase giving soluble blue fragments. The absorbance of the resulting blue solution, measured spectrophotometrically at 620 nm, is a function of the amylase activity. The measured absorbance is directly proportional to the specific activity (activity/mg of pure amylase protein) of the amylase in question under the given set of conditions.

Alternatively, amylase activity can also be determined by a method employing the Ethyliden-4-nitrophenyl-alpha-D-maltoheptaosid (EPS). D-maltoheptaoside is a blocked oligosaccharide which can be cleaved by an endo-amylase. Following the cleavage, the alpha-glucosidase included in the kit to digest the substrate to liberate a free PNP molecule which has a yellow color and thus can be measured by visible spectophotometry at 405 nm. Kits containing EPS substrate and alpha-glucosidase is manufactured for example by Roche Costum Biotech (cat. No. 10880078103). The slope of the time dependent absorption-curve is directly proportional to the specific activity (activity per mg enzyme) of the amylase in question under the given set of conditions.

In one embodiment, the amylase variant of the present invention exhibits at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 120%, at least 130%, at least 140%, at least 150%, at least 160%, at least 170%, at least 180%, at least 190%, or at least 200% of the amylolytic activity of the parent amylase. In one embodiment, the amylase variant of the present invention exhibits the same or an increased amylolytic activity compared to the parent amylase. Preferably, the amylase variant of the present invention exhibits an increased amylolytic activity compared to the parent amylase

Preferably, the parent amylase for the amylase variant of the present invention is an amylase having at least 60% sequence identity to any of SEQ ID NO: 1, 3, 4, or any of SEQ ID NO: 15-41, preferably the parent amylase for the amylase variant of the present invention is an amylase according to any of SEQ ID NO: 1, 3, 4, or any of SEQ ID NO: 15-41. Most preferably the parent amylase for the amylase variant is an amylase according to SEQ ID NO: 1 or SEQ ID NO: 3, preferably SEQ ID NO: 1.

The present invention is directed to an amylase variant comprising an amino acid alteration, preferably insertion, deletion, substitution, or combination thereof, most preferably substitution, at two or more positions corresponding to positions selected from the group consisting of 4, 25, 176, 186, 251, 405, and 482 according to the numbering of the amino acid sequence set forth in SEQ ID NO: 3.

In an alternative way, one can describe the amino acid positions 4, 25, 176, 186, 251, 405, and 482 (according to the numbering of SEQ ID NO: 3) with reference to the numbering of SEQ ID NO: 1, i.e., according to the numbering of SEQ ID NO: 1. In this alternative way of describing the amino acid positions of the present invention, amino acid positions 4, 25, 176, 186, 251, 405, and 482 according to the numbering of SEQ ID NO: 3 correspond to amino acid positions 4, 25, 176, 184, 249, 403, and 480 according to the numbering of SEQ ID NO: 1.

Preferably, the parent amylase for the amylase variant of the present invention is an amylase having at least 60% sequence identity to SEQ IDNO: 1, SEQ ID NO: 3, SEQ ID NO: 4, or any of SEQ ID NO: 15-41, preferably the parent amylase for the amylase variant of the present invention is an amylase according to SEQ IDNO: 1, SEQ ID NO: 3, SEQ ID NO: 4, or any of SEQ ID NO: 15-41. Most preferably the parent amylase for the amylase variant is an amylase according to SEQ ID NO: 1.

Preferably, the present invention is directed to an amylase variant comprising two or more amino acid substitutions selected from the group consisting of X4Q, X25H, X176K, X186E, X251E, X405M, and X482W according to the numbering of SEQ ID NO: 3.

Preferably, the present invention is directed to an amylase variant comprising compared to a parent sequence two or more amino acid substitutions selected from the group consisting of X4Q, X25H, X176K, X186E, X251E, X405M, X482W, and amino acid substitutions at these positions towards amino acids similar to the indicated amino acid substitutions, i.e., amino acids similar to the indicated target amino acids, at these positions according to the numbering of SEQ ID NO: 3 and wherein said variant has amylase activity, preferably wherein the parent amylase for the amylase variant of the present invention is an amylase according to SEQ ID NO: 1 or any amylase having at least 60% sequence identity to SEQ IDNO: 1, most preferably the parent amylase for the amylase variant is an amylase according to SEQ ID NO: 1, wherein
Amino acid A is similar to amino acids S
Amino acid D is similar to amino acids E; N
Amino acid E is similar to amino acids D; K; Q
Amino acid F is similar to amino acids W; Y
Amino acid H is similar to amino acids N; Y
Amino acid I is similar to amino acids L; M; V
Amino acid K is similar to amino acids E; Q; R
Amino acid L is similar to amino acids I; M; V
Amino acid M is similar to amino acids I; L; V
Amino acid N is similar to amino acids D; H; S
Amino acid Q is similar to amino acids E; K; R
Amino acid R is similar to amino acids K; Q
Amino acid S is similar to amino acids A; N; T
Amino acid T is similar to amino acids S
Amino acid V is similar to amino acids I; L; M
Amino acid W is similar to amino acids F; Y
Amino acid Y is similar to amino acids F; H; W.

Preferably, the present invention is directed to an amylase variant comprising compared to a parent sequence two or more amino acid substitutions selected from the group consisting of X4Q, X25H, X176K, X186E, X251E, X405M, and X482W according to the numbering of SEQ ID NO: 3 and wherein said variant has amylase activity, preferably wherein the parent amylase for the amylase variant of the present invention is an amylase according to SEQ ID NO: 1 or any amylase having at least 60% sequence identity to SEQ IDNO: 1, most preferably the parent amylase for the amylase variant is an amylase according to SEQ ID NO: 1.

Preferably, in this embodiment, the amino acid residue in the parent amylase at the above cited positions (i.e., X) corresponds to the amino acid residue shown in SEQ ID NO: 1 at the respective position according to the numbering of SEQ ID NO: 3.

Thus, preferably, the present invention is directed to an amylase variant comprising amino acid substitution at two or more positions corresponding to positions selected from the group consisting of G4, N25, R176, G186, T251, L405, and Y482 according to the numbering of SEQ ID NO: 3.

In a preferred embodiment, the present invention is directed to an amylase variant comprising an amino acid substitution at two or more positions corresponding to positions selected from the group consisting of G4Q, N25H, R176K, G186E, T251E, L405M, and Y482W according to the numbering of SEQ ID NO: 3.

The present invention is preferably directed to an amylase variant comprising an amino acid substitution at position 4 and at one or more positions corresponding to positions selected from the group consisting of 25, 176, 186, 251, 405, and 482 according to the numbering of SEQ ID NO: 3.

The present invention is preferably directed to an amylase variant comprising an amino acid substitution at position 4 and at two or more, three or more, four or more, five or more, or all positions corresponding to positions selected from the group consisting of 25, 176, 186, 251, 405, and 482 according to the numbering of SEQ ID NO: 3.

Preferably, the present invention is directed to an amylase variant comprising the amino acid substitution X4Q and one or more amino acid substitutions selected from the group consisting of

X25H, X176K, X186E, X251E, X405M, and X482W according to the numbering of SEQ ID NO: 3.

Preferably, the present invention is directed to an amylase variant comprising compared to a parent sequence the amino acid substitution X4Q and one or more amino acid substitutions selected from the group consisting of X25H, X176K, X186E, X251E, X405M, and X482W according to the numbering of SEQ ID NO: 3 and wherein said variant has amylase activity, preferably wherein the parent amylase for the amylase variant of the present invention is an amylase according to SEQ ID NO: 1 or any amylase having at least 60% sequence identity to SEQ IDNO: 1, most preferably the parent amylase for the amylase variant is an amylase according to SEQ ID NO: 1.

Preferably, in this embodiment, the amino acid residue in the parent amylase at the above cited positions (i.e., X) corresponds to the amino acid residue shown in SEQ ID NO: 1 at the respective position according to the numbering of SEQ ID NO: 3.

Thus, preferably, the present invention is directed to an amylase variant comprising an amino acid substitution at the amino acid position G4 and at one or more positions corresponding to positions selected from the group consisting of N25, R176, G186, T251, L405, and Y482 according to the numbering of SEQ ID NO: 3.

In a preferred embodiment, the present invention is directed to an amylase variant comprising the amino acid substitution G4Q and amino acid substitution at one or more positions corresponding to positions selected from the group consisting of N25H, R176K, G186E, T251E, L405M, and Y482W according to the numbering of SEQ ID NO: 3.

The present invention is preferably directed to an amylase variant comprising an amino acid substitution at position 4 and 25 and at one or more positions corresponding to positions selected from the group consisting of 176, 186, 251, 405, and 482 according to the numbering of SEQ ID NO: 3.

Preferably, the present invention is directed to an amylase variant comprising the amino acid substitution X4Q and X25H and one or more amino acid substitutions selected from the group consisting of X176K, X186E, X251E, X405M, and X482W according to the numbering of SEQ ID NO: 3.

Thus, preferably, the present invention is directed to an amylase variant comprising an amino acid substitution at the amino acid position G4 and N25 and at one or more positions corresponding to positions selected from the group consisting of R176, G186, T251, L405, and Y482 according to the numbering of SEQ ID NO: 3.

In a preferred embodiment, the present invention is directed to an amylase variant comprising the amino acid substitution G4Q and N25H and amino acid substitution at one or more positions corresponding to positions selected from the group consisting of R176K, G186E, T251E, L405M, and Y482W according to the numbering of SEQ ID NO: 3.

In one embodiment, the amylase variant further comprises an amino acid substitution at position 439, preferably X439K, more preferably W439K (according to the numbering of SEQ ID NO: 3).

Preferably, the amylase variant comprises compared to the parent amylase an amino acid substitution at one or more of the amino acid positions (according to the numbering of SEQ ID NO: 3) described below. Preferably, the parent amylase for the amylase variant is an amylase according to any of SEQ ID NO: 1, 3, 4, or any of SEQ ID NO: 15-41, most preferably the parent amylase for the amylase variant is an amylase according to SEQ ID NO: 1, 3, or 4, preferably SEQ ID NO: 1. Preferably, the amino acid residue of the parent amylase at the cited positions (i.e., X) corresponds to the amino acid residue shown in SEQ ID NO: 1 at the respective position (according to the numbering of SEQ ID NO: 3).

The amylase variant comprises an amino acid substitution at position 4 (according to the numbering of SEQ ID NO: 3), preferably the substitution X4Q.

Preferably, the amylase variant comprises an amino acid substitution at position 25 (according to the numbering of SEQ ID NO: 3), preferably the substitution X25H. Alternatively, the acid substitution at position 25 is X25Y or X25D.

Preferably, the amylase variant comprises an amino acid substitution at position 176 (according to the numbering of SEQ ID NO: 3), preferably the substitution X176K.

Preferably, the amylase variant comprises an amino acid substitution at position 186 (according to the numbering of SEQ ID NO: 3), preferably the substitution X186E.

Preferably, the amylase variant comprises an amino acid substitution at position 251 (according to the numbering of SEQ ID NO: 3), preferably the substitution X251E.

Preferably, the amylase variant comprises an amino acid substitution at position 405 (according to the numbering of SEQ ID NO: 3), preferably the substitution X405M.

Preferably, the amylase variant comprises an amino acid substitution at position 482 (according to the numbering of SEQ ID NO: 3), preferably the substitution X482W.

The amylase variant of the present invention comprises
(i) an amino acid substitution at position 4 according to the numbering of SEQ ID NO: 3, and
(ii) an amino acid substitution at one or more, two or more, three or more, four or more, five or more, or all of the amino acid positions corresponding to amino acid positions selected from the group consisting of 25, 176, 186, 251, 405, and 482 according to the numbering of SEQ ID NO: 3.

In one preferred embodiment, the amylase variant of the present invention comprises
(i) an amino acid substitution at position 4 according to the numbering of SEQ ID NO: 3, and
(ii) an amino acid substitution at one or more, two or more, or all of the amino acid positions corresponding to amino acid positions selected from the group consisting of 25, 176, and 186 according to the numbering of SEQ ID NO: 3.

In one preferred embodiment, the amylase variant of the present invention comprises
(i) an amino acid substitution at position 4 according to the numbering of SEQ ID NO: 3, and
(ii) an amino acid substitution at one or more, two or more, or all of the amino acid positions corresponding to amino acid positions selected from the group consisting of 176 and 186 according to the numbering of SEQ ID NO: 3.

In one preferred embodiment, the amylase variant of the present invention comprises
(i) an amino acid substitution at position 4 according to the numbering of SEQ ID NO: 3, and
(ii) an amino acid substitution at one or all of the amino acid positions corresponding to amino acid positions selected from the group consisting of 251 and 405 according to the numbering of SEQ ID NO: 3.

In one preferred embodiment, the amylase variant of the present invention comprises
(i) an amino acid substitution at position 4 according to the numbering of SEQ ID NO: 3,
(ii) an amino acid substitution at one or more, two or more, or all of the amino acid positions corresponding to amino acid positions selected from the group consisting of 25, 176, and 186 according to the numbering of SEQ ID NO: 3, and
(iii) an amino acid substitution at one or all of the amino acid positions corresponding to amino acid positions selected from the group consisting of 251 and 405 according to the numbering of SEQ ID NO: 3.

In a particularly preferred embodiment, the amylase variant of the present invention comprises
(i) an amino acid substitution at position 4 according to the numbering of SEQ ID NO: 3,
(ii) an amino acid substitution at one or more, two or more, or all of the amino acid positions corresponding to amino acid positions selected from the group consisting of 25, 176, and 186 according to the numbering of SEQ ID NO: 3,
(iii) an amino acid substitution at one or all of the amino acid positions corresponding to amino acid positions selected from the group consisting of 251 and 405 according to the numbering of SEQ ID NO: 3, and
(iv) an amino acid substitution at amino acid position corresponding to amino acid position 482 according to the numbering of SEQ ID NO: 3.

Preferably, the amylase variant of the present invention comprises amino acid substitutions at one of the following combinations of amino acid positions (according to the numbering of SEQ ID NO: 3):

4 + 25
4 + 176
4 + 186
4 + 251
4 + 405
4 + 482
4 + 25 + 176
4 + 25 + 186
4 + 25 + 251
4 + 25 + 405
4 + 25 + 482
4 + 176 + 186
4 + 176 + 251
4 + 176 + 405
4 + 176 + 482
4 + 186 + 251
4 + 186 + 405
4 + 186 + 482
4 + 251 + 405
4 + 251 + 482
4 + 405 + 482
4 + 25 + 176 + 186
4 + 25 + 176 + 251
4 + 25 + 176 + 405
4 + 25 + 176 + 482
4 + 25 + 186 + 251
4 + 25 + 186 + 405
4 + 25 + 186 + 482
4 + 25 + 251 + 405
4 + 25 + 251 + 482
4 + 25 + 405 + 482
4 + 176 + 186 + 251
4 + 176 + 186 + 405
4 + 176 + 186 + 482
4 + 176 + 251 + 405
4 + 176 + 251 + 482
4 + 176 + 405 + 482
4 + 186 + 251 + 405
4 + 186 + 251 + 482
4 + 186 + 405 + 482
4 + 251 + 405 + 482
4 + 25 + 176 + 186 + 251
4 + 25 + 176 + 186 + 405
4 + 25 + 176 + 186 + 482
4 + 25 + 176 + 251 + 405
4 + 25 + 176 + 251 + 482
4 + 25 + 176 + 405 + 482
4 + 25 + 186 + 251 + 405
4 + 25 + 186 + 251 + 482
4 + 25 + 186 + 405 + 482
4 + 25 + 251 + 405 + 482
4 + 176 + 186 + 251 + 405
4 + 176 + 186 + 251 + 482
4 + 176 + 186 + 405 + 482
4 + 176 + 251 + 405 + 482
4 + 186 + 251 + 405 + 482
4 + 25 + 176 + 186 + 251 + 405
4 + 25 + 176 + 186 + 251 + 482
4 + 25 + 176 + 186 + 405 + 482
4 + 25 + 176 + 251 + 405 + 482
4 + 25 + 186 + 251 + 405 + 482
4 + 176 + 186 + 251 + 405 + 482
4 + 25 + 176 + 186 + 251 + 405 + 482

From the above table, combinations of amino acid substitutions at amino acid positions containing at least the amino acid position 25 are particularly preferred.

From the above table, combinations of amino acid substitutions at amino acid positions containing at least one, preferably two, more preferably all of the amino acid positions selected from the group consisting of 176 and 186 are even more preferred.

From the above table, combinations of amino acid substitutions at amino acid positions containing at least one, preferably two, more preferably all of the amino acid positions selected from the group consisting of 25, 176, and 186 are even more preferred.

The amylase variant of the present invention comprises
(i) an amino acid substitution at position X4G according to the numbering of SEQ ID NO: 3, and
(ii) an amino acid substitution at one or more, two or more, three or more, four or more, five or more, or all of the amino acid positions corresponding to amino acid positions selected from the group consisting of X25H, X176K, X186E, X251E, X405M, and X482W according to the numbering of SEQ ID NO: 3.

In one preferred embodiment, the amylase variant of the present invention comprises
(i) an amino acid substitution at position X4G according to the numbering of SEQ ID NO: 3,
(ii) an amino acid substitution at one or more, two or more, or all of the amino acid positions corresponding to amino acid positions selected from the group consisting of X25H, X176K, and X186E according to the numbering of SEQ ID NO: 3, and
(iii) an amino acid substitution at one or all of the amino acid positions corresponding to amino acid positions selected from the group consisting of X251E and X405M according to the numbering of SEQ ID NO: 3.

In a particularly preferred embodiment, the amylase variant of the present invention comprises
(i) an amino acid substitution at position X4G according to the numbering of SEQ ID NO: 3,
(ii) an amino acid substitution at one or more, two or more, or all of the amino acid positions corresponding to amino acid positions selected from the group consisting of X25H, X176K, and X186E according to the numbering of SEQ ID NO: 3,
(iii) an amino acid substitution at one or all of the amino acid positions corresponding to amino acid positions selected from the group consisting of X251E and X405M according to the numbering of SEQ ID NO: 3, and
(iv) an amino acid substitution at amino acid position corresponding to amino acid position X482W according to the numbering of SEQ ID NO: 3

Preferably, the amylase variant of the present invention comprises one of the following combinations of amino acid substitutions (according to the numbering of SEQ ID NO: 3):

X4Q + X25H
X4Q + X176K
X4Q + X186E
X4Q + X251E
X4Q + X405M
X4Q + X482W
X4Q + X25H + X176K
X4Q + X25H + X186E
X4Q + X25H + X251E
X4Q + X25H + X405M
X4Q + X25H + X482W
X4Q + X176K + X186E
X4Q + X176K + X251E
X4Q + X176K + X405M
X4Q + X176K + X482W
X4Q + X186E + X251E
X4Q + X186E + X405M
X4Q + X186E + X482W
X4Q + X251E + X405M
X4Q + X251E + X482W
X4Q + X405M + X482W
X4Q + X25H + X176K + X186E
X4Q + X25H + X176K + X251E
X4Q + X25H + X176K + X405M
X4Q + X25H + X176K + X482W
X4Q + X25H + X186E + X251E
X4Q + X25H + X186E + X405M
X4Q + X25H + X186E + X482W
X4Q + X25H + X251E + X405M
X4Q + X25H + X251E + X482W
X4Q + X25H + X405M + X482W
X4Q + X176K + X186E + X251E
X4Q + X176K + X186E + X405M
X4Q + X176K + X186E + X482W
X4Q + X176K + X251E + X405M
X4Q + X176K + X251E + X482W
X4Q + X176K + X405M + X482W
X4Q + X186E + X251E + X405M
X4Q + X186E + X251E + X482W
X4Q + X186E + X405M + X482W
X4Q + X251E + X405M + X482W
X4Q + X25H + X176K + X186E + X251E
X4Q + X25H + X176K + X186E + X405M
X4Q + X25H + X176K + X186E + X482W
X4Q + X25H + X176K + X251E + X405M
X4Q + X25H + X176K + X251E + X482W
X4Q + X25H + X176K + X405M + X482W
X4Q + X25H + X186E + X251E + X405M
X4Q + X25H + X186E + X251E + X482W
X4Q + X25H + X186E + X405M + X482W
X4Q + X25H + X251E + X405M + X482W
X4Q + X176K + X186E + X251E + X405M
X4Q + X176K + X186E + X251E + X482W
X4Q + X176K + X186E + X405M + X482W
X4Q + X176K + X251E + X405M + X482W
X4Q + X186E + X251E + X405M + X482W
X4Q + X25H + X176K + X186E + X251E + X405M
X4Q + X25H + X176K + X186E + X251E + X482W
X4Q + X25H + X176K + X186E + X405M + X482W
X4Q + X25H + X176K + X251E + X405M + X482W
X4Q + X25H + X186E + X251E + X405M + X482W
X4Q + X176K + X186E + X251E + X405M + X482W
X4Q + X25H + X176K + X186E + X251E + X405M + X482W

From the above table, combinations of amino acid substitutions containing at least the amino acid substitution X25H are particularly preferred.

X4Q + X25H
X4Q + X25H + X176K
X4Q + X25H + X186E
X4Q + X25H + X251E
X4Q + X25H + X405M
X4Q + X25H + X482W
X4Q + X25H + X176K + X186E
X4Q + X25H + X176K + X251E
X4Q + X25H + X176K + X405M
X4Q + X25H + X176K + X482W
X4Q + X25H + X186E + X251E
X4Q + X25H + X186E + X405M
X4Q + X25H + X186E + X482W
X4Q + X25H + X251E + X405M
X4Q + X25H + X251E + X482W
X4Q + X25H + X405M + X482W
X4Q + X25H + X176K + X186E + X251E
X4Q + X25H + X176K + X186E + X405M
X4Q + X25H + X176K + X186E + X482W
X4Q + X25H + X176K + X251E + X405M
X4Q + X25H + X176K + X251E + X482W
X4Q + X25H + X176K + X405M + X482W
X4Q + X25H + X186E + X251E + X405M
X4Q + X25H + X186E + X251E + X482W
X4Q + X25H + X186E + X405M + X482W
X4Q + X25H + X251E + X405M + X482W
X4Q + X25H + X176K + X186E + X251E + X405M
X4Q + X25H + X176K + X186E + X251E + X482W
X4Q + X25H + X176K + X186E + X405M + X482W
X4Q + X25H + X176K + X251E + X405M + X482W
X4Q + X25H + X186E + X251E + X405M + X482W
X4Q + X25H + X176K + X186E + X251E + X405M + X482W

5 From the above table, combinations amino acid substitutions containing at least one, preferably two, more preferably all of the amino acid substitutions selected from the group consisting of X25H, X176K, and X186E are particularly preferred.

From the above table, combinations amino acid substitutions containing at least one, preferably two, more preferably all of the amino acid substitutions selected from the group consisting of X176K and X186E are particularly preferred.

Preferably, the amylase variant of the present invention comprises one of the following combinations of amino acid substitutions (according to the numbering of SEQ ID NO: 3):

X4Q + X176K + X186E
X4Q + X25H + X176K + X186E
X4Q + X25H + X176K + X186E + X251E
X4Q + X25H + X176K + X186E + X405M
X4Q + X25H + X176K + X186E + X482W
X4Q + X25H + X176K + X186E + X251E + X405M
X4Q + X25H + X176K + X186E + X251E + X482W
X4Q + X25H + X176K + X186E + X405M + X482W
X4Q + X25H + X176K + X186E + X251E + X405M + X482W

In one embodiment, the amylase variant further comprises an amino acid substitution at position 439, preferably X439K, more preferably W439K (according to the numbering of SEQ ID NO: 3), preferably the combination of amino acid substitutions (according to the numbering of SEQ ID NO: 3) X4Q+X25H+X176K+X186E+X251E+X405M+X439K+X482W.

Most preferred, the amylase variant of the present invention comprises the following combination of amino acid substitutions X4Q+X25H+X176K+X186E+X251E+X405M+X482W (according to the numbering of SEQ ID NO: 3).

Preferably, the amylase variant additionally comprises a deletion at one or more, preferably at two or more, amino acids corresponding to positions selected from the group consisting of 181, 182, 183 and 184. Preferably, the amylase variant of the present invention having one or more amino acid substitutions as described herein comprises a deletion of one or more amino acids corresponding to positions 183 and 184, preferably a deletion of both amino acids corresponding to positions 183 and 184 (according to the numbering of SEQ ID NO: 3). Preferably, the amylase variant of the present invention having one or more amino acid substitutions as described herein comprises a deletion of one or more, preferably of two or more, most preferably of two, amino acids corresponding to positions selected from the group consisting of R181, G182, D183, and G184, preferably D183* and G184*, wherein the numbering is according to the amino acid sequence set forth in SEQ ID NO: 3.

Preferably, the amylase variant comprises a deletion at two amino acids corresponding to positions selected from the group consisting of 181, 182, 183 and 184, preferably a deletion of amino acids corresponding to positions 181 and 182, 182 and 183, or 183 and 184, preferably the deletion D183* and G184*, wherein the numbering is according to SEQ ID NO: 3.

The amylase variant according to the present invention having one or more amino acid substitutions as described herein preferably has at least 65%, at least 70%, at least 75%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% sequence identity to the amino acid sequence of the parent amylase.

Preferably, the amylase variant according to the present invention having one or more of the amino acid substitutions described herein preferably has at least 65%, at least 70%, at least 75%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% sequence identity to the amino acid sequence set forth in SEQ ID NO: 1, 3, 4, or in any of SEQ ID NO 15-41, preferably, SEQ ID NO: 1, SEQ ID NO: 3, or SEQ ID NO: 4, more preferably SEQ ID NO: 1 or 3, most preferably SEQ ID NO: 1.

Preferably, the amylase variant according to the present invention having one or more of the amino acid substitutions described herein preferably has at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% sequence identity to the amino acid sequence set forth in SEQ ID NO: 1, 3, 4, or in any of SEQ ID NO 15-41, preferably, SEQ ID NO: 1, SEQ ID NO: 3, or SEQ ID NO: 4, more preferably SEQ ID NO: 1 or 3, most preferably SEQ ID NO: 1.

In one embodiment, the amylase variant according to the present invention having one or more of the amino acid substitutions described herein preferably has at least 65%, at least 70%, at least 75%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% sequence identity to the amino acid sequence set forth in SEQ ID NO: 1.

In another embodiment, the amylase variant according to the present invention having one or more of the amino acid substitutions described herein preferably has at least 65%, at least 70%, at least 75%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% sequence identity to the amino acid sequence set forth in SEQ ID NO: 3.

In another embodiment, the amylase variant according to the present invention having one or more of the amino acid substitutions described herein preferably has at least 65%, at least 70%, at least 75%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% sequence identity to the amino acid sequence set forth in SEQ ID NO: 4.

In another embodiment, the amylase variant according to the present invention having one or more of the amino acid substitutions described herein preferably has at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100% sequence identity to the amino acid sequence set forth in SEQ ID NO: 15.

In another embodiment, the amylase variant according to the present invention having one or more of the amino acid substitutions described herein preferably has at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100% sequence identity to the amino acid sequence set forth in SEQ ID NO: 16.

In another embodiment, the amylase variant according to the present invention having one or more of the amino acid substitutions described herein preferably has at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100% sequence identity to the amino acid sequence set forth in SEQ ID NO: 17.

In another embodiment, the amylase variant according to the present invention having one or more of the amino acid substitutions described herein preferably has at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100% sequence identity to the amino acid sequence set forth in SEQ ID NO: 18.

In another embodiment, the amylase variant according to the present invention having one or more of the amino acid substitutions described herein preferably has at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100% sequence identity to the amino acid sequence set forth in SEQ ID NO: 19.

In another embodiment, the amylase variant according to the present invention having one or more of the amino acid substitutions described herein preferably has at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100% sequence identity to the amino acid sequence set forth in SEQ ID NO: 20.

In another embodiment, the amylase variant according to the present invention having one or more of the amino acid substitutions described herein preferably has at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100% sequence identity to the amino acid sequence set forth in SEQ ID NO: 21.

In another embodiment, the amylase variant according to the present invention having one or more of the amino acid substitutions described herein preferably has at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100% sequence identity to the amino acid sequence set forth in SEQ ID NO: 22.

In another embodiment, the amylase variant according to the present invention having one or more of the amino acid substitutions described herein preferably has at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100% sequence identity to the amino acid sequence set forth in SEQ ID NO: 23.

In another embodiment, the amylase variant according to the present invention having one or more of the amino acid substitutions described herein preferably has at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100% sequence identity to the amino acid sequence set forth in SEQ ID NO: 24.

In another embodiment, the amylase variant according to the present invention having one or more of the amino acid substitutions described herein preferably has at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100% sequence identity to the amino acid sequence set forth in SEQ ID NO: 25.

In another embodiment, the amylase variant according to the present invention having one or more of the amino acid substitutions described herein preferably has at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100% sequence identity to the amino acid sequence set forth in SEQ ID NO: 26.

In another embodiment, the amylase variant according to the present invention having one or more of the amino acid substitutions described herein preferably has at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100% sequence identity to the amino acid sequence set forth in SEQ ID NO: 27.

In another embodiment, the amylase variant according to the present invention having one or more of the amino acid substitutions described herein preferably has at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100% sequence identity to the amino acid sequence set forth in SEQ ID NO: 28.

In another embodiment, the amylase variant according to the present invention having one or more of the amino acid substitutions described herein preferably has at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100% sequence identity to the amino acid sequence set forth in SEQ ID NO: 29.

In another embodiment, the amylase variant according to the present invention having one or more of the amino acid substitutions described herein preferably has at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100% sequence identity to the amino acid sequence set forth in SEQ ID NO: 30.

In another embodiment, the amylase variant according to the present invention having one or more of the amino acid substitutions described herein preferably has at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100% sequence identity to the amino acid sequence set forth in SEQ ID NO: 31.

In another embodiment, the amylase variant according to the present invention having one or more of the amino acid substitutions described herein preferably has at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100% sequence identity to the amino acid sequence set forth in SEQ ID NO: 32.

In another embodiment, the amylase variant according to the present invention having one or more of the amino acid substitutions described herein preferably has at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100% sequence identity to the amino acid sequence set forth in SEQ ID NO: 33.

In another embodiment, the amylase variant according to the present invention having one or more of the amino acid substitutions described herein preferably has at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100% sequence identity to the amino acid sequence set forth in SEQ ID NO: 34.

In another embodiment, the amylase variant according to the present invention having one or more of the amino acid substitutions described herein preferably has at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100% sequence identity to the amino acid sequence set forth in SEQ ID NO: 35.

In another embodiment, the amylase variant according to the present invention having one or more of the amino acid substitutions described herein preferably has at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100% sequence identity to the amino acid sequence set forth in SEQ ID NO: 36.

In another embodiment, the amylase variant according to the present invention having one or more of the amino acid substitutions described herein preferably has at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100% sequence identity to the amino acid sequence set forth in SEQ ID NO: 37.

In another embodiment, the amylase variant according to the present invention having one or more of the amino acid substitutions described herein preferably has at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100% sequence identity to the amino acid sequence set forth in SEQ ID NO: 38.

In another embodiment, the amylase variant according to the present invention having one or more of the amino acid substitutions described herein preferably has at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100% sequence identity to the amino acid sequence set forth in SEQ ID NO: 39.

In another embodiment, the amylase variant according to the present invention having one or more of the amino acid substitutions described herein preferably has at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100% sequence identity to the amino acid sequence set forth in SEQ ID NO: 40.

In another embodiment, the amylase variant according to the present invention having one or more of the amino acid substitutions described herein preferably has at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100% sequence identity to the amino acid sequence set forth in SEQ ID NO: 41.

Preferably, the amylase variant according to the present invention having one or more of the amino acid substitutions described herein preferably has at least 65%, at least 70%, at least 75%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% sequence identity to the amino acid sequence set forth in SEQ ID NO: 4.

Preferably, the amylase variant according to the present invention having one or more of the amino acid substitutions described herein preferably has at least 65%, at least 70%, at least 75%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% sequence identity to the amino acid sequence set forth in SEQ ID NO: 3.

More preferably, the amylase variant according to the present invention having one or more of the amino acid substitutions described herein preferably has at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100% sequence identity to the amino acid sequence set forth in SEQ ID NO: 1.

The amylase variant according to the present invention having amylase activity preferably has at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% sequence identity or sequence similarity, preferably sequence identity, to the amino acid sequence set forth in SEQ ID NO: 1. Preferably, the amylase variant of the present invention having amylase activity comprises:
(a) an amino acid sequence having at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% sequence identity to SEQ ID NO: 1;
(b) an amino acid sequence encoded by a polynucleotide having at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% sequence identity with SEQ ID NO: 2,
(c) an amino acid sequence encoded by a polynucleotide that hybridizes under high stringency conditions with the complement of
   (i) a coding sequence of SEQ ID NO: 1; or
   (ii) a polynucleotide shown in SEQ ID NO: 2;
(d) an amino acid sequence encoded by a polynucleotide that having at least 95%, but less than 100% sequence identity to SEQ ID NO: 2, wherein the polynucleotide further differs to SEQ ID NO: 2 merely by the degeneration of the genetic code, or
(e) a fragment of (a), (b), (c), or (d) having amylase activity.

Preferably, the amylase variant according to the present invention having amylase activity has at least 91.0%, at least 91.5%, at least 92.0%, at least 92.5%, at least 93.0%, at least 93.5%, 94.0%, at least 94.5%, at least 95.0%, at least 95.5%, at least 96.0%, at least 96.5%, at least 97.0%, at least 97.5%, at least 98.0%, at least 98.5%, at least 99.0%, at least 99.5%, at least 99.6%, at least 99.7%, at least 99.8%, or at least 99.9%, but less than 100% sequence identity or sequence similarity, preferably sequence identity, to the amino acid sequence set forth in SEQ ID NO: 1, SEQ ID NO: 3, or SEQ ID NO: 4. Particularly preferred, the amylase variant according to the present invention having amylase activity has at least 91.0%, at least 91.5%, at least 92.0%, at least 92.5%, at least 93.0%, at least 93.5%, 94.0%, at least 94.5%, at least 95.0%, at least 95.5%, at least 96.0%, at least 96.5%, at least 97.0%, at least 97.5%, at least 98.0%, at least 98.5%, at least 99.0%, at least 99.5%, at least 99.6%, at least 99.7%, at least 99.8%, or at least 99.9%, but less than 100% sequence identity to the amino acid sequence set forth in SEQ ID NO: 1. More preferred, The amylase variant according to the present invention having amylase activity preferably has at least 95.0% but less than 100% sequence identity to the amino acid sequence set forth in SEQ ID NO: 1.

The structure of alpha-amylases comprises three distinct domains A, B and C, see, e.g., Ma-chius et al., 1995, J. Mol. Biol. 246:545-559. The alpha-amylase variant described herein may further comprise one or more non-catalytic CBMs (carbohydrate-binding modules, also called carbohydrate binding domain or specifically for amylases starch binding domains). CBMs can improve the association of the enzyme with the substrate. CBMs are attached to the C-domain. Preferably, the amylase of the present invention does not comprise a carbohydrate binding domain. Preferably, the alpha-amylase variant of the present invention consists only of the three domains being A, B, and C domain.

As used herein, the “A and B domain” or “AB domain” of an alpha-amylase corresponds to the amino acids aligning with the amino acids 1-399 of SEQ ID NO: 3. As used herein, the “C domain” of an alpha-amylase corresponds to amino acids aligning with the amino acids 400-485 of SEQ ID NO: 3.

Preferably, the amylase variant comprising one or more of the amino acid alteration, preferably insertion, deletion, substitution, or combinations thereof, preferably substitution, as described above is a hybrid amylase comprising its domains, in particular its AB domain and its C domain from different parent amylases. The amylase variant may be produced by substituting the C domain or a portion thereof of an amylase with the C domain or a portion thereof of another amylase. Preferably, the A and B domain of the amylase variant described herein has at least 75% identity, such as at least 78%, at least 80%, at least 85%, at least 90%, at least 91%, at least 92%, at least 93%, at least 94%, at least 95%, at least 96%, at least 97%, at least 98%, or at least 99%, but less than 100%, identity to the A and B domain of the amylase of SEQ ID NO: 3. Preferably, the A and B domain of the amylase variant described herein has at least 75% identity, preferably at least 78%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100%, identity to SEQ ID NO: 6, meaning that the amino acid sequence that form the A and B domain has at least 75% identity, preferably at least 78%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100%, sequence identity to SEQ ID NO: 6.

Preferably, the C domain of the amylase variant described herein comprises a C domain having at least 75% identity, preferably at least 78%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100%, identity to the C domain of the amylase of SEQ ID NO: 5.

Preferably, the C domain of the amylase variant described herein has at least 75% identity, preferably at least 78%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100%, identity to SEQ ID NO: 8.

In one embodiment of the present invention, the amino acid sequence forming the A and B domain has at least 75% identity, preferably at least 78%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100%, identity to the amino acid sequence of SEQ ID NO: 6, and the amino acid sequence forming the C domain has at least 75% identity, preferably at least 78%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% identity to SEQ ID NO: 8.

In one embodiment of the present invention, the amino acid sequence forming the A and B domain has 100% identity to the amino acid sequence of SEQ ID NO: 6, and the amino acid sequence forming the C domain has at least 75% identity, preferably at least 78%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% identity to SEQ ID NO: 8.

In one embodiment of the present invention, the amino acid sequence forming the A and B domain has at least 75% identity, preferably at least 78%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100%, identity to the amino acid sequence of SEQ ID NO: 6, and the amino acid sequence forming the C domain has 100% identity to SEQ ID NO: 8.

In one embodiment, the present invention is directed to a method of making amylase variant comprising the step of making a hybrid from at least two different amylases, wherein the hybrid comprises an A and B domain and a C domain and wherein the amino acid sequence of the A and B domain is at least 75%, preferably at least 78%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% identical to the amino acid sequence of SEQ ID NO: 6 and the amino acid sequence of the C domain is at least 75%, preferably at least 78%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% identical to the amino acid sequence of SEQ ID NO: 8 and introducing into the hybrid one or more amino acid alterations as described herein.

The amylase variant according to the present invention having amylase activity preferably has at least 65%, at least 70%, at least 75%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% sequence identity to the amino acid sequence set forth in SEQ ID NO: 1 and comprises an A and B domain and a C domain wherein the amino acid sequence of the A and B domain is at least 75%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100%, identical to the amino acid sequence of SEQ ID NO: 6 and the amino acid sequence of the C domain is at least 75%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100%, identical to the amino acid sequence of SEQ ID NO: 8.

In one embodiment, the amylase variant according to the present invention having amylase activity comprises or consists of the amino acid sequence set forth in any of SEQ ID NO: 1, 3, 4, or in any of SEQ ID NO: 15-41, preferably SEQ ID NO: 1 or SEQ ID NO: 3, preferably SEQ ID NO: 1, with one or more of the herein cited amino acid alterations.

Preferably, the amylase variant according to the present invention having amylase activity comprises or consists of the amino acid sequence set forth in any of SEQ ID NO: 1, 3, 4, or in any of SEQ ID NO: 15-41, preferably SEQ ID NO: 1 or SEQ ID NO: 3, preferably SEQ ID NO: 1, with the amino acid substitution X4Q according to the numbering of SEQ ID NO: 3 and with 1 to 6, preferably 2 to 6 or 3 to 6, such as 1, 2, 3, 4, 5, or 6 of the amino acid substitutions selected from the group consisting of X25H, X176K, X186E, X251E, X405M, and X482W according to the numbering of SEQ ID NO: 3, preferably including a deletion at one or more, preferably at two or more, amino acids corresponding to positions selected from the group consisting of 181, 182, 183 and 184 according to the numbering of SEQ ID NO: 3, as described herein.

In another embodiment, the amylase variant according to the present invention having amylase activity comprises or consists of the amino acid sequence set forth in any of SEQ ID NO: 1, 3, 4, or in any of SEQ ID NO: 15-41, preferably SEQ ID NO: 1 or SEQ ID NO: 3, preferably SEQ ID NO: 1, with one or more of the herein cited amino acid alterations and further comprises 1 to 50, preferably 1 to 30, 1 to 25, 1 to 20, 1 to 15, 1 to 10, or 1 to 5, 2 to 30, 2 to 25, 2 to 20, 2 to 15 2 to 10, 2 to 8, or 2 to 5, preferably 3 to 30, 3 to 25, 3 to 20, 3 to 15 3 to 10, 3 to 8, or 3 to 5, preferably, 4 to 30, 4 to 25, 4 to 20, 4 to 15, 4 to 10, or 4 to 8 conservative amino acid exchanges, preferably including a deletion at one or more, preferably at two or more, amino acids corresponding to positions selected from the group consisting of 181, 182, 183 and 184 according to the numbering of SEQ ID NO: 3, as described herein.

Preferably, the amylase variant according to the present invention having amylase activity comprises or consists of the amino acid sequence set forth in any of SEQ ID NO: 1, 3, 4, or in any of SEQ ID NO: 15-41, preferably SEQ ID NO: 1 or SEQ ID NO: 3, preferably SEQ ID NO: 1, with the amino acid substitution X4Q according to the numbering of SEQ ID NO: 3 and with 1 to 6, preferably 2 to 6 or 3 to 6, such as 1, 2, 3, 4, 5, or 6 of the amino acid substitutions selected from the group consisting of X25H, X176K, X186E, X251E, X405M, and X482W according to the numbering of SEQ ID NO: 3, and further comprises 1 to 50, preferably 1 to 30, 1 to 25, 1 to 20, 1 to 15, 1 to 10, or 1 to 5, 2 to 30, 2 to 25, 2 to 20, 2 to 15 2 to 10, 2 to 8, or 2 to 5, preferably 3 to 30, 3 to 25, 3 to 20, 3 to 15 3 to 10, 3 to 8, or 3 to 5, preferably, 4 to 30, 4 to 25, 4 to 20, 4 to 15, 4 to 10, or 4 to 8 conservative amino acid exchanges preferably including a deletion at one or more, preferably at two or more, amino acids corresponding to positions selected from the group consisting of 181, 182, 183 and 184 according to the numbering of SEQ ID NO: 3, as described herein.

Preferably, the amylase variant according to the present invention having amylase activity comprises or consists of the amino acid sequence set forth in SEQ ID NO: 1 with the amino acid substitution X4Q according to the numbering of SEQ ID NO: 3 and with 1 to 6, preferably 2 to 6 or 3 to 6, such as 1, 2, 3, 4, 5, or 6 of the amino acid substitutions selected from the group consisting of X25H, X176K, X186E, X251E, X405M, and X482W according to the numbering of SEQ ID NO: 3, and further comprises 1 to 10, preferably 1 to 5 conservative amino acid exchanges.

Conservative amino acid substitutions may occur over the full length of the sequence of the amylase variant. In one embodiment, such mutations are not pertaining the functional domains of the amylase variant. In one embodiment, conservative mutations are not pertaining the catalytic centers of the amylase variant.

In one embodiment, the amylase variant of the present invention exhibits one or more improved property, preferably compared to the parent amylase, preferably compared to an amylase as shown in SEQ ID NO: 1, 3, 4, or in any of SEQ ID NO 15-41, preferably, SEQ ID NO: 1, SEQ ID NO: 3, or SEQ ID NO: 4, more preferably SEQ ID NO: 1 or 3, most preferably SEQ ID NO: 1. Preferably, the improved property is expressed as an Improvement Factor (IF) of >1.0. Preferably, for improved thermostability and improved wash performance the improvement is expressed as an Improvement Factor. Preferably, the Improvement Factor is equal or greater 1.1, equal or greater 1.2, equal or greater 1.3, equal or greater 1.4, equal or greater 1.5, equal or greater 1.6, equal or greater 1.7, equal or greater 1.8, equal or greater 1.9, or equal or greater 2.0. Preferably, the IF for wash performance is equal or greater 1.1, equal or greater 1.2, or equal or greater 1.3. Preferably, the IF for thermostability is equal or greater 1.1, equal or greater 1.2, equal or greater 1.3, equal or greater 1.5, or equal or greater 2.0.

Alternatively, the improvement of the amylase property is indicated as percentage of improvement compared to the parent amylase. Preferably, the amylase variants of the present invention exhibit at least 0.5%, at least 1%, at least 2%, at least 3%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 120%, at least 130%, at least 140%, at least 150%, at least 160%, at least 170%, at least 180%, at least 190%, or at least 200% improved property, preferably compared to the parent amylase, preferably compared to an amylase as shown in SEQ ID NO: 1 or SEQ ID NO: 3, preferably SEQ ID NO: 1.

Alternatively, in particular for stability improvement, the improvement of the amylase property is indicated as residual activity after stability challenge. Preferably, storage stability is indicated as residual activity after storage under the respective storage conditions, preferably, after storage in a detergent composition (preferably in a laundry or dishwash detergent, preferably laundry detergent). Preferably, the residual activity of the amylase variant is increased compared to the parent amylase. Preferably, the residual activity of the amylase variant is at least 0.5%, at least 1%, at least 2%, at least 3%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 120%, at least 130%, at least 140%, at least 150%, at least 160%, at least 170%, at least 180%, at least 190%, or at least 200% improved compared to the parent amylase, preferably compared to an amylase as shown in SEQ ID NO: 1 or SEQ ID NO: 3, preferably SEQ ID NO: 1 or compared to an amylase as shown in SEQ ID NO: 33. The residual activity compared to the parent amylase can also be converted into an improvement factor by forming the ratio of the residual activity of the variant vs. the residual activity of the parent amylase.

Preferably, the improved property is one or more property selected from the group consisting of an increase in expression, activity, stability, thermostability, specific activity, substrate specificity, pH-dependent activity, pH stability, oxidative stability, catalytic efficiency, catalytic rate, chemical stability, pH activity, stability under storage conditions, substrate binding, substrate cleavage, substrate stability, surface properties, thermal activity, Ca2+ dependency, performance in a detergent, performance in a laundry detergent, performance in an ADW detergent. Preferably, the improved activity is improved specific activity, substrate specificity, pH-dependent activity, catalytic efficiency, catalytic rate, pH activity, substrate binding, substrate cleavage, thermal activity, Ca2+ dependency, wash performance, wash performance of a laundry detergent, and/or wash performance of an ADW detergent, wash performance at low temperature (preferably below 40° C., more preferably below 30° C., even more preferably below 25° C.). Preferably, the improved stability is improved thermostability, thermostability in a detergent composition (preferably in laundry or dishwash detergent composition, preferably laundry detergent composition), pH stability, oxidative stability, chemical stability, stability under storage conditions, substrate stability, and/or thermal activity. Preferably, the improved property is improved expression, improved solubility, improved thermostability, improved thermostability in a detergent composition, improved stability under storage in a detergent composition, and/or an improved performance wash performance. Preferably, the improved property is improved thermostability, preferably, improved thermostability in a detergent composition, improved stability under storage in a detergent composition, and/or an improved wash performance. Preferably, the improved property is improved thermostability, improved thermostability in a detergent composition, improved stability under storage conditions, preferably improved stability under storage in a detergent composition, preferably improved stability under storage in a laundry detergent and/or an improved stability under storage in an ADW detergent, improved wash performance, preferably improved wash performance of a laundry detergent, improved wash performance of an ADW detergent, and/or wash performance at low temperature (preferably below 40° C., more preferably below 30° C., even more preferably below 25° C.). Preferably, the improved property is improved storage stability and/or improved wash performance. Most preferably, the improved property is improved storage stability, preferably improved stability in a detergent composition, preferably compared to an amylase as shown in SEQ ID NO: 1 or SEQ ID NO: 3, preferably SEQ ID NO: 1 or relative to an amylase as shown in SEQ ID NO: 33.

Preferably, the present invention is directed to an amylase variant of a parent amylase, wherein said variant comprises
(i) an amino acid substitution at position 4 according to the numbering of SEQ ID NO: 3,
(ii) an amino acid substitution at one or more, preferably two or more, amino acid positions corresponding to amino acid positions selected from the group consisting of 25, 176, 186, 251, 405, and 482 according to the numbering of SEQ ID NO: 3,
(iii) at least 60%, preferably at least 91% identity, but less than 100% sequence identity with SEQ ID NO: 1, 3, 4, or with any of SEQ ID NO: 15-41, preferably with SEQ ID NO: 1 or SEQ ID NO: 3, preferably SEQ ID NO: 1, preferably wherein the parent amylase of the amylase variant is an amylase according to SEQ ID NO: 1 or SEQ ID NO: 3, preferably SEQ ID NO: 1, and
(iv) a deletion of one or more amino acids corresponding to positions selected from the group consisting of 181, 182, 183 and 184 corresponding to the numbering of SEQ ID NO: 3, preferably a deletion of two or more amino acids corresponding to positions selected from the group consisting of 181, 182, 183 and 184, preferably a deletion of amino acids corresponding to positions 181 and 182, 182 and 183, or 183 and 184, preferably the deletion D183* and G184* (according to the numbering of SEQ ID NO: 3).

Preferably, the present invention is directed to an amylase variant of a parent amylase, wherein said variant comprises
(i) an amino acid substitution at position 4 according to the numbering of SEQ ID NO: 3,
(ii) an amino acid substitution at one or more, preferably two or more, more preferably three or more, amino acid positions corresponding to amino acid positions selected from the group consisting of 25, 176, 186, 251, 405, and 482 according to the numbering of SEQ ID NO: 3,
(iii) at least 80%, preferably at least 91% identity, but less than 100% sequence identity with SEQ ID NO: 1, 3, 4, or with any of SEQ ID NO: 15-41, preferably with SEQ ID NO: 1 or SEQ ID NO: 3, preferably SEQ ID NO: 1, preferably wherein the parent amylase of the amylase variant is an amylase according to SEQ ID NO: 1 or SEQ ID NO: 3, preferably SEQ ID NO: 1, and
(iv) a deletion of one or more amino acids corresponding to positions selected from the group consisting of 181, 182, 183 and 184 corresponding to the numbering of SEQ ID NO: 3, preferably a deletion of two or more amino acids corresponding to positions selected from the group consisting of 181, 182, 183 and 184, preferably a deletion of amino acids corresponding to positions 181 and 182, 182 and 183, or 183 and 184, preferably the deletion D183* and G184* (according to the numbering of SEQ ID NO: 3).

Preferably, the amylase variant of the present invention comprises
(i) the amino acid substitution X4G according to the numbering of SEQ ID NO: 3,
(ii) one or more, two or more, three or more, four or more, five or more, or all of the amino acid substitutions selected from the group consisting of X25H, X176K, X186E, X251E, X405M, and X482W according to the numbering of SEQ ID NO: 3,
(iii) at least 60%, at least 65%, at least 70%, at least 75%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% sequence identity with SEQ ID NO: 1, 3, 4, or with any of SEQ ID NO: 15-41, preferably with SEQ ID NO: 1 or SEQ ID NO: 3, preferably SEQ ID NO: 1, and
(iv) a deletion of one or more amino acids corresponding to positions selected from the group consisting of 181, 182, 183 and 184 corresponding to the numbering of SEQ ID NO: 3, preferably a deletion of two or more amino acids corresponding to positions selected from the group consisting of 181, 182, 183 and 184, preferably a deletion of amino acids corresponding to positions 181 and 182, 182 and 183, or 183 and 184, preferably the deletion D183* and G184* (according to the numbering of SEQ ID NO: 3).

Preferably, the amylase variant of the present invention comprises
(i) the amino acid substitution X4G according to the numbering of SEQ ID NO: 3,
(ii) one or more, two or more, three or more, four or more, five or more, or all of the amino acid substitutions selected from the group consisting of X25H, X176K, X186E, X251E, X405M, and X482W according to the numbering of SEQ ID NO: 3,
(iii) at least 60%, at least 65%, at least 70%, at least 75%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% sequence identity with SEQ ID NO: 1 or SEQ ID NO: 3, preferably SEQ ID NO: 1, and
(iv) a deletion of one or more amino acids corresponding to positions selected from the group consisting of 181, 182, 183 and 184 corresponding to the numbering of SEQ ID NO: 3, preferably a deletion of two or more amino acids corresponding to positions selected from the group consisting of 181, 182, 183 and 184, preferably a deletion of amino acids corresponding to positions 181 and 182, 182 and 183, or 183 and 184, preferably the deletion D183* and G184* (according to the numbering of SEQ ID NO: 3).

Preferably, the amylase variant of the present invention comprises
(i) a combination of amino acid substitutions selected from the group consisting of (according to the numbering of SEQ ID NO: 3):

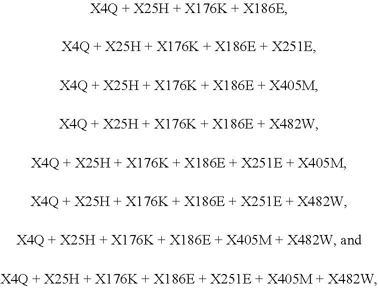

(ii) at least 60%, at least 65%, at least 70%, at least 75%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% sequence identity with SEQ ID NO: 1 or SEQ ID NO: 3, preferably SEQ ID NO: 1, and
(iii) a deletion of one or more amino acids corresponding to positions selected from the group consisting of 181, 182, 183 and 184 corresponding to the numbering of SEQ ID NO: 3, preferably a deletion of two or more amino acids corresponding to positions selected from the group consisting of 181, 182, 183 and 184, preferably a deletion of amino acids corresponding to positions 181 and 182, 182 and 183, or 183 and 184, preferably the deletion D183* and G184* (according to the numbering of SEQ ID NO: 3).

Preferably, the amylase variant of the present invention comprises
(i) the amino acid substitution X4G according to the numbering of SEQ ID NO: 3,
(ii) one or more, two or more, three or more, four or more, five or more, or all of the amino acid substitutions selected from the group consisting of X25H, X176K, X186E, X251E, X405M, and X482W according to the numbering of SEQ ID NO: 3,
(iii) at least 60%, at least 65%, at least 70%, at least 75%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% sequence identity with SEQ ID NO: 1 or SEQ ID NO: 3, preferably SEQ ID NO: 1,
(iv) a deletion of one or more amino acids corresponding to positions selected from the group consisting of 181, 182, 183 and 184 corresponding to the numbering of SEQ ID NO: 3, preferably a deletion of two or more amino acids corresponding to positions selected from the group consisting of 181, 182, 183 and 184, preferably a deletion of amino acids corresponding to positions 181 and 182, 182 and 183, or 183 and 184, preferably the deletion D183* and G184* (according to the numbering of SEQ ID NO: 3), and
(v) wherein the amylase variant exhibits one or more improved property, preferably relative to said parent amylase, preferably relative to the parent amylase set forth in SEQ ID NO: 1 or SEQ ID NO: 3, preferably SEQ ID NO: 1, or relative to an amylase as set forth in SEQ ID NO: 33, preferably the amylase variant has an increase in stability, thermostability, storage stability, storage stability in a detergent composition, wash performance, wash performance in a laundry detergent, and/or wash performance in a dish wash detergent, preferably, the improved property is improved storage stability and/or wash performance, preferably improved wash performance on laundry, preferably, the improved property is improved storage stability, preferably wherein said improved property is expressed as an Improvement Factor (IF) of >1.0 and wherein preferably the Improvement Factor is equal or greater than 1.1, preferably, equal or greater than 1.2, more preferably, equal or greater than 1.3, preferably the amylase variant exhibits improved storage stability in a detergent composition, preferably compared to an amylase with SEQ ID NO: 33, preferably with an improvement factor equal or greater than 1.1, preferably, equal or greater than 1.2, more preferably, equal or greater than 1.3, preferably after storage in a detergent composition as described herein at 37° C., preferably for 14 days, 28 days, 42 days, or 56 days, preferably for 56 days, or at 40° C., preferably for 14 days, 24 days, 56 days or 84 days, preferably 86 days, preferably as determined in ES1-C detergent as described herein, preferably containing an additional builder (preferably HEDP), preferably compared to a reference amylase, preferably compared to an amylase with an amino acid sequence shown in SEQ ID NO: 33; and/or preferably the amylase variant exhibits improved wash performance on laundry, preferably at 40° C., preferably compared to an amylase with an amino acid sequence shown in SEQ ID NO: 33.

Particularly preferred, the amylase variant of the present invention comprises
(i) a combination of amino acid substitutions selected from the group consisting of (according to the numbering of SEQ ID NO: 3):

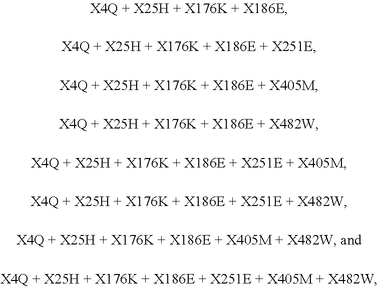

(ii) at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% sequence identity with SEQ ID NO: 1, and
(iii) a deletion of two amino acids corresponding to positions selected from the group consisting of 181, 182, 183 and 184, preferably a deletion of amino acids corresponding to positions 181 and 182, 182 and 183, or 183 and 184, preferably the deletion D183* and G184* (according to the numbering of SEQ ID NO: 3).

Especially preferred is an amylase variant comprising
(i) the combination of amino acid substitutions (according to the numbering of SEQ ID NO: 3):

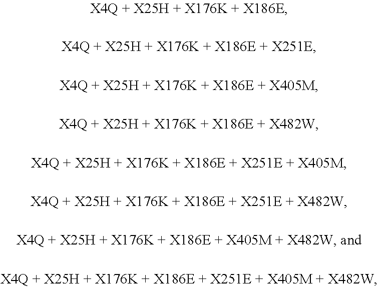

(ii) at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% sequence identity with SEQ ID NO: 1, and
(iii) a deletion of two amino acids corresponding to positions selected from the group consisting of 181, 182, 183 and 184, preferably a deletion of amino acids corresponding to positions 181 and 182, 182 and 183, or 183 and 184, preferably the deletion D183* and G184* (according to the numbering of SEQ ID NO: 3).

In one embodiment, the amylase variant further comprises an amino acid substitution at position 439, preferably X439K, more preferably W439K (according to the numbering of SEQ ID NO: 3), preferably the combination of amino acid substitutions (according to the numbering of SEQ ID NO: 3) X4Q+X25H+X176K+X186E+X251E+X405M+X439K+X482W.

Preferred is thus an amylase variant comprising or consisting of the amino acid sequence set forth in SEQ ID NO: 1 with the amino acid alterations X4Q+X25H+X176K+X186E+X251E+X405M+X439K+X482W according to the numbering of SEQ ID NO: 3.

Most preferred however is an amylase variant comprising or consisting of the amino acid sequence set forth in SEQ ID NO: 1 with the amino acid alterations X4Q+X25H+X176K+X186E+X251E+X405M+X482W according to the numbering of SEQ ID NO: 3.

Nucleic acid construct

The present invention also refers to a polynucleotide encoding the amylase variant of the present invention. Preferably, the polynucleotide is a codon-optimized polynucleotide for improving expression in a specific host cell, preferably a Bacillus cell.

The present invention thus also refers to a nucleic acid, preferably an isolated, a synthetic, and/or a recombinant nucleic acid comprising:
(a) a nucleic acid sequence having at least 60%, at least 65%, at least 70%, at least 75%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% identity to SEQ ID NO: 2, wherein the nucleic acid encodes an amylase variant having amylase activity described herein;
(b) a nucleic acid sequence encoding a polypeptide having at least 60%, at least 65%, at least 70%, at least 75%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% identity to SEQ ID NO: 1, wherein the nucleic acid encodes an amylase variant having amylase activity described herein;
(c) a polynucleotide that hybridizes under high stringency conditions, preferably under very high stringency conditions, with the complement of
   (i) a coding sequence of SEQ ID NO: 1 or a coding sequence of any amylase variant having amylase activity described herein; or
   (ii) a polynucleotide shown in SEQ ID NO: 2;
(d) a fragment of (a), (b), or (c), wherein the fragment encodes a polypeptide having amylase activity; or
(e) a nucleic acid sequence fully complementary to any of (a) to (d).
(f) a polynucleotide that differs from any of the nucleic acid sequences described in (a) to (e) merely by the degeneracy of the genetic code.

The present invention also refers to a nucleic acid construct, preferably an expression cassette, comprising the polynucleotide as described herein.

Typically, the expression cassette comprises three elements: a promoter sequence, an open reading frame, and a 3′ untranslated region that, in eukaryotes, usually contains a polyadenylation site. Additional regulatory elements may include transcriptional as well as translational enhancers. An intron sequence may also be added to the 5′ untranslated region (UTR) or in the coding sequence to increase the amount of the mature mRNA that accumulates in the cytosol. The expression cassette may be part of a vector or may be integrated into the genome of a host cell and replicated together with the genome of its host cell. The expression cassette usually is capable of increasing or decreasing expression.

The present invention also refers to an expression vector comprising the polynucleotide or the nucleic acid construct as described herein. The expression vector can be a low copy number vector or high copy number vector.

A vector as used herein may provide segments for transcription and translation of a foreign polynucleotide upon transformation into a host cell or host cell organelles. Such additional segments may include regulatory nucleotide sequences, one or more origins of replication that is required for its maintenance and/or replication in a specific cell type, one or more selectable markers, a polyadenylation signal, a suitable site for the insertion of foreign coding sequences such as a multiple cloning site etc. One example is when a vector is required to be maintained in a bacterial cell as an episomal genetic element (e.g., plasmid or cosmid molecule). Non-limiting examples of suitable origins of replication include the f1-ori and colE1.

A vector may replicate without integrating into the genome of a host cell, e.g., as a plasmid in a bacterial host cell, or it may integrate part or all of its DNA into the genome of the host cell and thus lead to replication and expression of its DNA.

The polynucleotide encoding the amylase variant may be introduced into a vector by means of standard recombinant DNA techniques. Once introduced into the vector, the polynucleotide comprising a coding sequence may be suitable to be introduced (transformed, transduced, transfected, etc.) into a host cell or host cell organelles. A cloning vector may be chosen suitable for expression of the polynucleotide sequence in the host cell or host cell organelles.

### Host Cell

The present invention also refers to a host cell comprising the polynucleotide encoding the amylase variant as described herein, the nucleic acid construct as described herein, or the expression vector as described herein. In one embodiment of the invention, a vector is used for transformation of a host cell.

The polynucleotide encoding the amylase variant as described herein may be transiently or stably introduced into a host cell and may be maintained non-integrated, for example, as a plasmid. Usually, stable transformation is due to integration of nucleic acid comprising a foreign coding sequence into the chromosomes or as an episome (separate piece of nuclear DNA). Usually, transient transformation is due to nucleic acid comprising a foreign nucleic acid sequence is not integrated into the chromosomes or as an episome. Alternatively, the polynucleotide encoding the amylase variant as described herein may be integrated into the host genome.

The introduction of nucleic acid into a host cell may, for instance, but not limited thereto, be ef-fected by protoplast transformation (see, e.g., Chang and Cohen, 1979, Molecular General Ge-netics 168:111-115), by using competent cells (see, e.g., Young and Spizizen, 1961, Journal of Bacteriology 81:823-829, or Dubnau and Davidoff-Abelson, 1971, Journal of Molecular Biology 56:209-221), by electroporation (see, e.g., Shigekawa and Dower, 1988, Biotechniques 6:742-751), or by conjugation (see, e.g., Koehler and Thorne, 1987, Journal of Bacteriology 169:5271-5278). Specific transformation protocols are known in the art for various types of host cells (see, e.g., for E. coli protoplast transformation see Hanahan, 1983, J. Mol. Biol. 166:557-580).

Various host cells can be used for expressing the nucleic acid construct described herein. Host cells comprising the genetic constructs described herein can be obtained by one of the methods described herein for introducing the polynucleotides into such host cells. The host cell of the present invention does not naturally express the amylase variant. Thus, the host cell is a recombinant host cell; the nucleic acid construct described herein is heterologous for the host cell. In one embodiment, the host cell is a prokaryote or a eukaryote. In another embodiment, the host cell is a bacteria, an archaea, a fungal cell, a yeast cell or a eukaryotic cell. In another embodiment, the host cell is a non-human host cell.

In one embodiment, the host cell is a bacterial cell. The bacterial host cell may be any grampositive bacterium or a gram-negative bacterium. Gram-positive bacteria include, but are not limited to, Bacillus, Brevibacterium, Corynebacterium, Streptococcus, Streptomyces, Staphylococcus, Enterococcus, Lactobacillus, Lactococcus, Clostridium, Geobacillus, and Oceanobacillus. Gram-negative bacteria include, but are not limited to, Escherichia, Pseudomonas, Salmonella, Campylobacter, Helicobacter, Acetobacter, Flavobacterium, Fusobacterium, Gluconobacter. In a specific embodiment, the bacterial host cell is a Echerichia coli cell. In one embodiment, the host cell is a bacterial cell. In a specific embodiment the host cell is of the genus Escherichia or Bacillus.

Preferably, the bacterial host cell is a Bacillus cell. The bacterial host cell may be any Bacillus cell. Bacillus cells useful in the practice of the present invention include, but are not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus methylotrophicus, Bacillus cereus Bacillus paralicheniformis, Bacillus subtilis, and Bacillus thuringiensis cells. In one embodiment, the bacterial host cell is a Bacillus amyloliquefaciens, Bacillus pumilus, Bacillus lentus, Bacillus licheniformis, Bacillus stearothermophilus or Bacillus subtilis cell. In preferred embodiment, the bacterial host cell is a Bacillus licheniformis cell, a Bacillus pumilus, or a Bacillus subtilis cell. Preferably, the bacterial host cell is a Bacillus licheniformis cell.

Alternatively, the bacterial host cell may be Lactobacillus acidophilus, Lactobacillus plantarum, Lactobacillus gasseri, Lactobacillus bulgaricusk, Lactobacillus reuteri, Escherichia coli, Staphylococcus aureus, Corynebacterium glutamicum, Corynebacterium acetoglutamicum, Corynebacterium acetoacidophilum, Corynebacterium callunae, Corynebacterium ammoniagenes, Corynebacterium thermoaminogenes, Corynebacterium melassecola, Corynebacterium effiziens, Corynebacterium efficiens, Corynebacterium deserti, Brevibacterium flavum, Brevibacterium lactofermentum, Brevibacterium divarecatum, Pseudomonas putida, Pseudomonas syringae, Streptomyces coelicolor, Streptomyces lividans, Streptomyces albus, Streptomyces avermitilis, Gluconobacter oxydans, Gluconobacter morbifer, Gluconobacter thailandicus, Acetobacter aceti, Clostridium acetobutylicum, Clostridium saccharobutylicum, Clostridium beijerinckii, Streptococcus equisimilis, Streptococcus pyogenes, Streptococcus uberis, Streptococcus equi subsp., Zooepidemicus or Basfia succiniciproducens.

Alternative further host cells include but are not limited to: Aspergillus niger, Aspergillus oryzae, Hansenula polymorpha, Thermomyces lanuginosus, Fusarium oxysporum, Fusarium heterosporum, Pichia pastoris (also known as Komagataella phaffii), Myceliopthora thermophile (C1), Themothelomyces thermophila, Schizosaccharomyces pombe, Trichoderma, preferably Trichoderma reesei and Saccharomyces, preferably Saccharomyces cerevisiae, or Rhizomucor. In another embodiment, the bacterial host cell may additionally contain modifications, e.g., deletions or disruptions, of other genes that may be detrimental to the production, recovery or application of a polypeptide of interest.

### Methods of Making

Another embodiment of the present invention is a method of obtaining an amylase variant of a parent amylase comprising the steps of:
a) introducing into a parent amylase, preferably into any of SEQ ID NO: 1, 3, 4, or in any of SEQ ID NO: 15-41, preferably into SEQ ID NO: 1 or SEQ ID NO: 3, preferably SEQ ID NO: 1,
(i) an amino acid substitution at position 4 according to the numbering of SEQ ID NO: 3, and
(ii) an amino acid substitution at one or more, preferably two or more, amino acid positions corresponding to amino acid positions selected from the group consisting of 25, 176, 186, 251, 405, and 482 according to the numbering of SEQ ID NO: 3, according to the numbering of SEQ ID NO: 3,
b) preferably introducing a deletion at two or more positions in the parent amylase selected from positions 181, 182, 183, and 184 according to the numbering of SEQ ID NO: 3;
and thereby providing an amylase variant of said parent amylase, wherein said variant has at least 60%, but less than 100% sequence identity to the amino acid sequence to the polypeptide of SEQ ID NO: 1, 3, 4, or to any of SEQ ID NO: 15-41, preferably to SEQ ID NO: 1, and wherein said amylase variant has amylase activity and preferably wherein the amylase variant has an improved property, preferably relative to said parent.

A preferred embodiment of the present invention is a method of obtaining an amylase variant of a parent amylase comprising the steps of:
a) introducing into a parent amylase, preferably into SEQ ID NO: 1 or SEQ ID NO: 3, preferably SEQ ID NO: 1,
(i) an amino acid substitution at position 4 according to the numbering of SEQ ID NO: 3, and
(ii) an amino acid substitution at one or more, preferably two or more, amino acid positions corresponding to amino acid positions selected from the group consisting of 25, 176, 186, 251, 405, and 482 according to the numbering of SEQ ID NO: 3,
b) preferably introducing a deletion at two or more positions in the parent amylase selected from positions 181, 182, 183, and 184 according to the numbering of SEQ ID NO: 3, and thereby providing an amylase variant of said parent amylase, wherein said variant has at least 80%, but less than 100% sequence identity to the amino acid sequence to the polypeptide of SEQ ID NO: 1, and wherein said amylase variant has amylase activity and preferably wherein the amylase variant has an improved property, preferably relative to said parent.

Ways of introducing amino acid alterations, e.g., a substitution or a deletion, into protein sequence are well known in the art. The variants may be prepared using any mutagenesis procedure known in the art, such as site-directed mutagenesis, synthetic gene construction, semi-synthetic gene construction, random mutagenesis, shuffling, etc.

The obtained amylase variant can be produced in an industrial scale and subsequently purified. Industrial production of enzymes usually is done by cultivating a host cell (also called fermentation) which expresses the enzyme. Suitable host cells are described herein. A nucleic acid sequence encoding the amylase variant described herein can be transformed into the host cell, which is subsequently cultivated under conditions suitable for the host cell to produce the amylase variant. In a preferred embodiment, the amylase variant is purified from the host cell.

Hence, in yet another embodiment, the present invention is directed to a method of producing an amylase variant, comprising the steps of
(a) providing a host cell comprising a heterologous nucleic acid construct comprising a polynucleotide encoding the amylase variant described herein by introducing the nucleic acid construct comprising the polynucleotide encoding the amylase variant as described herein into the host cell;
(b) cultivating the recombinant host cell of step (a) under conditions conductive for the expression of the polynucleotide; and
(c) optionally, recovering the amylase variant encoded by the polynucleotide.

Cultivation of the host cell normally takes place in a suitable nutrient medium allowing the recombinant cells to grow and express the desired protein. At the end of fermentation, the fermentation broth is collected and may be further processed, wherein the fermentation broth comprises a liquid fraction and a solid fraction. The enzyme of interest may be further purified from the fermentation broth.

The amylase variant described herein may be secreted (into the liquid fraction of the fermentation broth) or may not be secreted from the microbial cells (and therefore is comprised in the cells of the fermentation broth). Depending on this, the amylase variant may be recovered from the liquid fraction of the fermentation broth or from cell lysates. Preferably, the amylase variant is secreted from the cell into the fermentation broth, preferably by means of a secretion signal peptide added to the terminus of the amino acid sequence of the amylase variant. Recovery of the amylase variant can be achieved by methods known to those skilled in the art. Suitable methods for recovery of proteins from fermentation broth include but are not limited to collection, centrifugation, filtration, extraction, and precipitation. If the product of interest precipitates or crystallizes in the fermentation broth or binds at least in part to the particulate matter of the fermentation broth additional treatment steps might be needed to release the protein of interest from the biomass or to solubilize protein of interest crystals and precipitates. WO0043502A1, WO2008110498 A1, and WO2017097869A1 describe a method for recovering a protein of interest, which precipitates and/or crystallizes during fermentation, from the fermentation broth. In case the desired protein is comprised in the cells of the fermentation broth release of the product of interest from the cells might be needed. Release from the cells can be achieved for instance, but not being limited thereto, by cell lysis with techniques well known to the skilled person, e.g., lysozyme treatment, ultrasonic treatment, French press or combinations thereof. The amylase variant may be purified from the fermentation broth by methods known in the art. For example, the amylase variant may be isolated from the fermentation broth by conventional procedures including, but not limited to, centrifugation, filtration, extraction, spray-drying, evaporation, or precipitation. The isolated polypeptide may then be further purified by a variety of procedures known in the art including, but not limited to, chromatography (e.g., ion exchange, affinity, hydrophobic, chromatofocusing, and size exclusion), electrophoretic procedures (e.g., preparative isoelectric focusing (IEF), differential solubility (e.g., ammonium sulfate precipitation), or extraction (see, e.g., Protein Purification, J.-C. Janson and Lars Ryden, editors, VCH Publishers, New York, 1989). The purified polypeptide may then be concentrated by procedures known in the art including, but not limited to, ultrafiltration and evaporation, in particular, thin film evaporation.

### Amylase Preparation

The purified solution of the amylase variant described herein may be further processed to form an amylase containing composition. Hence, also claimed herein is a composition comprising the amylase variant described herein and at least one additional component.

Thus, the present invention therefore also refers to a method for making a composition comprisesing the steps of mixing
a) an amylase variant as described herein; and
b) one or more component described herein.

Further, the present invention therefore also refers to a method for improving amylase stability in a composition comprising the steps of mixing
a) an amylase variant as described herein; and
b) one or more component described herein.

The composition can be a non-complex formulation, e.g., an amylase variant formulation, or a complex formulation, e.g., a detergent composition.

In one embodiment of the present invention, the amylase variant is formulated as an amylase variant formulation, preferably a concentrated amylase variant formulation. The amylase variant formulation can be either solid or liquid. Protein formulations can be obtained by using techniques known in the art. For instance, without being limited thereto, solid enzyme formulations can be obtained by extrusion or granulation. Suitable extrusion and granulation techniques are known in the art and are described for instance in WO 94/19444 A1 and WO 97/43482 A1.

In one embodiment, the amylase variant formulation, in particular the liquid enzyme formulation, comprises in addition one or more additional compounds selected from the group consisting of solvent, salt, pH regulator, preservative, enzyme stabilizer, and thickening agent. Preferably, the amylase variant formulation is devoid of surfactants. The solvent may be water and/or an organic solvent. Aqueous amylase variant formulations of the invention may comprise water in amounts of more than about 50% by weight, more than about 60% by weight, more than about 70% by weight, or more than about 80% by weight, all relative to the total weight of the enzyme formulation. The amylase variant containing formulations of the invention may comprise an organic solvent in amounts of more than 30%, more than 40%, more than about 50% by weight, more than about 60% by weight, more than about 70% by weight, or more than about 80% by weight, all relative to the total weight of the enzyme formulation. The organic solvent may be a water-miscible solvent. The organic solvent may be one or more selected from the group consisting of glycerol, propanediol, polypropylene glycol, and polyethylene glycol.

In one embodiment, the amylase variant formulation comprises at least one preservative. Preferably, preservative means substances that are added to a liquid composition for the purpose of preservation, meaning more preferably that compounds known to have preserving features comprised in a liquid composition formed in the production process are excluded from the term preservatives. In one embodiment, the preservative is selected from the group consisting of 2-phenoxyethanol, glutaraldehyde, 2-bromo-2-nitropropane-1,3-diol, and formic acid in acid form or as its salt, and 4,4′-dichloro 2-hydroxydiphenylether. Usually, the liquid compositions of the invention comprise at least one preservative in amounts below 10 ppm, such as in amounts ranging from 2 ppm to 5% by weight relative to the total weight of the liquid composition. Alternatively, the amylase variant formulation is free from preservatives, meaning that preservatives are comprised in amounts less than 1 ppm, preferably 0 ppm.

Preferably, the amylase variant formulation comprises an enzyme stabilizing system. Preferably, the amylase variant formulation described herein comprises from about 0.001% to about 10%, from about 0.005% to about 8%, or from about 0.01% to about 6%, by weight of the composition, of an enzyme stabilizing system. The enzyme stabilizing system can be any stabilizing system which is compatible with the amylase.

Preferably, the enzyme stabilizing system comprises at least one compound selected from the group consisting of polyols (preferably, 1,3-propanediol, ethylene glycol, glycerol, 1,2-propanediol, or sorbitol), inorganic salts (preferably, CaCl2, MgCl2, or NaCl), short chain (preferably, C₁-C₃) carboxylic acids or salts thereof (preferably, formic acid, formate (preferably, sodium formate), acetic acid, acetate, or lactate), borate, boric acid, boronic acids (preferably, 4-formyl phenylboronic acid (4-FPBA)), peptide aldehydes (preferably, Z-VAL-H or Z-GAY-H), peptide acetals, and peptide aldehyde hydrosulfite adducts. Preferably, the enzyme stabilizing system comprises a combination of at least two of the compounds selected from the group consisting of salts, polyols, and short chain carboxylic acids and preferably one or more of the compounds selected from the group consisting of borate, boric acid, boronic acids (preferably, 4-formyl phenylboronic acid (4-FPBA)), peptide aldehydes, peptide acetals, and peptide aldehyde hydrosulfite adducts. In a preferred embodiment, the stabilizing system comprises a protease inhibitor in case a protease is present, preferably selected from borate, boric acid, boronic acids (preferably, 4-FPBA), peptide aldehydes (preferably, peptide aldehydes like Z-VAL-H or Z-GAY-H), peptide acetals, and peptide aldehyde hydrosulfite adducts, preferably the protease inhibitor is a peptide aldehyde, preferably Z-VAL-H or Z-GAY-H. In one embodiment, the stabilizing system does not comprise a protease inhibitor. Preferably, the composition is boron-free. Preferably, the amylase variant formulation comprises a calcium salt, preferably calcium chloride.

Preferably, the liquid amylase variant formulation comprises or consists of the amylase variant, a solvent, an enzyme stabilizing system, and optionally a preservative and optionally a second enzyme different from the amylase variant as described herein. Preferably, the amylase variant formulation is devoid of surfactants.

Thus, the present invention therefore also refers to a method for making an amylase variant formulation, preferably a concentrated amylase variant formulation, comprising the steps of mixing
a) an amylase variant as described herein; and
b) one or more component selected from the group consisting of solvent, enzyme stabilizing system, preservative, and a second enzyme different from the amylase variant.

Further, the present invention therefore also refers to a method for improving amylase stability in a formulation with comprising the steps of mixing
a) an amylase variant as described herein; and
b) one or more component selected from the group consisting of solvent, enzyme stabilizing system, preservative, and a second enzyme different from the amylase variant.

### Secondary Enzymes

In another embodiment, the composition comprising an amylase variant as described herein furthe comprises one or more second enzyme different from the amylase variant. Preferably, the second enzyme is selected from the group consisting of, proteases, second amylases, lipases, cellulases, mannanases, hemicellulases, phospholipases, esterases, pectinases, lactases, peroxidases, xylanases, cutinases, pectate lyases, keratinases, reductases, oxidases, phenoloxidases, lipoxygenases, ligninases, pullulanases, tannases, pentosanases, malanases, beta-glucanases, arabinosidases, hyaluronidases, chondroitinases, laccases, nucleases, DNase, phosphodiesterases, phytases, carbohydrases, galactanases, xanthanases, xyloglucanases, oxidoreductase, perhydrolases, aminopeptidase, asparaginase, carbohydrase, carboxypeptidase, catalase, chitinase, cyclodextrin glycosyltransferase, alpha-galactosidase, beta-galactosidase, glucoamylase, alpha-glucosidase, beta-glucosidase, invertase, ribonuclease, transglutaminase, and dispersins, and combinations of at least two of the foregoing types. More preferably, the second enzyme is selected from the group consisting of protease, lipases, cellulases, mannanases, xylanases, DNases, dispersins, pectinases, oxidoreductases, and cutinases, and combinations of at least two of the foregoing types. Most preferably, the second enzyme is protease, preferably, subtilisin protease.

The composition of the present invention can comprise one type of enzyme or more than one enzyme of different types, e.g., an amylase and a protease, or more than one enzyme of the same type, e.g., two or more different proteases, or mixtures thereof, e.g., an amylase and two different proteases.

### Proteases

Proteases are active proteins exerting “protease activity” or “proteolytic activity”. Proteolytic activity is related to the rate of degradation of protein by a protease or proteolytic enzyme in a defined course of time.

Preferably, the second enzyme different from the amylase variant is a protease with at least 40 to 100% identity to the full-length polypeptide sequence of any of SEQ ID NO: 10-14. In one embodiment, the protease comprises an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the full length polypeptide sequence shown in SEQ ID NO: 10, 11, 12,13, or 14, preferably SEQ ID NO: 10.

Preferably, the protease used in combination with the amylase variant described herein comprises an amino acid sequence having at least 80%, at least 85%, at least 90%, at least 91%, at least 92%, at least 93%, at least 94%, at least 95%, at least 96%, at least 97%, at least 98%, or at least 99%, but below 100% sequence identity with SEQ ID NO: 10 and further comprises amino acid substitutions in one or more of the following positions 3, 4, 9, 15, 24, 27, 33, 36, 57, 68, 76, 77, 87, 95, 96, 97, 98, 99, 100, 101, 102, 103, 104, 106, 118, 120, 123, 128, 129, 130, 131, 154, 160, 167, 170, 194, 199, 205, 206, 217, 218, 222, 224, 232, 235, 236, 245, 248, 252 and 274 (according to the BPN′ numbering) and which has proteolytic activity. In one embodiment, such a protease is not mutated at positions Asp32, His64 and Ser221 (according to BPN′ numbering). Preferably, the protease used in combination with the amylase variant described herein comprises an amino acid sequence having at least 80%, at least 85%, at least 90%, at least 91%, at least 92%, at least 93%, at least 94%, at least 95%, at least 96%, at least 97%, at least 98%, or at least 99%, but below 100% sequence identity with SEQ ID NO: 10 and is fur the characterized by having amino acid glutamic acid (E), or aspartic acid (D), or asparagine (N), or glutamine (Q), or alanine (A), or glycine (G), or serine(S), preferably glutamic acid (E), at position 101 (according to BPN′ numbering) and has proteolytic activity. Mostly preferred is a protease that has at least 80%, but below 100% sequence identity with SEQ ID NO: 10 and is characterized by having amino acid glutamic acid (E) at position 101 (according to BPN′ numbering) and that has proteolytic activity. The protease may comprise an amino acid substitution at position 101, such as R101E alone or in combination with one or more substitutions at positions 3, 4, 9, 15, 24, 27, 33, 36, 57, 68, 76, 77, 87, 95, 96, 97, 98, 99, 100, 102, 103, 104, 106, 118, 120, 123, 128, 129, 130, 131, 154, 160, 167, 170, 194, 199, 205, 206, 217, 218, 222, 224, 232, 235, 236, 245, 248, 252 and/or 274 (according to BPN′ numbering) and has proteolytic activity. In one embodiment, said protease comprises one or more further substitutions: (a) threonine at position 3 (3T), (b) isoleucine at position 4 (41), (c) alanine, threonine or arginine at position 63 (63A, 63T, or 63R), (d) aspartic acid or glutamic acid at position 156 (156D or 156E), (e) proline at position 194 (194P), (f) methionine at position 199 (199M), (g) isoleucine at position 205 (2051), (h) aspartic acid, glutamic acid or glycine at position 217 (217D, 217E or 217G), (i) combinations of two or more amino acids according to (a) to (h). A suitable protease may be at least 80% identical to SEQ ID NO: 10 and is characterized by comprising one amino acid (according to (a)-(h)) or combinations according to (i) together with the amino acid 101E, 101D, 101N, 101Q, 101A, 101G, or 101S (according to BPN′ numbering) and has proteolytic activity. In one embodiment, the protease is at least 80% identical to SEQ ID NO: 10 and is characterized by comprising the mutation (according to BPN′ numbering) R101E, or S3T+V41+V2051, or S3T+V41+R101E+V2051 or S3T+V41+V199M+V205| +L217D, and has proteolytic activity. In another embodiment, the protease comprises an amino acid sequence having at least 80% identity to SEQ ID NO: 10 and being further characterized by comprising S3T+V41+S9R+A15T+V68A+D99S+R101S+A103S+1104V+N218D (according to the BPN′ numbering) and has proteolytic activity. In another embodiment, the protease may have an amino acid sequence being at least 80% identical to SEQ ID NO:10 and being further characterized by comprising R101E, and one or more substitutions selected from the group consisting of S156D, L262E, Q137H, S3T, R45E,D,Q, P55N, T58W,Y,L, Q59D,M,N,T, G61 D,R, S87E, G97S, A98D,E,R, S106A, W, N117E, H120V,D,K,N, S125M, P129D, E136Q, S144W, S161T, S163A,G, Y171 L, A172S, N185Q, V199M, Y209W, M222Q, N238H, V244T, N261T, D and L262N,Q,D (according to the BPN′ numbering), and has proteolytic activity.

### Lipases

“Lipases”, “lipolytic enzyme”, “lipid esterase”, all refer to an enzyme of EC class 3.1.1 (“carboxylic ester hydrolase”). Lipase means active protein having lipase activity (or lipolytic activity; triacylglycerol lipase, EC 3.1.1.3), cutinase activity (EC 3.1.1.74; enzymes having cutinase activity may be called cutinase herein), sterol esterase activity (EC 3.1.1.13) and/or wax-ester hydrolase activity (EC 3.1.1.50). Lipases include those of bacterial or fungal origin.

In one aspect of the invention, a suitable lipase (component (b)) is selected from the following: lipases from Humicola (synonym Thermomyces), e.g. from H. lanuginosa (T. lanuginosus) as described in EP 258068, EP 305216, WO 92/05249 and WO 2009/109500 or from H. insolens as described in WO 96/13580; lipases derived from Rhizomucor miehei as described in WO 92/05249; lipase from strains of Pseudomonas (some of these now renamed to Burkhold-eria), e.g. from P. alcaligenes or P. pseudoalcaligenes (EP 218272, WO 94/25578, WO 95/30744, WO 95/35381, WO 96/00292), P. cepacia (EP 331376), P. stutzeri (GB 1372034), P. fluorescens, Pseudomonas sp. strain SD705 (WO 95/06720 and WO 96/27002), P. wiscon-sinensis (WO 96/12012), Pseudomonas mendocina (WO 95/14783), P. glumae (WO 95/35381, WO 96/00292); lipase from Streptomyces griseus (WO 2011/150157) and S. pristinaespi-ralis (WO 2012/137147), GDSL-type Streptomyces lipases (WO 2010/065455); lipase from Thermobifida fusca as disclosed in WO 2011/084412; lipase from Geobacillus stearothermophilus as disclosed in WO 2011/084417; Bacillus lipases, e.g. as disclosed in WO 00/60063, lipases from B. subtilis as disclosed in Dartois et al. (1992), Biochemica et Biophysica Acta, 1131, 253-360 or WO 2011/084599, B. stearothermophilus (JP S64-074992) or B. pumilus (WO 91/16422); lipase from Candida antarctica as disclosed in WO 94/01541; cutinase from Pseudomonas mendocina (U.S. Pat. No. 5,389,536, WO 88/09367); cutinase from Magnaporthe grisea (WO 2010/107560); cutinase from Fusarum solani pisi as disclosed in WO 90/09446, WO 00/34450 and WO 01/92502; and cutinase from Humicola lanuginosa as disclosed in WO 00/34450 and WO 01/92502.

Such suitable lipase variants are e.g. those which are developed by methods as disclosed in WO 95/22615, WO 97/04079, WO 97/07202, WO 00/60063, WO 2007/087508, EP 407225 and EP 260105.

Commercially available lipase enzymes include but are not limited to those sold under the trade names Lipolase™, Lipex™, Lipolex™ and Lipoclean™ (Novozymes A/S), Lumafast (origi-nally from Genencor), Preferenz L (DuPont), and Lipomax (Gist-Brocades/now DSM).

In one embodiment, lipase is selected from fungal triacylglycerol lipase (EC class 3.1.1.3). Fungal triacylglycerol lipase may be selected from lipases of Thermomyces lanuginosus. In one embodiment, the Thermomyces lanuginosa lipase is selected from triacylglycerol lipase according to amino acids 1-269 of SEQ ID NO: 2 of U.S. Pat. No. 5,869,438 and variants thereof having lipolytic activity.

Thermomyces lanuginosa lipase may be selected from variants having lipolytic activity which are at least 90%, at least 91%, at least 92%, at least 93%, at least 94%, at least 95%, at least 96%, at least 97%, at least 98%, at least 99%, or 100% identical when compared to the full length polypeptide sequence of amino acids 1-269 of SEQ ID NO: 2 of U.S. Pat. No. 5,869,438. Thermomyces lanuginosa lipase may be selected from variants having lipolytic activity comprisesing conservative mutations only, which do not pertain the functional domain of amino acids 1-269 of SEQ ID NO: 2 of U.S. Pat. No. 5,869,438. Lipase variants of this embodiment having lipolytic activity may be at least 95%, at least 96%, at least 97%, at least 98%, or at least 99% similar when compared to the full-length polypeptide sequence of amino acids 1-269 of SEQ ID NO: 2 of U.S. Pat. No. 5,869,438.

Thermomyces lanuginosa lipase may be selected from variants having lipolytic activity comprisesing the following amino acid substitutions when compared to amino acids 1-269 of SEQ ID NO: 2 of U.S. Pat. No. 5,869,438: T231R and N233R. Said lipase variants may further comprise one or more of the following amino acid exchanges when compared to amino acids 1-269 of SEQ ID NO: 2 of U.S. Pat. No. 5,869,438: Q4V, V60S, A150G, L227G, P256K.

Thermomyces lanuginosa lipase may be selected from variants having lipolytic activity comprisesing the amino acid substitutions T231R, N233R, Q4V, V60S, A150G, L227G, P256K within the polypeptide sequence of amino acids 1-269 of SEQ ID NO: 2 of U.S. Pat. No. 5,869,438 and are at least 95%, at least 96%, or at least 97% similar when compared to the full length polypeptide sequence of amino acids 1-269 of SEQ ID NO: 2 of U.S. Pat. No. 5,869,438.

Thermomyces lanuginosa lipase may be selected from variants having lipolytic activity comprisesing the amino acid substitutions T231R and N233R within amino acids 1-269 of SEQ ID NO: 2 of U.S. Pat. No. 5,869,438 and are at least 95%, at least 96%, at least 97%, at least 98%, or at least 99% similar when compared to the full length polypeptide sequence of amino acids 1-269 of SEQ ID NO: 2 of U.S. Pat. No. 5,869,438.

Thermomyces lanuginosa lipase may be a variant of amino acids 1-269 of SEQ ID NO: 2 of U.S. Pat. No. 5,869,438 having lipolytic activity, wherein the variant of amino acids 1-269 of SEQ ID NO: 2 of U.S. Pat. No. 5,869,438 is characterized in containing the amino acid substitutions T231R and N233R. Thermomyces lanuginosa lipase may be selected from variants having lipolytic activity preferably comprising at least one, preferably more than one, more preferably all of the following substitutions N11K, A18K, G23K, K24A, V771, D130A, V1541, V187T, T189Q within the polypeptide sequence of amino acids 1-269 of SEQ ID NO: 1 of WO2015/010009 and are at least 95%, at least 96%, or at least 97% similar when compared to the full length polypeptide sequence of amino acids 1-269 of SEQ ID NO: 1 of WO2015/010009.

### Amylases

Amylases different from the amylase described herein include those of bacterial or fungal origin (EC 3.2.1.1 and 3.2.1.2, respectively). Preferably, amylases are selected from the group of al-pha-amylases (EC 3.2.1.1).

Amylases maybe from Bacillus licheniformis having SEQ ID NO:2 as described in WO 95/10603 and variants at least 95% thereto. Suitable variants are described in WO 95/10603 comprising one or more substitutions in the following positions: 15, 23, 105, 106, 124, 128, 133, 154, 156, 178, 179, 181, 188, 190, 197, 201, 202, 207, 208, 209, 211, 243, 264, 304, 305, 391, 408, and 444 which have amylolytic activity. Variants are described in WO 94/02597, WO 94/018314, WO 97/043424 and SEQ ID NO:4 of WO 99/019467.

Amylases further maybe from B. stearothermophilus having SEQ ID NO:6 as disclosed in WO 02/10355 or an amylase with optionally having a C-terminal truncation over the wildtype sequence. Suitable variants of SEQ ID NO:6 include those comprising a deletion in positions 179 and/or 181 and/or 182 and/or a substitution in position 193.

Amylases further maybe from Bacillus sp. 707 having SEQ ID NO:6 as disclosed in WO 99/19467 and variants at least 95% thereto. Preferred variants of SEQ NO: 6 are those having a substitution, a deletion or an insertion in one or more of the following positions: R181, G182, H183, G184, N195, 1206, E212, E216 and K269.

Amylases further maybe from Bacillus halmapalus having SEQ ID NO:2 or SEQ ID NO:7 as described in WO 96/23872, also described herein as SP-722. Preferred variants are described in WO 97/3296, WO 99/194671 and WO 2013/001078.

Amylases further may be from Bacillus sp. DSM 12649 having SEQ ID NO:4 as disclosed in WO 00/22103 and variants at least 95% thereto.

Amylases further may be from Bacillus sp. A 7-7 (DSM 12368) having an amino acid sequence at least 95% identical to SEQ ID NO:2, in particular over the region of the amino acids 32 to 516 according to SEQ ID NO:2, as disclosed in WO 02/10356.

Amylases further may be from Bacillus strain TS-23 having SEQ ID NO:2 as disclosed in WO 2009/061380 and variants thereof.

Amylases further may be from Cytophaga sp. having SEQ ID NO:1 as disclosed in WO 2013/184577 and variants at least 95% thereto.

Amylases further may be from Bacillus megaterium DSM 90 having SEQ ID NO: 1 as disclosed in WO 2010/104675 and variants at least 95% thereto.

Amylases further may be from Bacillus sp. comprising amino acids 1 to 485 of SEQ ID NO:2 as described in WO 00/60060 and variants at least 95% thereto.

Amylases further may be from Bacillus amyloliquefaciens or variants thereof, preferably selected from amylases according to SEQ ID NO: 3 as described in WO 2016/092009.

Amylases may have SEQ ID NO: 12 as described in WO 2006/002643 or amylase variants thereof comprising the substitutions Y295F and M202LITV within said SEQ ID NO:12.

Amylases may have SEQ ID NO:6 as described in WO 2011/098531 or amylase variants comprising a substitution at one or more positions selected from the group consisting of 193 [G,A, S, T or M], 195 [F,W,Y, L,I or V], 197 [F, W, Y, L,I or V], 198 [Q or N], 200 [F,W, Y,L,I or V], 203 [F, W, Y, L,I or V], 206 [F,W,Y,N,L,I,V,H,Q,D or E], 210 [F,W, Y, L,I or V], 212 [F,W, Y,L,I or V], 213 [G,A,S, T or M] and 243 [F,W,Y,L,I or V] within said SEQ ID NO:6.

Amylases may have SEQ ID NO: 1 as described in WO 2013/001078 or amylase variants comprising an alteration at two or more (several) positions corresponding to positions G304, W140, W189, D134, E260, F262, W284, W347, W439, W469, G476, and G477 within said SEQ ID NO: 1.

Amylases may have SEQ ID NO:2 as described in WO 2013/001087 or amylase variants comprising a deletion of positions 181+182, or 182+183, or 183+184, within said SEQ ID NO:2, optionally comprising one or two or more modifications in any of positions corresponding to W140, W159, W167, Q169, W189, E194, N260, F262, W284, F289, G304, G305, R320, W347, W439, W469, G476 and G477 within said SEQ ID NO:2.

Amylases may be hybrid alpha-amylases from above mentioned amylases as for example as described in WO 2006/066594.

Hybrid amylases may be according to WO 2014/183920 with A and B domains having at least 90% identity to SEQ ID NO:2 of WO 2014/183920 and a C domain having at least 90% identity to SEQ ID NO:6 of WO 2014/183920, wherein the hybrid amylase has amylolytic activity; preferably the hybrid alpha-amylase is at least 95% identical to SEQ ID NO: 23 of WO 2014/183920 and having amylolytic activity.

Hybrid amylases may be according to WO 2014/183921 with A and B domains having at least 75% identity to SEQ ID NO: 2, SEQ ID NO: 15, SEQ ID NO: 20, SEQ ID NO: 23, SEQ ID NO: 29, SEQ ID NO: 26, SEQ ID NO: 32, and SEQ ID NO: 39 as disclosed in WO 2014/183921 and a C domain having at least 90% identity to SEQ ID NO: 6 of WO 2014/183921, wherein the hybrid amylase has amylolytic activity; preferably, the hybrid alpha-amylase is at least 95% identical to SEQ ID NO: 30 as disclosed in WO 2014/183921 and having amylolytic activity; Hybrid amylases may be according to WO 2021/032881 comprising an A and B domain originating from the alpha amylase originating from Bacillus sp. A 7-7 (DSM 12368) and a C domain originating from the alpha-amylase from Bacillus cereus; preferably, the A and B domain are at least 75% identical to the amino acid sequence of SEQ ID NO: 42 and a C domain is at least 75% identical to the amino acid sequence of SEQ ID NO: 44-both sequences as disclosed in WO 2021/032881; more preferably, the hybrid amylase is at least 80% identical to SEQ ID NO: 54 as disclosed in WO 2021/032881.

In one embodiment, at least one amylase is selected from commercially available amylases which include but are not limited to products sold under the trade names Duramyl™, Termamyl™, Fungamyl™, Stainzyme™, Stainzyme Plus™, Natalase™, Liquozyme X and BAN™ Amplify™, Amplify Prime™ (from Novozymes A/S), and Rapidase™, Purastar™, Powerase™, Effectenz™ (M100 from DuPont), Preferenz™ (S1000, S110 and F1000; from DuPont), Prima-Green™ (ALL; DuPont), Optisize™ (DuPont).

### Mannanases

“Mannanase” as described herein are enzymes selected from the group of mannan degrading enzyme. The mannan degrading enzyme may be selected from β-mannosidase (EC 3.2.1.25), endo-1,4-β-mannosidase (EC 3.2.1.78), and 1,4-β-mannobiosidase (EC 3.2.1.100). Preferably, the mannan degrading enzyme is selected from the group of endo-1,4-β-mannosidase (EC 3.2.1.78), a group of enzymes which may be called endo-β-1,4-D-mannanase, β-mannanase, or mannanase herein.

The mannanase may be selected from alkaline mannanase of Family 5 or 26 (i.e., GH5 or GH26). The term “alkaline mannanase” is meant to encompass mannanases having an enzymatic activity of at least 40% of its maximum activity at a given pH ranging from 7 to 12, preferably 7.5 to 10.5.

The mannanase may be selected from mannanases originating from Bacillus organisms, such as described in JP-0304706 [beta-mannanase from Bacillus sp.], JP-63056289 [alkaline, the mostable beta-mannanase], JP-63036774 [Bacillus microorganism FERM P-8856 producing beta-mannanase and beta-mannosidase at an alkaline pH], JP-08051975 [alkaline beta-mannanases from alkalophilic Bacillus sp. AM-001], WO 97/11164 [mannanase from Bacillus amylo-liquefaciens], WO 91/18974 [mannanase active at an extreme pH and temperature], WO 97/11164 [mannanase from Bacillus amyloliquefaciens], WO 2014/100018 [endo-(3-mannanase 1 cloned from a Bacillus circulans or Bacillus lentus strain CMG1240 (Bleman1; see U.S. Pat. No. 5,476,775)]. Suitable mannanases are described in WO 99/064619].

The mannanase may be selected from mannanases originating from Trichoderma organisms, such as disclosed in WO 93/24622.

The mannanase may be selected from a commercially available mannanase such as Manna-way® (Novozymes A/S) or Preferenz® (M100) (DuPont).

### Cellulases

“Cellulases” are enzymes capable of hydrolysing of cellulose. Cellulases may be selected from cellobiohydrolase (1,4-P-D-glucan cellobiohydrolase, EC 3.2.1.91), endo-ss-1,4-glucanase (EC 3.2.1.4) and ss-glucosidase (EC 3.2.1.21). Endoglucanases of EC class 3.2.1.4 may be named endoglucanase, endo-1,4-ss-D-glucan 4-glucano hydrolase, endo-1,4-beta-glucanase, carboxymethyl cellulase, and beta-1,4-glucanase.

Endoglucanases may be classified by amino acid sequence similarities (Henrissat, B. Accessed at UniProt Oct. 26, 2011) under family 5 containing more than 20 endoglucanases of EC 3.2.1.4. Reference is also made to T.-M. Enveri, “Microbial Cellulases” in W.M. Fogarty, Microbial Enzymes and Biotechnology, Applied Science Publishers, p. 183-224 (1983); Methods in Enzymol-ogy, (1988) Vol. 160, p. 200-391 (edited by Wood, W.A. and Kellogg, S.T.); Béguin, P., “Molecular Biology of Cellulose Degradation”, Annu. Rev. Microbiol. (1990), Vol. 44, pp. 219248; Begun, P. and Aubert, J-P., “The biological degradation of cellulose”, FEMS Microbiology Reviews 13 (1994) p.25-58; Henrissat, B., “Cellulases and their interaction with cellulose”, Cellulose (1994), Vol. 1, pp. 169-196.

Preferably, at least one cellulase is selected of the glycosyl hydrolase family 7 (GH7, pfam00840), preferably selected from endoglucanases (EC 3.2.1.4).

Preferably, an alkaline cellulase is used, wherein “alkaline cellulase” is meant to encompass cellulases having enzymatic activity at a given pH ranging from 7 to 12, preferably 7.5 to 10.5.

In one embodiment, the cellulase is selected from cellulases comprising a cellulose binding domain. In one another embodiment, the cellulase comprises a catalytic domain, but no cellulose binding domain.

In one embodiment, at least one endoglucanases of EC class 3.2.1.4 is originating from
Bacillus, such as Bacillus sp. CBS 670.93 and CBS 669.93
Melanocarpus, such as Melanocarpus albomyces as disclosed in WO 97/14804
Clostridium, e.g. Clostridium thermocellum
Humicola, such as Humicola insolens (DSM1800) as disclosed in EP 0495257, EP 0531315, EP 0531372, U.S. Pat. Nos. 4,435,307, 5,648,263, 5,776,757, WO 89/09259, WO 91/17244, WO 94/07998 (sequence displayed in FIG. 1 43kd human variants thereof), WO 95/24471, WO 96/11262 and WO 98/12307.
Fusarium, such as Fusarium oxysporum e.g. strain J79 (DSM2672) as disclosed in EP 0495257, EP 0531315, EP 0531372, U.S. Pat. Nos. 5,648,263, 5,776,757, WO 89/09259, WO 91/17244, WO 95/24471 and WO 96/11262
Thielavia, such as Thielavia terrestris or Myceliophthora thermophila strain CBS 11765 as disclosed in EP 0531315, U.S. Pat. Nos. 5,648,263, 5,776,757, WO 89/09259, WO 91/17244, WO 95/24471, WO 96/11262, WO 96/29397 (SEQ ID NO: 9 and variants thereof), and WO 98/12307.
Trichoderma, such as Trichoderma reesei, Trichoderma longibrachiatum or Trichoderma harzianum as disclosed in EP 1305432, EP 1240525, WO 92/06165, WO 94/21801, WO 94/26880, WO 95/02043, WO 95/24471 and WO 02/099091.
Aspergillus, such as Aspergillus aculeatus as disclosed in WO 93/17244
Erwinia, such as Erwinia chrysanthermi as described by M. H. Boyer et. al. in European Journal of Biochemistry, vol. 162, page 311-316 (1987).
Acremonium such as Acremonium sp., Acremonium persicinum, Acremonium acremonium, Acremonium brachypenium, Acremonium dichromosporum, Acremonium obclavatum, Acremonium pinkertoniae, Acremonium roseogriseum, Acremonium incoloratum, and Acremonium furatum as disclosed in WO 96/11262 and WO 96/29397 (SEQ ID NO: 5 and variants thereof).
Cellvibrio such as Cellvibrio mixtus DSM 11683, Cellvibrio mixtus DSM 11684, Cellvibrio mixtus DSM 11685, Cellvibrio mixtus ACM 2601, Cellvibrio mixtus DSM 1523, and Cellvibrio gilvus DSM 11686, as disclosed in WO 98/08940.
Cephalosporium, such as Cephalosporium sp. RYM-202 as disclosed in WO 96/11262.

Suitable cellulases include also those, which are variants of the above described cellulases which have cellulolytic activity. In one embodiment cellulase variants include variants with at least 40 to 100% identity when compared to the full length polypeptide sequence of the parent enzyme as disclosed above. In one embodiment cellulase variants having cellulolytic activity are at least 70%, at least 75%, at least 80%, at least 85%, at least 90%, at least 91%, at least 92%, at least 93%, at least 94%, at least 95%, at least 96%, at least 97%, at least 98% or at least 99% similar and/or identical to the full length polypeptide sequence of the parent enzyme as disclosed above.

The cellulase may be a Humicola insolens DSM 1800 cellulase complex having endoglucanase, cellobiohydrolase and beta-glucosidase activity.

The cellulase may be a Humicola insolens DSM 1800 endoglucanase (EC 3.2.1.4), preferably having the polypeptide sequence according to position 21-435 of SEQ ID NO:2 as disclosed in WO 2018/224544 or variants at least 95% identical thereto.

The cellulase may be a Humicola insolens endoglucanase (EC 3.2.1.4) having 43 kD, preferably according to the polypeptide sequence as disclosed in FIG. 1a of WO 94/07998 (“43kDhum”) or variants thereof which are preferably at least 90% identical thereto, preferably those disclosed in WO 94/07998.

The cellulase may be a Bacillus sp. cellulase (EC 3.2.1.4) selected from a polypeptide at least 80% similar and/or identical to the amino acid sequence of position 1 to position 773 of SEQ ID NO: 2 of WO 2004/053039 or a catalytically active fragment thereof. In one embodiment, the cellulase is a mature polypeptide which is at least 95% identical to SEQ ID NO:1 of WO 2018/224544.

The cellulase may be a Thielavia terrestris cellulase (EC 3.2.1.4) having a polypeptide at least 80% similar and/or identical to the amino acid sequence of position 1 to position 299 of SEQ ID NO: 4 of WO 2004/053039 or a catalytically active fragment thereof. In one embodiment, the cellulase is a mature polypeptide which is at least 95% identical to SEQ ID NO:4 of WO 2018/224544.

The cellulase may be a mature Sordaria fimicola cellulase, preferably having a polypeptide sequence according to SEQ ID NO:5 of WO 2018/224544 or variants at least 95% identical thereto.

At least one cellulase may be selected from Renozyme®, Celluzyme®, Celluclean®, Endolase® and Carezyme® (Novozymes A/S), Clazinase™, and Puradax HA™ (Genencor Int. Inc.), and KAC-500 (B) TM (Kao Corporation).

### Detergent Compositions

In one embodiment, the present invention is directed to the use of the amylase variant in a detergent composition. Thus, the present invention is also directed to a detergent composition comprising the amylase variant described herein and one or more detergent component.

Thus, the present invention therefore also refers to a method for making a detergent composition comprising the steps of mixing
a) an amylase variant as described herein; and
b) one or more detergent component described herein.

Further, the present invention therefore also refers to a method for making a detergent composition with improved amylase stability and/or for providing a detergent composition with improved wash performance comprising the steps of mixing
c) an amylase variant as described herein; and
d) one or more detergent component described herein.

The one or more detergent component may be selected from the group consisting of additional enzyme different from the amylase variant, enzyme stabilizing system, surfactant, defoamer, builder, polymer, bleaching system (bleach), rheology modifier, hydrotrope, softening agent, desiccant, whitening agent, buffer, preservative, anti-corrosion additive, dyestuff and fragrance. Preferably, at least one component of the detergent is selected from the group consisting of surfactant, builder, polymer, preservative, and second enzyme different to the amylase variant.

Preferably one or more of the detergent component, preferably the surfactant and/or the builder, is bio-degradable and/or bio-based.

Detergent components may have more than one function in the final application of a detergent composition, therefore any detergent component mentioned in the context of a specific function herein, may also have another function in the final application of a detergent composition. The function of a specific detergent component in the final application of a detergent composition usually depends on its amount within the detergent composition, i.e., the effective amount of a detergent component. Detergent components vary in type and/or amount in a detergent composition depending on the desired application such as laundering white textiles, colored textiles, and wool. The component(s) chosen further depend on physical form of a detergent composition (liquid, solid, gel, provided in pouches or as a tablet, etc.). The component(s) chosen e.g. for laundering formulations further depend on regional conventions which themselves are related to aspects like washing temperatures used, mechanics of laundry machine (vertical vs. horizontal axis machines), water consumption per wash cycle etc. and geographical characteristics like average hardness of water.

In one embodiment, a detergent composition is a formulation of more than two detergent components, wherein at least one component is effective in stain-removal, at least one component is effective in providing the optimal cleaning conditions, and at least one component is effective in maintaining the physical characteristics of the detergent.

The detergent composition can be a liquid or solid detergent composition or a combination of liquid and solid detergent composition. The liquid detergent composition is preferably a gel detergent composition. The solid detergent composition can be a soap bar or a powder detergent composition, preferably a powder detergent composition, wherein the powder detergent composition can be pressed to a tablet.

The detergent composition can be a unit dose or multi-unit dose composition. The detergent composition can be in the form of a pouch, including multi-compartment pouches. The detergent composition can be a laundry or dish washing detergent composition, suitable for home care and/or industrial and institutional (I&I) cleaning. Both laundry and dish wash composition can be in the form of a hand wash or automated wash composition. Preferably the dish wash composition is an Automatic Dish Wash (ADW).

Detergent pouches can be of any form, shape and material which is suitable for holding the composition, e.g., without allowing the release of the composition from the pouch prior to water contact. The pouch is made from water-soluble film, which encloses an inner volume. Said inner volume can be divided into compartments of the pouch. Preferred films are polymeric materials preferably polymers which are formed into a film or sheet, e.g., polyvinyl alcohol copolymers and hydroxypropyl methyl cellulose (HPMC). The pouches can comprise a solid laundry detergent composition or part components and/or a liquid detergent composition or part components separated by the water-soluble film. The compartment for liquid components can be different in composition from compartments containing solids (see e.g. US 2009/0011970).

Preferably, the amylase variant according to the present invention may be added to a detergent composition in an amount corresponding to 0.002 to 6 mg of active enzyme variant per g of detergent composition, preferably, 0.005-5 mg/g, 0.005-3 mg/g, 0.01-2 mg/g, or 0.05-2 mg/g. In one embodiment, the detergent composition has a pH in the range of 5-12, preferably in the range of 6-11, more preferably in a range selected from 6-10, 7-9, and 7.5-8.5. In one embodiment, the formulation is a detergent composition, preferably a liquid detergent composition. In one embodiment, the detergent compositions according to the invention comprise one or more surfactant(s). According to its ionic charge, a surfactant is called non-ionic, anionic, cationic, or amphoteric.

The detergent composition of the present invention may comprise one or more surfactants, which may be anionic and/or cationic and/or non-ionic and/or semi-polar and/or zwitterionic, or a mixture thereof. In preferred embodiments, the detergent compositions of the invention comprise at least one surfactant. In a particular embodiment, the detergent composition of the present invention includes a mixture of one or more nonionic surfactants and one or more anionic surfactants. The surfactant(s) is/are typically present at a level of from about 0.1 to 60 wt.-%, such as 1 to 40 wt.-%, 3 to 20 wt.-% or 3 to 10 wt.-%. The surfactant(s) is/are chosen based on the desired cleaning application, and includes any conventional surfactant(s) known in the art. Any surfactant known in the art for use in detergents may be utilized. Non-limiting examples of surfactants are disclosed Mccutcheon's 2016 Detergents and Emulsifiers, and Mccutcheon's 2016 Functional Materials, both North American and International Edition, MC Publishing Co, 2016 edition. Further useful examples are disclosed in earlier editions of the same publications which are known to those skilled in the art.

When included therein, the detergent will usually comprise from about 1 to 40 wt.-%, such as 5 to 30 wt.-%, 5 to 15 wt.-% or 20 to 25 wt.-%, of an anionic surfactant. Non-limiting examples of anionic surfactants include sulfates and sulfonates, in particular linear alkyl benzene sulfonates (LAS), isomers of LAS, branched alkyl benzene sulfonates (BABS), phenyl alkane sulfonates, alpha-olefin sulfonates (AOS), olefin sulfonates, alkene sulfonates, alkane-2,3-diylbis(sulfates), hydroxy alkane sulfonates and disulfonates, alkyl sulfates (AS) such as sodium dodecyl sulfate (SDS), fatty alcohol sulfates (FAS), primary alcohol sulfates (PAS), alcohol ether sulfates (AES or AEOS or FES, also known as alcohol ethoxy sulfates or fatty alcohol ether sulfates), secondary alkane sulfonates (SAS), paraffin sulfonates (PS), ester sulfonates, sulfonated fatty acid glycerol esters, alpha-sulfo fatty acid methyl esters (alpha-SFMe or SES) including methyl ester sulfonate (MES), alkyl- or alkenyl succinic acid, dodecenyl/tetradecenyl succinic acid (DTSA), fatty acid derivatives of amino acids, diesters and monoesters of sulfo succinic acid or soap, and combinations thereof.

When included therein, the detergent will usually comprise from about 0 to 10 wt.-% of a cationic surfactant. Non-limiting examples of cationic surfactants include alkyl dimethyl ethanolamine quat (ADMEAQ), cetyl trimethyl ammonium bromide (CTAB), dimethyl distearyl ammonium chloride (DSDMAC), and alkyl benzyl dimethyl ammonium, alkyl quaternary ammonium compounds, alkoxylated quaternary ammonium (AQA) compounds, and combinations thereof. When included therein, the detergent will usually comprise from about 0.2 to 40 wt.-% of a nonionic surfactant, e.g. 0.5 to 30 wt.-%, in particular 1 to 20 wt.-%, 3 to 10 wt.-%, 3 to 5 wt.-% or 8 to 12 wt.-%. Non-limiting examples of non-ionic surfactants include alcohol ethoxylates (AE or AEO), alcohol propoxylates, propoxylated fatty alcohols (PFA), alkoxylated fatty acid alkyl esters, such as ethoxylated and/or propoxylated fatty acid alkyl esters, alkyl phenol ethoxylates (APE), nonyl phenol ethoxylates (NPE), alkyl polyglycosides (APG), alkoxylated amines, fatty acid monoethanolamides (FAM), fatty acid diethanol amides (FADA), ethoxylated fatty acid monoethanolamides (EFAM), propoxylated fatty acid monoethanolamides (PFAM), polyhydroxy alkyl fatty acid amides, or N-acyl N-alkyl derivatives of glucosamine (glucamides, GA, or fatty acid glucamide, FAGA), as well as products available under the trade names SPAN and TWEEN, and combinations thereof.

When included therein, the detergent will usually comprise from about 0 to 10 wt.-% of a semi-polar surfactant. Non-limiting examples of semipolar surfactants include amine oxides (AO) such as alkyl dimethyl amine oxide, N-(coco alkyl)-N, N-dimethyl amine oxide and N-(tallow-al-kyl)-N, N-bis-(2-hydroxy ethyl)amine oxide, fatty acid alkanol amides and ethoxylated fatty acid alkanol amides, and combinations thereof.

When included therein, the detergent will usually comprise from about 0 to 10 wt.-% of a zwitterionic surfactant. Non-limiting examples of zwitterionic surfactants include betaine, alkyl dimethyl betaine, sulfo betaine, and combinations thereof.

The detergent compositions according to the invention may comprise one or more compounds selected from complexing agents (chelating agents (chelants), sequestrating agents), precipitating agents, and ion exchange compounds which may form water-soluble complexes with calcium and magnesium. Such compounds may be called “builders” or “building agents” herein, without meaning to limit such compounds to this function in the final application of a detergent composition.

In one embodiment, the detergent composition of the invention comprises at least one builder selected from non-phosphate based builders such as sodium gluconate, citrate(s), silicate(s), carbonate(s), phosphonate(s), amino carboxylate(s), polycarboxylate(s), polysulfonate(s), and polyphosphonate(s). In one embodiment, the detergent composition of the invention comprises a strong sequestering builder. Preferably, detergent compositions of the current invention are free from phosphate, meaning essentially free from phosphate-based builders. Herein, “essentially free from phosphate” is to be understood as meaning that the content of phosphate and polyphosphate is in sum in the range of 10 ppm to 1% by weight, determined by gravimetry and referring to the respective inventive detergent composition. In another preferred embodiment, the detergent composition comprises phosphonate, wherein the phosphonate is preferably DTPMP and/or HEDP.

In one embodiment, the detergent compositions of the invention comprise at least one “citrate” selected from the mono- and the dialkali metal salts and in particular the mono- and preferably the trisodium salt of citric acid, ammonium or substituted ammonium salts of citric acid as well as citric acid as such. Citrate can be used as the anhydrous compound or as the hydrate, for example as sodium citrate dihydrate. The citrate may be comprised in a total amount in the range of 0% to about 20% by weight, in the range of about 0.5% to about 10% by weight, or in the range of 1-5% by weight, all relative to the total weight of the detergent composition. In one embodiment, the detergent composition of the invention comprises a total amount of citrate in the range of about 1-3% relative to the total weight of the detergent composition.

Detergent compositions of the invention may comprise one or more silicates. “Silicate(s)” in the context of the present invention include in particular sodium disilicate and sodium metasilicate, aluminosilicates such as sodium aluminosilicates like zeolith A (i.e. Na₁₂ (AlO₂)₁₂ (SiO₂)₁₂*27H₂O), and sheet silicates, in particular those of the formula alpha —Na₂Si₂O₅, beta-Na₂Si₂O₅, and delta-Na₂Si₂O₅.

Detergent compositions of the invention may comprise one or more carbonates. The term “carbonate(s)” includes alkali metal carbonates and alkali metal hydrogen carbonates, preferred are the sodium salts. Particularly suitable is sodium carbonate (Na₂CO₃).

Detergent compositions of the invention may comprise one or more phosphonates. “Phosphonates” include, but are not limited to 2-phosphinobutane-1,2,4-tricarboxylic acid (PBTC); ethylenediaminetetra(methylenephosphonic acid) (EDTMPA); 1-hydroxyethane-1,1-diphosphonic acid (HEDP), CH₂C(OH)[PO(OH)₂]₂; aminotris(methylenephosphonic acid) (ATMP), N[CH₂PO(OH)₂]₃; aminotris(methylenephosphonate), sodium salt (ATMP), N[CH₂PO(ONa) 2]3; 2-hydroxyethyliminobis(methylenephosphonic acid), HOCH₂CH₂N[CH₂PO(OH)₂]₂; diethylenetriaminepenta(methylenephosphonic acid) (DTPMP), (HO)₂POCH₂N[CH₂CH₂N[CH₂PO(OH)₂]₂]₂; diethylenetriaminepenta(methylenephosphonate), sodium salt, C₉H₍₂₈₋ₓ₎ N₃NaₓO₁₅P₅ (x=7); hexamethylenediamine (tetramethylenephosphonate), potassium salt, C₁₀H₍₂₈₋ₓ₎ N₂Kx012P₄ (x=6); and bis(hexamethylene)triamine (pentamethylenephosphonic acid), (HO₂)POCH₂N[(CH₂)₂N[CH₂PO(OH)₂]₂]₂. Salts thereof may be suitable, too.

Detergent compositions of the invention may comprise one or more aminocarboxylates. Non-limiting examples of suitable “amino carboxylates” include, but are not limited to: diethanol glycine (DEG), dimethylglycine (DMG), nitrilitriacetic acid (NTA), N-hydroxyethylaminodiacetic acid, ethylenediaminetetraacetic acid (EDTA), N-(2hydroxyethyl)iminodiacetic acid (HEIDA), hydroxy-ethylenediaminetriacetic acid, N-hydroxyethyl-ethylenediaminetriacetic acid (HEDTA), hydroxy-ethylenediaminetetraacetic acid, diethylenetriaminepentaacetic acid (DTPA), and methylgly-cinediacetic acid (MGDA), glutamic acid-diacetic acid (GLDA), iminodisuccinic acid (IDS), hy-droxyiminodisuccinic acid, ethylenediaminedisuccinic acid (EDDS), aspartic acid-diacetic acid, and alkali metal salts or ammonium salts thereof. Further suitable are aspartic acid-N-monoace-tic acid (ASMA), aspartic acid-N,N-diacetic acid (ASDA), aspartic acid-N-monopropionic acid (ASMP), N-(2-sulfomethyl) aspartic acid (SMAS), N-(2-sulfoethyl) aspartic acid (SEAS), N-(2-sulfomethyl) glutamic acid (SMGL), N-(2-sulfoethyl) glutamic acid (SEGL), N-methyliminodiace-tic acid (MIDA), alpha-alanine-N,N-diacetic acid (alpha-ALDA), serine-N, N-diacetic acid (SEDA), isoserine-N, N-diacetic acid (ISDA), phenylalanine-N, N-diacetic acid (PHDA), anthranilic acid-N, N-diacetic acid (ANDA), sulfanilic acid-N, N-diacetic acid (SLDA), taurine-N, N-diacetic acid (TUDA) and sulfomethyl-N,N-diacetic acid (SMDA) and alkali metal salts or ammonium salts thereof. Preferred are MGDA or EDDS. The term “ammonium salts” as used in in this context refers to salts with at least one cation that bears a nitrogen atom that is permanently or temporarily quaternized. Examples of cations that bear at least one nitrogen atom that is permanently quaternized include tetramethylammonium, tetraethylammonium, dimethyldiethyl ammonium, and n-C₁₀-C₂₀-alkyl trimethyl ammonium. Examples of cations that bear at least one nitrogen atom that is temporarily quaternized include protonated amines and ammonia, such as monomethyl ammonium, dimethyl ammonium, trimethyl ammonium, monoethyl ammonium, di-ethyl ammonium, triethyl ammonium, n-C₁₀-C₂₀-alkyl dimethyl ammonium 2-hydroxyethylammonium, bis(2-hydroxyethyl) ammonium, tris(2-hydroxyethyl) ammonium, N-methyl 2-hydroxyethyl ammonium, N, N-dimethyl-2-hydroxyethylammonium, and especially NH₄⁺.

In one embodiment, detergent compositions of the invention comprise more than one builder. Preferably, inventive detergent compositions contain less than 0.2% by weight of nitrilotriacetic acid (NTA), or 0.01 to 0.1% NTA by weight relative to the total weight of the detergent composition.

In one embodiment, the detergent composition of the invention comprises of at least one aminocarboxylate selected from methylglycine diacetate (MGDA), glutamic acid diacetate (GLDA), and the respective salts thereof, e.g., alkali (such as sodium) salts thereof in amounts in the range of 0.1% to 25.0% by weight, in the range of 1.0% to 18.0% by weight, in the range of 3.0% to 15.0% by weight, in the range of 3.0% to 10.0% by weight, or in the range of 5.0% to 8.0% by weight relative to the total weight of the detergent composition.

The detergent compositions of the invention may comprise one or more hydrotropes. One or more hydrotropes may be selected from organic solvents such as ethanol, isopropanol, ethylene glycol, 1,2-propylene glycol, and further organic solvents known in the art that are water-miscible under normal conditions without limitation. In one embodiment, the detergent composition of the invention comprises 1,2-propylene glycol in a total amount in the range of 5-10% by weight, preferably of about 6% by weight, all relative to the total weight of the detergent composition. Further non-limiting examples of hydrotropes include sodium benzene sulfonate, sodium p-tolu-ene sulfonate (STS), sodium xylene sulfonate (SXS), sodium cumene sulfonate (SCS), sodium cymene sulfonate, amine oxides, alcohols and polyglycol ethers, sodium hydroxy naphthoate, sodium hydroxy naphthalene sulfonate, sodium ethyl hexyl sulfate, and combinations thereof.

In one embodiment, the detergent composition comprises at least one preservative. Preferably, preservative means substances that are added to a liquid composition for the purpose of preservation, meaning more preferably that compounds known to have preserving features comprised in a liquid composition formed in the production process are excluded from the term preservatives. In one embodiment, the preservative is selected from the group consisting of 2-phenoxyethanol, glutaraldehyde, 2-bromo-2-nitropropane-1,3-diol, and formic acid in acid form or as its salt, and 4,4′-dichloro 2-hydroxydiphenylether. Usually, the liquid compositions of the invention comprise at least one preservative in amounts below 10 ppm, such as in amounts ranging from 2 ppm to 5% by weight relative to the total weight of the liquid composition. Alternatively, the detergent composition is free from preservatives, meaning that preservatives are comprised in amounts less than 1 ppm, preferably below 0 ppm.

In one embodiment, the detergent composition comprising an amylase variant as described herein further comprises one or more second enzyme different from the amylase variant. Preferably, the second enzyme is selected from the group consisting of, proteases, second amylases, lipases, cellulases, mannanases, hemicellulases, phospholipases, esterases, pectinases, lactases, peroxidases, xylanases, cutinases, pectate lyases, keratinases, reductases, oxidases, phenoloxidases, lipoxygenases, ligninases, pullulanases, tannases, pentosanases, malanases, beta-glucanases, arabinosidases, hyaluronidases, chondroitinases, laccases, nucleases, DNase, phosphodiesterases, phytases, carbohydrases, galactanases, xanthanases, xyloglucanases, oxidoreductase, perhydrolases, aminopeptidase, asparaginase, carbohydrase, carboxypeptidase, catalase, chitinase, cyclodextrin glycosyltransferase, alpha-galactosidase, beta-galactosidase, glucoamylase, alpha-glucosidase, beta-glucosidase, invertase, ribonuclease, transglutaminase, and dispersins, and combinations of at least two of the foregoing types. More preferably, the second enzyme is selected from the group consisting of protease, lipases, cellulases, mannanases, xylanases, DNases, dispersins, pectinases, oxidoreductases, and cutinases, and combinations of at least two of the foregoing types. Most preferably, the second enzyme is protease, preferably, subtilisin protease.

Particular preferred additional enzymes are disclosed elsewhere herein and that description is incorporated by reference also to this part of the description.

The composition of the present invention can comprise one type of enzyme or more than one enzyme of different types, e.g., an amylase and a protease, or more than one enzyme of the same type, e.g., two or more different proteases, or mixtures thereof, e.g., an amylase and two different proteases.

The detergent compositions may comprise water-soluble sources of calcium and/or magnesium ions. In one embodiment, at the detergent composition comprises an enzyme stabilizing system as described herein. Preferably, in particular in the case of liquid detergent compositions, the detergent composition may comprise at least one protease inhibitor as described herein, preferably selected from boronic acid derivatives, preferably 4-FPBA, and peptide aldehyde, preferably Z-VAL-H or Z-GAY-H. Preferably, the detergent composition is boron-free.

In one embodiment, the invention relates to a method to provide a detergent composition, preferably a liquid detergent composition, more preferably a liquid laundering detergent composition, comprising the steps of mixing in one or more steps
(a) at least one amylase variant according to the invention, preferably wherein the amylase is provided within an amylase variant formulation as described herein; and
(b) at least one detergent component, preferably selected from surfactant, builder, polymer, preservative, and second enzyme different to the amylase variant, present in amounts effective in cleaning performance and/or effective in maintaining the physical characteristics of the detergent.

In one embodiment, the present invention is directed to a detergent composition comprising
a) an amylase variant as described herein;
b) one or more surfactant, preferably, in a concentration of 0.2-65%, preferably 0.2-40%,
c) one or more builder, preferably, in a concentration of 0.01-25%, and
d) optionally one or more additional compound selected from the group consisting of additional enzyme different from the amylase under a), defoamer, polymer, bleaching system (bleach), rheology modifier, hydrotrope, softening agent, desiccant, whitening agent, buffer, preservative, anti-corrosion additive, dyestuff and fragrance; preferably wherein detergent composition, is a liquid, powder, pouch, or capsule detergent composition.

Preferably the detergent composition, preferably powder detergent composition, of the present invention comprises in addition to the amylase variant as described herein one or more of the compounds selected from the group consisting of alcohol ethoxylate 7EO, Coco fatty acid C₁₂-18, C₁₂-C₁₄-fatty alcohol ether sulfate (1-3 EO, preferably 2 EO), Linear alkyl benzene sulphonic acid, AcetateNa, CitrateNa, Na Silicate, Na Carbonate, Na Phospahte, Na Hydrogencarbonate, Zeolite4A, HEDP, MGDA, Na Sulfate, Na Chloride, optical brightener, and polymers and optionally Bleach activator and Percarbonate.

Preferably the detergent composition, preferably powder detergent composition, of the present invention comprises in addition to the amylase variant as described herein
b) one or more surfactant selected from the group consisting of Alcohol ethoxylate 7EO, Coco fatty acid C12-18, C12-C14-fatty alcohol ether sulfate (1-3 EO, preferably 2 EO), Linear alkyl benzene sulphonic acid, preferably, in a concentration of 0.2-65%,
c) one or more builder selected from the group consisting of HEDP, MGDA, GLDA, and DTPMP, preferably, in a concentration of 0.01-25%, and
d) one or more compound selected from the group consisting of AcetateNa, CitrateNa, Na Silicate, Na Carbonate, Na Phospahte, Na Hydrogencarbonate, Zeolite4A, Na Sulfate, Na Chloride, optical brightener, and polymers, and optionally Bleach activator and Percarbonate.

Preferably the detergent composition, preferably liquid detergent composition, of the present invention comprises in addition to the amylase variant as described herein one or more of the compounds selected from the group consisting of alcohol ethoxylate 7EO, Coco fatty acid C12-18, C12-C14-fatty alcohol ether sulfate (1-3 EO, preferably 2 EO), Linear alkyl benzene sulphonic acid, sulphonic acid, 1,2 Propandiol, Triethanolamine, Monoethanolamine, NaOH, Glycerol, Ethanol, Na citrate, and Polymer.

Preferably the detergent composition, preferably liquid detergent composition, of the present invention comprises in addition to the amylase variant as described herein
b) one or more surfactant selected from the group consisting of Alcohol ethoxylate 7EO, Coco fatty acid C12-18, C12-C14-fatty alcohol ether sulfate (1-3 EO, preferably 2 EO), Linear alkyl benzene sulphonic acid, preferably, in a concentration of 0.2-65%,
c) one or more builder selected from the group consisting of HEDP, MGDA, GLDA, and DTPMP, preferably, in a concentration of 0.01-25%, and
d) one or more compound selected from the group consisting of sulphonic acid, 1,2 Propandiol, Triethanolamine, Monoethanolamine, NaOH, Glycerol, Ethanol, Na citrate, and Polymer.

In one embodiment, the amylase variant described herein is included in a detergent composition comprising one or more, preferably all, compounds selected from the group consisting of (all percentages are w/w):

A formulation comprising the amylase variant described herein, from 0.05% to 1.0%; Anionic detersive surfactant (such as alkyl benzene sulphonate, alkyl ethoxylated sulphate and mixtures), from 8% to 15%; Non-ionic detersive surfactant (such as alkyl ethoxylated alcohol), from 0.5% to 4%;
Cationic detersive surfactant (such as quaternary ammonium compounds), from 0 to 4%;
Other detersive surfactant (such as zwitterionic detersive surfactants, amphoteric surfactants and mixtures thereof), from 0% to 4%;
Carboxylate polymer (such as co-polymers of maleic acid and acrylic acid), from 1% to 4%;
Polyethylene glycol polymer (such as a polyethylene glycol polymer comprising poly vinyl acetate side chains), from 0.5% to 4%;
Polyester soil release polymer (such as Repel-o-tex from and/or Texcare polymers), from 0.1 to 2%;
Cellulosic polymer (such as carboxymethyl cellulose, methyl cellulose and combinations thereof), from 0.5% to 2%;
Other polymer (such as amine polymers, dye transfer inhibitor polymers, hexamethylenediamine derivative polymers, and mixtures thereof), from 0% to 4%;
Zeolite builder and phosphate builder (such as zeolite 4A and/or sodium tripolyphosphate), from 0% to 4 wt %;
Other builder (such as sodium citrate and/or citric acid), from 0% to 3%;
Carbonate salt (such as sodium carbonate and/or sodium bicarbonate), from 15% to 30%;
Silicate salt (such as sodium silicate), from 0% to 10%;
Filler (such as sodium sulphate and/or bio-fillers), from 10% to 40%;
Source of available oxygen (such as sodium percarbonate), from 10% to 20%;
Bleach activator (such as tetraacetylethylene diamine (TAED) and/or nonanoyloxybenzenesulphonate (NOBS), from 2% to 8%;
Bleach catalyst (such as oxaziridinium-based bleach catalyst and/or transition metal bleach catalyst), from 0% to 0.1%;
Other bleach (such as reducing bleach and/or pre-formed peracid), from 0% to 10%;
Chelant (such as ethylenediamine-N′N′-disuccinic acid (EDDS) and/or hydroxyethane diphosphonic acid (HEDP), from 0.2% to 1%;
Photobleach (such as zinc and/or aluminium sulphonated phthalocyanine), from 0% to 0.1%;
Hueing agent (such as direct violet 99, acid red 52, acid blue 80, direct violet 9, solvent violet 13 and any combination thereof), from 0% to 1%;
Brightener (such as brightener 15 and/or brightener 49), from 0.1% to 0.4%;
Fabric softener (such as montmorillonite clay and/or polydimethylsiloxane (PDMS)), from 0% to 4%;
Flocculant (such as polyethylene oxide), from 0% to 1%;
Suds suppressor (such as silicone and/or fatty acid), from 0% to 0.1%;
Perfume (such as perfume microcapsule, spray-on perfume, starch encapsulated perfume accords, perfume loaded zeolite, and any combination thereof), from 0.1% to 1%; and
Aesthetics (such as colored soap rings and/or colored speckles/noodles), from 0% to 1%; and optionally a protease (such as Savinase, Coronase, Ovozyme, Kannase, Liquanase, Polarzyme, Purafect, Purafast, Properase, Excellase, FN3, FN4, Effectenz P, Preferenz P, Progress Uno, Progress Excel, Blaze, Excellenz P), from about 0.05 wt % to about 0.2 wt %; Optionally an additional amylase differing from the amylase variant described herein (such as Termamyl(R), Termamyl Ultra (R), Natalase (R), Optisize HT Plus (R), Purastar, Powerase (R), Stainzyme (R), Preferenz S, Effectenz S, Amplify, Amplify Prime, Achieve alpha, Excellenz S and any combination thereof), from about 0.05 wt % to about 0.2 wt %;
Optionally Cellulase (such as Carezyme, Celluclean, Puradax, Biotouch, Whitezyme, Revitalenz, and combinations thereof), from 0.05% to 0.2%;
Optionally Lipase (such as Lipex, Lipolex, Lipoclean, Preferenz L, and any combination thereof), from 0.05% to 0.2%;
Optionally other enzyme (such as xyloglucanase, cutinase, pectate lyase (such as Xpect), Mannanase (such as Mannanway, Mannastar, Marvellenz, Effectenz M, Preferenz M, Preferenz F, and combinations thereof) bleaching enzyme, and combinations thereof), from 0.05% to 0.2%;
In another embodiment, the amylase variant described herein is included in a detergent composition comprising one or more, preferably all, compounds selected from the group consisting of (all percentages are w/w):

A formulation comprising the amylase variant described herein, from 0.05% to 1.0%;
Carboxyl group-containing polymer (comprising from about 60% to about 70% by mass of an acrylic acid-based monomer (A); and from about 30% to about 40% by mass of a sulfonic acid group-containing monomer (B); and wherein the average molecular weight is from about 23,000 to about 50,000 preferably in the range of from about 25,000 to about 38,000 as described in WO2014032269), from about 0.5 wt % to ab out 1.5 wt %;
Anionic detersive surfactant (such as alkyl benzene sulphonate, alkyl ethoxylated sulphate and mixtures thereof), from about 8 wt % to about 15 wt %;
Non-ionic detersive surfactant (such as alkyl ethoxylated alcohol) from about 0.5 wt % to 4 wt %;
Cationic detersive surfactant (such as quaternary ammonium compounds), from about 0 wt % to about 4 wt %;
Other detersive surfactant (such as zwiterionic detersive surfactants, amphoteric surfactants and mixtures thereof), from about 0 wt % to 4 wt %;
Carboxylate polymer (such as co-polymers of maleic acid and acrylic acid) from about 1 wt % to about 4 wt %;
Polyethylene glycol polymer (such as a polyethylene glycol polymer comprising poly vinyl acetate side chains), from about 0 wt % to about 4 wt %;
Polyester soil release polymer (such as Repel-O-Tex (R) and/or Texcare (R) polymers), from about 0.1 wt % to about 2 wt %;
Cellulosic polymer (such as carboxymethyl cellulose, methyl cellulose and combinations thereof) from about 0.5 wt % to about 2 wt %;
Other polymer (such as amine polymers, dye transfer inhibitor polymers, hexamethylenediamine derivative polymers, and mixtures thereof), from about 0 wt % to about 4 wt %;
Zeolite builder and phosphate builder (such as zeolite 4A and/or sodium tripolyphosphate), from about 0 wt % to about 4 wt %;
Other builder (such as sodium citrate and/or citric acid), from about 0 wt % to about 3 wt %;
Carbonate salt (such as sodium carbonate and/or sodium bicarbonate), from about 15 t % to about 30 wt %;
Silicate salt (such as sodium silicate), from about 0 wt % to about 10 wt %; Filler (such as sodium sulphate and/or bio-fillers), from about 10 wt % to about 40 wt %;
Source of available oxygen (such as sodium percarbonate), from about 10 wt % to about 20 wt %;
Bleach activator (such as tetraacetylethylene diamine (TAED) and/or nonanoyloxybenzenesulphonate (NOBS), from about 2 wt % to about 8 wt % 2;
Bleach catalyst (such as oxaziridinium-based bleach catalyst and/or transition metal bleach catalyst), from about 0 wt % to about 0.1 wt %;
Other bleach (such as reducing bleach and/or pre-formed peracid), from about 0 wt % to about 10 wt %;
Chelant (such as ethylenediamine-N′N′-disuccinic acid (EDDS) and/or hydroxyethane diphosphonic acid (HEDP), from about 0.2 wt % to about 1 wt %;
Photobleach (such as zinc and/or aluminium sulphonated phthalocyanine), from about 0 wt % to about 0.1 wt %;
Hueing agent (such as direct violet 99, acid red 52, acid blue 80, direct violet 9, solvent violet 13 and any combination thereof), from about 0 wt % to about 0.5 wt %;
Brightener (such as brightener 15 and/or brightener 49), from about 0.1 wt % to about 0.4 wt %; Fabric softener (such as montmorillonite clay and/or polydimethylsiloxane (PDMS)), from 0 wt % to 15 wt %;
Flocculant (such as polyethylene oxide), from 0 wt % to 1 wt %;
Suds suppressor (such as silicone and/or fatty acid), from 0 wt % to 0.1 wt %;
Perfume (such as perfume microcapsule, spray-on perfume, starch encapsulated perfume accords, perfume loaded zeolite, and any combination thereof), from 0.1 wt % to 1 wt %; and Aesthetics (such as colored soap rings and/or colored speckles/noodles), from 0 wt % to 1 wt %, Optionally a protease (such as Savinase, Coronase, Ovozyme, Kannase, Liquanase, Polarzyme, Purafect, Purafast, Properase, Excellase, FN3, FN4, Effectenz P, Preferenz P, Progress Uno, Progress Excel, Blaze, Excellenz P), from about 0.05 wt % to about 0.2 wt %, Optionally additional amylase differing from the amylase variant described herein (such as Termamyl(R), Termamyl Ultra (R), Natalase (R), Optisize HT Plus (R), Purastar, Powerase (R), Stainzyme (R), Preferenz S, Effectenz S, Amplify, Amplify Prime, Achieve alpha, Excellenz S), from about 0.05 wt % to about 0.2 wt %,
Optionally, Cellulase (such as Carezyme (R), Celluzyme (R), Puradax, Celluclean (R), Biotouch, Whitezyme, Revitalenz, and combinations thereof, typically having an enzyme activity of about from 10 to 50 mg active enzyme/g), from about 0.05 wt % to 0.5 wt %
Optionally, Lipase (such as Lipex (R), Lipolex (R), Lipoclean (R), Preferenz L, and any combination thereof, typically having an enzyme activity of from about 10 mg to about 50 mg active enzyme/g), from about 0.2 wt % to about 1 wt %
Optionally, other enzyme (such as xyloglucanase (e.g., Whitezyme (R)), cutinase, pectate lyase (e.g., Xpect), mannanase, (e.g., Mannanway, Mannastar, Marvellenz, Effectenz M, Preferenz M, Preferenz F, and combinations thereof), bleaching enzyme, typically having an enzyme activity of from about 10 mg to about 50 mg active enzyme/g), from 0 wt % to 2 wt %,

Further preferred detergent composition comprise the components listed below (all percentages are w/w):
Aqua, Alcohol Ethoxy Sulfate, Alcohol Ethoxylate, Amino Oxide, Citrid Acid, C₁₂-18 topped palm kernel fatty acid, Ethanol, 1,2 Propanediol, Sodium Formate, Calcium Chloride, Sodium hydroxide, Silicone Emulsion, Trans-sulphated EHDQ, an amylase variant as described herein;
Linear sodium alkyl benzene sulfonate 8,8%, Ethoxylated fatty alcohol C₁₂-18 (7 EO) 4,7%, Sodium soap 3,2%, Antifoam DC2-4248S 3,9%, Sodium aluminium silicate zeolite 4A 28,3%, Sodium carbonate11,6%, Sodium salt of a copolymer from acrylic and maleic acid (Sokalan CP5) 2,4%, Sodium silicate 3,0%, Carboxymethylcellulose 1,2%, Dequest 2066 2,8%, Optical whitener 0.2%, Sodium sulfate 6,5%, 0.4% of a formulation comprising the amylase variant as described herein;
12% LAS, 11% AEO Biosoft N25-7 (NI), 7% AEOS (SLES), 6% MPG (monopropylene glycol), 3% ethanol, 3% TEA, 2.75% cocoa soap, 2.75% soya soap, 2% glycerol, 2% sodium hydroxide, 2% sodium citrate, 1% sodium formate, 0.2% DTM PA and 0.2% PCA, an amylase variant as described herein;
5-15% Anionic surfactants; <5% Non-ionic surfactants, Phosphonates, Soap; Enzymes, Optical brighteners, Benzisothiazolinone, Methylisothiazolinone, Perfumes, Alpha-isomethyl ionone, Citronellol, Geraniol, Linalool, an amylase variant as described herein;
Aqua, Sodium Dodecylbenzenesulfonate, C14-C15 Pareth-7, Sodium Citrate, Propylene Glycol, Sodium Palm Kernelate, Sodium Laureth Sulfate, MEA Dodecylbenzenesulfonage, Sulfated Ethoxylated Hexamethylenediamine Quaternized, Sodium Cumenesulfonate, Perfume, Co-polymer of PEG/Vinyl Acetate, Sodium formate, Hydrogenated Castor Oil, Sodium Diethylenetri-amine Pentamethylene Phosphonate, PEG/PPG-10/2 Propylheptyl Ether, Butyophenyl Methylpropional, Polyvinylpyridine-N-Oxide, Sorbitol, Glycerin, Ethanolamine, Sodium Hydroxide, Alpha-Isomethyllonone, Calcium Chloride, Geraniol, Linalool, Citronelllol, Tripropylene Glycol, Benzisothiazolinone, Dimethicone, Sodium Acetate, Cellulase, Colorant, Glyceryl Stearate, Hydroxyethylcellulose, Silica, an amylase variant as described herein;
Aqua, Sodium Laureth Sulfate, Propylene Glycol, C₁₄-C₁₅ Pareth-7, Sodium citrate, Sodium Palm Kernelate, Alcohol, Sodium Formate, Sulfated Ethoxylated Hexamethylenediamine Quaternized, Sodium Hydroxide, Perfume, Polyvinylpyridine-N-Oxide, Sorbitol, Calcium Chloride, Glycerin, Sodium Acetate, Colorant, Cellulase, an amylase variant as described herein;
Aqua, Sodium Laureth Sulfate, Propylene Glycol, C14-C15 Pareth-7, Sodium citrate, Sodium Palm Kernelate, Alcohol, Sodium Formate, Sulfated Ethoxylated Hexamethylenediamine Quaternized, Sodium Hydroxide, Perfume, Sorbitol, Calcium Chloride, Glycerin, Sodium Acetate, Colorant, Cellulase, an amylase variant as described herein;
Aqua, Sodium Laureth Sulfate, Propylene Glycol, C14-C15 Pareth-7, Sodium citrate, Sodium Palm Kernelate, Alcohol, Sodium Formate, Sulfated Ethoxylated Hexamethylenediamine Quaternized, Sodium Hydroxide, Sorbitol, Calcium Chloride, Glycerin, Sodium Acetate, Cellulase, Silica, an amylase variant as described herein;
Aqua, Sodium Dodecylbenzenesulfonate, C14-C15 Pareth-7, Sodium Citrate, Propylene Glycol, Sodium Palm Kernelate, Sodium Laureth Sulfate, MEA Dodecylbenzenesulfonage, Sulfated Ethoxylated Hexamethylenediamine Quaternized, Sodium Cumenesulfonate, Perfume, Co-polymer of PEG/Vinyl Acetate, Sodium formate, C12-C14 Pareth-7, Hydrogenated Castor Oil, Sodium Diethylenetriamine Pentamethylene Phosphonate, PEG/PPG-10/2 Propylheptyl Ether, Butyophenyl Methylpropional, Fluorescent Brightener, Sorbitol, Glycerin, Ethanolamine, Sodium Hydroxide, Alpha-Isomethyl lonone, Calcium Chloride, Geraniol, Linalool, Citronelllol, Tripropylene Glycol, Sodium Chloride, Benzisothiazolinone, Dimethicone, Sodium Acetate, Cellulase, Colorant, Glyceryl Stearate, Hydroxyethylcellulose, Silica, an amylase variant as described herein;
15-30% Anionic surfactants, Non-ionic surfacts, 5-15% Soap, <5% Polycarboxylates, Perfume, Phosphates, Optical Brighteners, an amylase variant as described herein;
15-30% Anionic Surfactants,5-15% Non-Ionic Surfactants, Soap, Benzisothiazolinone, Methylisothiazolinone, Perfumes, an amylase variant as described herein;
11% LAS, 2% AS/AEOS, 2% soap, 3% AEO, 15.15% sodium carbonate, 3% sodium slilcate, 18.75% zeolite, 0.15% chelant, 2% sodium citrate, 1.65% AA/MA copolymer, 2.5% CMC and 0.5% SRP, an amylase variant as described herein;
16.5% LAS, 15% zeolite, 12% sodium disilicate, 20% sodium carbonate, 1% sokalan, 35.5% sodium sulfate, an amylase variant as described herein;
15-30% Anionic surfactants, <5% Nonionic surfactants, Phosphonates, Polycarboxylates, Zeo-lites; Enzymes, Perfumes, Hexyl cinnamal, an amylase variant as described herein;
15-30% of the following: anionic surfactants, oxygen-based bleaching agent and zeolites, less than 5% of the following: non-ionic surfactants, phosphonates, polycarboxylates, soap,

Further ingredients: Perfumes, Hexyl cinnamal, Benzyl salicylate, Linalool, optical brighteners, Enzymes and Citronellol, an amylase variant as described herein;
Water, Alcohol Ethoxysulfate, Diethylene Glycol, Alcohol Ethoxylate, Ethanolamine, Linear Alkyl Benzene Sulfonate, Sodium Fatty Acids, Polyethyleneimine Ethoxylate, Citric Acid, Sodium Cumene Sulfonate, Propylene Glycol, DTPA, Disodium Diaminostilbene Disulfonate, Dipro-pylethyl Tetramine, Sodium Hydroxide, Sodium Formate, Calcium Formate, Dimethicone, Liqui-tint™, Hydrogenated Castor Oil, Fragrance, an amylase variant as described herein;
Linear alkylbenzene sulfonate, propylene glycol, citric acid, sodium hydroxide, ethanolamine, ethanol, alcohol sulfate, polyethyleneimine ethoxylate, sodium fatty acids, diquaternium ethoxy-sulfate, diethylene glycol, laureth-9, alkyldimethylamine oxide, fragrance, disodium diaminostilbene disulfonate, DTPA, sodium formate, calcium formate, polyethylene glycol 4000, mannanase, Liquitint™ Blue, dimethicone, an amylase variant as described herein;
Water, sodium alcoholethoxy sulfate, propylene glycol, ethanol, linear alkylbenzene sulfonate sodium, salt, polyethyleneimine ethoxylate, diethylene glycol, trans sulfated & ethoxylated hexa-methylene diamine, alcohol ethoxylate, linear alkylbenzene sulfonate, MEA salt, sodium for-mate, sodium alkyl sulfate, DTPA, amine oxide, calcium formate, disodium diaminostilbene, di-sulfonate, dimethicone, benzisothiazolinone, an amylase variant as described herein;
Water, alcoholethoxy sulfate, linear alkylbenzene sulfonate, diethylene glycol, propylene glycol, ethanolamine, citric acid, alcohol sulfate, sodium hydroxide, polyethyleneimine, ethoxylate, sodium fatty acids, ethanol, Laureth-9, diquaternium ethoxysulfate, lauramine oxide, sodium cumene, sulfonate, fragrance, DTPA, disodium, diaminostilbene, disulfonate, sodium formate, disodium distyrylbiphenyl, disulfonate, calcium formate, polyethylene glycol 4000, mannanase, pectinase, Liquitint™ Blue, dimethicone, an amylase variant as described herein;
Water, alcoholethoxy sulfate, propylene glycol, sodium fatty acids, laurtrimonium chloride, ethanol, sodium hydroxide, sodium cumene sulfonate, citric acid, ethanolamine, diethylene glycol, silicone polyether, fragrance, polyethylene-imine ethoxylate, amylase, Laureth-9, DTPA, poly-acrylamide quaternium chloride, disodium diaminostilbene disulfonate, sodium formate, Liqui-tint™ Orange, dipropylethyl tetraamine, dimethicone, cellulase, an amylase variant as described herein;
Water, sodium alcoholethoxy sulfate, sodium alkyl sulfate, MEA citrate, linear alkylbenzene sulfonate, MEA salt, propylene glycol, diethylene glycol, polyethyleneimine ethoxylate, ethanol, sodium fatty acids, ethanolamine, lauramine oxide, Laureth-9, DTPA, sodium cumene sulfonate, sodium formate, calcium formate, linear alkylbenzene sulfonate, sodium salt, alcohol sulfate, sodium hydroxide, diquaternium ethoxysulfate, fragrance, amylase, mannanase, pectinase, disodium diaminostilbene disulfonate, benzisothiazolinone, Liquitint™ Blue, dimethicone, dipropylethyl tetraamine, an amylase variant as described herein;
Water, Sodium alcoholethoxy sulfate, MEA citrate, Sodium Alkyl Sulfate, alcohol ethoxylate, linear alkylbenzene sulfonate, MEA salt, sodium fatty acids, polyethyleneimine ethoxylate, dieth-ylene glycol, propylene glycol, diquaternium ethoxysulfate, polyethyleneimine, ethoxylate propoxylate, ethanol, sodium cumene sulfonate, fragrance, DTPA, disodium diaminostilbene di-sulfonate, Mannanase, cellulase, sodium formate, calcium formate, Lauramine oxide, Liquitint™ Blue, Dimethicone/polydimethyl silicone, an amylase variant as described herein;
Water, alcoholethoxy sulfate, linear alkylbenzene sulfonate, alcohol ethoxylate, citric acid, Eth-anolamine, sodium fatty acids, diethylene glycol, propylene glycol, sodium hydroxide, polyethyleneimine ethoxylate, silicone polyether, ethanol, amylase, sodium cumene sulfonate, diquaternium ethoxysulfate, Laureth-9, fragrance, DTPA, disodium diaminostilbene disulfonate, disodium distyrylbiphenyl disulfonate, sodium formate, calcium formate, mannanase, Liquitint™ Orange, dimethicone, polyacrylamide quaternium chloride, cellulase, dipropylethyl tetraamine, an amylase variant as described herein;
Water, alcoholethoxy sulfate, diethylene glycol, monoethanolamine citrate, sodium formate, propylene glycol, linear alkylbenzene sulfonates, ethanolamine, ethanol, poly-ethyleneimine ethoxylate, benzisothiazolin, calcium formate, citric acid, diethylenetriamine pentaacetate sodium, dimethicone, diquaternium ethoxysulfate, disodium diaminostilbene disulfonate, Laureth-9, mannanase, sodium cumene sulfonate, sodium fatty acids, an amylase variant as described herein;
Water, alcoholethoxy sulfate, MEA Citrate, alcohol sulfate, Alcohol ethoxylate, Linear alkylbenzene sulfonate MEA, sodium fatty acids, polyethyleneimine ethoxylate, diethylene glycol, propylene glycol, diquaternium ethoxysulfate, polyethyleneimine ethoxylate propoxylate, ethanol, sodium cumene sulfonate, fragrance, DTPA, disodium diaminostilbene disulfonate, mannanase, cellulase, sodium formate, calcium formate, lauramine oxide, Liquitint™ Blue, dimethicone, an amylase variant as described herein;
Water, sodium alcoholethoxy sulfate, MEA Citrate, Linear alkylbenzene sulfonate: sodium salt, Alcohol ethoxylate, Linear alkylbenzene sulfonate: MEA salt, sodium fatty acids, polyethyleneimine ethoxylate, diethylene glycol, propylene glycol, di-quaternium ethoxysulfate, polyethyleneimine ethoxylate propoxylate, ethanol, sodium cumene sulfonate, citric acid, DTPA, disodium diaminostilbene disulfonate, sodium formate, calcium for-mate, dimethicone, an amylase variant as described herein;
Water, alcohol ethoxylate sulfate, linear alkylbenzene sulfonate Sodium/Mea salts, propylene glycol, diethylene glycol, sodium formate, ethanol, sodium fatty acids, fragrance, lauramine ox-ide, DTPA, Polyethylene amine ethoxylate, calcium formate, disodium diaminostilbene disulfonate, dimethicone, tetramine, Liquitint™ Blue, an amylase variant as described herein;
Linear alkylbenzene sulfonates, C12-16 Pareth-9, propylene glycol, alcoholethoxy sulfate, water, polyethyleneimine ethoxylate, glycerine, fatty acid salts, PEG-136 polyvinyl acetate, ethylene Diamine disuccinic salt, monoethanolamine citrate, sodium bisulfite, diethylenetriamine pentaacetate sodium, disodium distyrylbiphenyl disulfonate, calcium formate, mannanase, exyloglucanase, sodium formate, hydrogenated castor oil, dyes, subtilisin, benzisothiazolin, perfume, an amylase variant as described herein;
Deionized water, Dipropylene Glycol Butyl Ether, Sodium Alkyl Sulfate, Hydrogen Peroxide, Ethanol, Magnesium Sulfate, Alkyl Dimethyl Amine Oxide, Citric Acid, Sodium Hydroxide, Tri-methoxy Benzoic Acid, Fragrance, an amylase variant as described herein;
Water, Alkyl Ethoxylate, Linear Alkylbenzenesulfonate, Hydrogen Peroxide, Diquaternium Ethoxysulfate, Ethanolamine, Disodium Distyrylbiphenyl Disulfonate, tetrabutyl Ethylidinebisphenol, F&DC Yellow 3, Fragrance, an amylase variant as described herein;
Sodium percarbonate, sodium sulfate, sodium carbonate, sodium aluminosilicate, nonanoyloxy benzene sulfonate, sodium polyacrylate, water, sodium alkylbenzenesulfonate, DTPA, polyethylene glycol, sodium palmitate, amylase, modified starch, FD&C Blue 1, fragrance, an amylase variant as described herein;
Water, Alkyl Ethoxylate, MEA Borate, Linear Alkylbenzenesulfonate, Propylene Glycol, Diquaternium Ethoxysulfate, Calcium Chlorideenzyme, Ethanolamine, Benzoisothiazolinone, Sodium Citrate, Sodium Hydroxide, Fragrance, an amylase variant as described herein;
Water, Alkyl Amine Oxide, Dipropylene Glycol Phenyl Ether, Hydrogen Peroxide, Citric Acid, Ethylene Diamine Disuccinic Acid Sodium salt, Sodium Alkyl Sulfate, Fragrance, an amylase variant as described herein;
Sodium bicarbonate, sodium carbonate, sodium percarbonate, alcohol ethoxylate, sodium chloride, maleic/acrylic copolymer, nonanoyloxy benzene sulfonate, sodium sulfate, colorant, diethylenetriamine pentaacetate sodium salt, hydrated aluminosilicate (zeolite), polyethylene glycol, sodium alkylbenzene sulfonate, sodium palmitate, starch, water, fragrance, an amylase variant as described herein;
Polyvinyl Alcoholpouch film, wherein there is packed a liquid part and a powder part: Liquid Ingredients: Dipropylene Glycol, diquaternium Ethoxysulfate, Water, Glycerin, Liquitint™ Orange, an amylase variant as described herein;
power ingredients: sodium percarbonate, nonanoyloxy benzene sulfonate, sodium carbonate, sodium sulfate, sodium aluminosilicate, sodium polyacrylate, sodium alkylbenzenesulfonate, maleic/acrylic copolymer, water, polyethylene glycol, sodium palmitate, modified starch, glycerine, DTPA, fragrance, an amylase variant as described herein;
Water, sodium alcoholethoxy sulfate, linear alkyl benzene sulfonate, sodium/MEA salts, MEA citrate, propylene glycol, polyethyleneimine ethoxylate, ethanol, diethylene glycol, polyethyleneimine propoxyethoxylate, sodium fatty acids, sodium cumene sulfonate, DTPA, fragrance, disodium diaminostilbene disulfonate, calcium formate, sodium formate, gluconase, dimethicone, Liquitint™ Blue, mannanase, an amylase variant as described herein;
Sodium Carbonate, Sodium Aluminosilicate, Sodium Sulfate, Linear Alkylbenzene Sulfonate, Bentonite, Water, Sodium Percarbonate, Sodium Polyacrylate, Silicate, Alkyl Sulfate, Nonanoyloxybenzenesulfonate, DTPA, Polyethylene Glycol 4000, Silicone, Ethoxylate, fragrance, Polyethylene Oxide, Palmitic Acid, Disodium Diaminostilbene Disulfonate, Liquitint™ Red, FD&C Blue 1, Cellulase, an amylase variant as described herein;
Water, sodium alcoholethoxy sulfate, MEA citrate, linear alkyl benzene sulfonate: sodium/MEA salts, propylene glycol, polyethyleneimine ethoxylate, ethanol, diethylene glycol, polyethylene-imine, propoxyethoxylate, diquaternium ethoxysulfate, alcohol sulfate, dimethicone, fragrance, sodium fatty acids, DTPA, sodium bisulfite, disodium diaminostilbene disulfonate, gluconase, castor oil, calcium formate, MEA, styrene acrylate copolymer, sodium formate, Liquitint™ Blue, an amylase variant as described herein;
Water, sodium alcoholethoxy sulfate, MEA citrate, linear alkyl benzene sulfonate: sodium/MEA salts, propylene glycol, ethanol, diethylene glycol, polyethyleneimine propoxyethoxylate, polyethyleneimine ethoxylate, alcohol sulfate, dimethicone, fragrance, sodium fatty acids, DTPA, sodium bisulfite, disodium diaminostilbene disulfonate, castor oil, calcium formate, MEA, styrene acrylate copolymer, propanaminium propanamide, gluconase, sodium formate, Liquitint™ Blue, an amylase variant as described herein;
Water, sodium alcoholethoxy sulfate, MEA citrate, linear alkyl benzene sulfonate: sodium/MEA salts, propylene glycol, polyethyleneimine ethoxylate, ethanol, diethylene glycol, polyethyleneimin propoxyethoxylate, diquaternium ethoxysulfate, alcohol sulfate, dimethicone, fragrance, sodium fatty acids, DTPA, sodium bisulfite, disodium diaminostilbene disulfonate, gluconase, castor oil, calcium formate, MEA, styrene acrylate copolymer, propanaminium propanamide, sodium formate, Liquitint™ Blue, an amylase variant as described herein;
Sodium Carbonate, Sodium Aluminosilicate, Alkyl Sulfate, Sodium Sulfate, Linear Alkylbenzene Sulfonate, Water, Sodium polyacrylate, Silicate, Ethoxylate, Sodium percarbonate, Polyethylene Glycol 4000, Disodium Diaminostilbene Disulfonate, Silicone, Cellulase, an amylase variant as described herein;
Sodium Carbonate, Sodium Aluminosilicate, Sodium Sulfate, Linear Alkylbenzene Sulfonate, Alkyl Sulfate, Sodium Percarbonate, Water, Sodium Polyacrylate, Silicate, Nonanoyloxybenzenesulfonate, Ethoxylate, Polyethylene Glycol 4000, Fragrance, DTPA, Disodium Diaminostilbene Disulfonate, Palmitic Acid, Silicone, Cellulase, an amylase variant as described herein;
Sodium Carbonate, Sodium Aluminosilicate, Sodium Sulfate, Linear Alkylbenzene Sulfonate, Water, Nonanoyloxybenzenesulfonate, Alkyl Sulfate, Sodium Polyacrylate, Silicate, Sodium Percarbonate, Ethoxylate, Polyethylene Glycol 4000, Fragrance, DTPA, Palmitic Acid, Disodium Diaminostilbene Disulfonate, Silicone, Cellulase, an amylase variant as described herein;
Sodium Carbonate, Sodium Aluminosilicate, Sodium Sulfate, Sodium Percarbonate, Alkyl Sulfate, Linear Alkylbenzene Sulfonate, Water, Nonanoyloxybenzenesulfonate, Sodium Polyacrylate, Silicate, Ethoxylate, Polyethylene Glycol 4000, DTPA, Fragrance, Palmitic Acid, Disodium, Diaminostilbene Disulfonate, FD&C Blue 1, Silicone, Cellulase, Alkyl Ether Sulfate, an amylase variant as described herein;
Sodium Carbonate, Sodium Aluminosilicate, Sodium Sulfate, Linear Alkylbenzene Sulfonate, Sodium Percarbonate, Nonanoyloxybenzenesulfonate, Alkyl Sulfate, Water, Silicate, Sodium Polyacrylate, Ethoxylate, Polyethylene Glycol 4000, Fragrance, DTPA, Palmitic Acid, Disodium Diaminostilbene Disulfonate, Silicone, FD&C Blue 1, Cellulase, Alkyl Ether Sulfate, an amylase variant as described herein;
Sodium Carbonate, Sodium Aluminosilicate, Sodium Sulfate, Linear Alkylbenzene Sulfonate, Sodium Percarbonate, Alkyl Sulfate, Water, Sodium Polyacrylate, Silicate, Nonanoyloxybenzenesulfonate, Ethoxylate, Polyethylene Glycol 4000, DTPA, Fragrance, Cellulase, Disodium Diaminostilbene Disulfonate, Silicone, FD&C Blue 1, an amylase variant as described herein;
Water, Sodium alcoholethoxy sulfate, MEA citrate, linear alkylbenzene sulfonate, sodium salt, linear alkylbenzene sulfonate: MEA salt, alcohol ethoxylate, sodium fatty acids, propylene glycol, diethylene glycol, polyethyleneimine ethoxylate propoxylate, diquaternium ethoxysulfate, Ethanol, sodium cumene sulfonate, fragrance, DTPA, Sodium bisulfate, disodium diaminostilbene disulfonate, Mannanase, cellulase, sodium formate, calcium formate, Lauramine oxide, Liquitint™ Blue, Dimethicone/polydimethyl silicone, an amylase variant as described herein;
Water, sodium alcoholethoxy sulfate, linear alkyl benzene sulfonate: sodium/MEA salts, MEA citrate, propylene glycol, polyethyleneimine ethoxylate, fragrance, ethanol, diethylene glycol, polyethyleneimine propoxyethoxylate, alcohol sulfate, sodium fatty acids, DTPA, disodium diaminostilbene disulfonate, MEA, mannanase, gluconase, sodium formate, dimethicone, Liquitint™ Blue, tetramine, an amylase variant as described herein;
Water, Sodium alco-holethoxy sulfate, MEA citrate, linear alkylbenzene sulfonate, sodium salt, linear alkylbenzene sulfonate: MEA salt, alcohol ethoxylate, sodium fatty acids, propylene glycol, diethylene glycol, polyethyleneimine ethoxylate propoxylate, diquaternium ethoxysulfate, ethanol, sodium cumene sulfonate, fragrance, DTPA, Sodium bisulfate, disodium diaminostilbene disulfonate, Mannanase, cellulase, sodium formate, calcium formate, Lauramine oxide, Liquitint™ Blue, Dimethicone/polydimethyl silicone, an amylase variant as described herein;
Sodium Carbonate, Sodium Aluminosilicate, Sodium Sulfate, Linear Alkylbenzene Sulfonate, Sodium Percarbonate, Nonanoyloxybenzenesulfonate, Alkyl Sulfate, Water, Silicate, Sodium Polyacrylate Ethoxylate, Polyethylene Glycol 4000, Fragrance, DTPA, Palmitic Acid, Disodium Diaminostilbene Disulfonate, Silicone, FD&C Blue 1, Cellulase, Alkyl Ether Sulfate, an amylase variant as described herein; or
Aqua, dodecylbenzenesulfonsäure, laureth-11, peg-75 lanolin, propylene glycol, alcohol de-nat., potassium soyate, potassium hydroxide, disodium cocoamphodiacetate, ethylendiamine triacetate cocosalkyl acetamide, parfum, zinc ricinoleate, sodium chloride, benzisothiazolinone, methylisothiazolinone, ci 16255, benzyl alcohol, an amylase variant as described herein.

Preferably, the compositions listed above including a protease further comprise 4-FPBA and/or a peptide aldehyde protease inhibitor, most preferably Z-GAY or Z-VAL.

The amylase variant described herein can be comprised in one of the following detergent compositions.

| | | wt % in |
|---|---|---|
| ES1-C | | formulation |
| Maranil DBS/LC | linear alkylbenzene sulfonate, | 5.5 |
| | anionic surfactant | |
| Edenor coco fatty acid | C₁₂-C₁₈ coco fatty acid | 2.4 |
| Lutensol AO7 | alkyl polyethyleneglycol ether, | 5.5 |
| | non-ionic surfactant | |
| Texapon N70 | sodium laureth sulfate 2EO, | 5.5 |
| | anionic surfactant | |
| 1,2 propylene glycol | | 6.0 |
| C₂H₅OH | | 2.0 |
| NaOH | | 2.2 |
| Citrate | | 3.0 |
| a formulation comprising | | 0.05-1.0 |
| the amylase variant | | |
| described herein and | | |
| optionally one or more | | |
| enzymes, preferably a | | |
| protease | | |
| pH | | 8-8.5 |
| Water | | up to 100% |

| | % wt in formulation |
|---|---|
| Linear C10-C13-alkyl benzene sulfonic acid | 10.0 |
| C12-C14-fatty alcohol ether sulfate (2 EO) | 10.0 |
| C12-C14-fatty alcohol ethoxylate (7EO) | 20.0 |
| C12-C18 coco fatty acid | 5.0 |
| Na-Citrat | 1.5 |
| NaOH 50% | |
| Propylenglykol | 10.0 |
| a formulation comprising the amylase variant | 0.05-1.0 |
| described herein and optionally one or more | |
| enzymes, preferably a protease | |
| water | Ad 100% |

| | % wt in formulation |
|---|---|
| Linear C10-C13-alkyl benzene sulfonic acid | 10.0 |
| C12-C14-fatty alcohol ether sulfate (2 EO) | 10.0 |
| C12-C14-fatty alcohol ethoxylate (7EO) | 20.0 |
| C12-C18 coco fatty acid | 5.0 |
| MGDA | 1.5 |
| NaOH 50% | |
| Propylenglykol | 10.0 |
| a formulation comprising the amylase variant | 0.05-1.0 |
| described herein and optionally one or more | |
| enzymes, preferably a protease | |
| Water | Ad 100% |

| | % wt in formulation |
|---|---|
| Linear C10-C13-alkyl benzene sulfonic acid | 10.0 |
| C12-C14-fatty alcohol ether sulfate (2 EO) | 10.0 |
| C12-C14-fatty alcohol ethoxylate (7EO) | 20.0 |
| C12-C18 coco fatty acid | 5.0 |
| GLDA | 1.5 |
| NaOH 50% | |
| Propylenglykol | 10.0 |
| a formulation comprising the amylase variant | 0.05-1.0 |
| described herein and optionally one or more | |
| enzymes, preferably a protease | |
| Water | Ad 100% |

| | % wt in formulation |
|---|---|
| Linear C10-C13-alkyl benzene sulfonic acid | 10.0 |
| C12-C14-fatty alcohol ether sulfate (2 EO) | 10.0 |
| C12-C14-fatty alcohol ethoxylate (7EO) | 20.0 |
| C12-C18 coco fatty acid | 5.0 |
| Phosphonate (e.g., HEDP or DTPMP) | 1.5 |
| NaOH 50% | |
| Propylenglykol | 10.0 |
| a formulation comprising the amylase variant | 0.05-1.0 |
| described herein and optionally one or more | |
| enzymes, preferably a protease | |
| Water | Ad 100% |

% wt in
formulation

| | | | | | | | | | | | | |
|---|---|---|---|---|---|---|---|---|---|---|---|---|
| Alcohol | 0.6 | 0 | 1 | 0 | 0 | 5.2 | 1.2 | 5 | 4 | 0.5 | 0.5 | 0 |
| ethoxylate 7EO | | | | | | | | | | | | |
| Coco fatty | 1.2 | 0 | 0 | 0 | 0 | 0 | 0 | 0 | 0 | 0.3 | 0 | 0.6 |
| acid C12-18 | | | | | | | | | | | | |
| C12-C14-fatty | 1.5 | 0 | 0 | 0 | 0 | 6 | 0 | 3.9 | 4.4 | 1.6 | 0 | 0 |
| alcohol ether | | | | | | | | | | | | |
| sulfate (2 EO) | | | | | | | | | | | | |
| Linear alkyl | 12.1 | 11.2 | 13.6 | 21.9 | 18.7 | 12.7 | 7.6 | 12.1 | 11.5 | 12.2 | 6.5 | 10.4 |
| benzene | | | | | | | | | | | | |
| sulphonic | | | | | | | | | | | | |
| acid | | | | | | | | | | | | |
| Bleach | 0 | 0 | 0 | 0 | 0 | 0 | 0.2 | 9.5 | 9.5 | 0.5 | 0.8 | 2.2 |
| activator | | | | | | | | | | | | |
| Percarbonate | 0 | 0 | 0 | 0 | 0 | 0 | 3.6 | 19.4 | 16.6 | 2.2 | 11.5 | 5.8 |
| AcetateNa | 0 | 0 | 0 | 0.1 | 0 | 0.1 | 0 | 6.7 | 7.1 | 0.3 | 1 | 0.7 |
| CitrateNa | 0 | 0 | 0 | 0 | 0 | 14 | 0 | 1.6 | 8.2 | 0.3 | 0.9 | 1.7 |
| Na Silicate | 27.9 | 5.8 | 6.6 | 2 | 15 | 20.3 | 3.6 | 11.3 | 16.4 | 10.2 | 9.1 | 16.5 |
| Na Carbonate | 17.2 | 35 | 37.3 | 30.1 | 37 | 1 | 21.6 | 8.7 | 1.4 | 8 | 22.9 | 14.8 |
| Na Phospahte | 0 | 0 | 0 | 14 | 0.3 | 0 | 0 | 0 | 0 | 0 | 0 | 0 |
| Na Hydrogen- | 0.7 | 0.9 | 0.5 | 2.7 | 0.4 | 10.5 | 0.2 | 2.8 | 1.6 | 0.8 | 0.5 | 0.5 |
| carbonate | | | | | | | | | | | | |
| Zeolite4A | 4.2 | 0.1 | 5.1 | 10.2 | 1.8 | 11.6 | 1.6 | 1.4 | 2.4 | 1.6 | 1.8 | 2.3 |
| HEDP | 0 | 0 | 0 | 0 | 0 | 0.13 | 0 | 0.27 | 0.16 | 0 | 0 | 0.17 |
| MGDA | 0 | 1.1 | 0 | 0 | 0 | 0 | 0 | 0 | 0 | 0 | 0 | 0 |
| Cellulase | 0-0.5 | 0-0.5 | 0-0.5 | 0-0.5 | 0-0.5 | 0-0.5 | 0-0.5 | 0-0.5 | 0-0.5 | 0-0.5 | 0-0.5 | 0-0.5 |
| Lipase | 0-0.4 | 0-0.4 | 0-0.4 | 0-0.4 | 0-0.4 | 0-0.4 | 0-0.4 | 0-0.4 | 0-0.4 | 0-0.4 | 0-0.4 | 0-0.4 |
| Mannanase | 0-0.4 | 0-0.4 | 0-0.4 | 0-0.4 | 0-0.4 | 0-0.4 | 0-0.4 | 0-0.4 | 0-0.4 | 0-0.4 | 0-0.4 | 0-0.4 |
| Protease * | 0-1.5 | 0-1.5 | 0-1.5 | 0-1.5 | 0-1.5 | 0-1.5 | 0-1.5 | 0-1.5 | 0-1.5 | 0-1.5 | 0-1.5 | 0-1.5 |
| Amylase ** | 0-0.5 | 0-0.5 | 0-0.5 | 0-0.5 | 0-0.5 | 0-0.5 | 0-0.5 | 0-0.5 | 0-0.5 | 0-0.5 | 0-0.5 | 0-0.5 |
| Na Sulfate | 30.8 | 1.3 | 33 | 11 | 22 | 3 | 51 | 4 | 6 | 57 | 38 | 37 |
| Na Chloride | 0.2 | 43 | 0.1 | 0 | 0.1 | 0.1 | 1 | 1 | 0.5 | 1.2 | 0.2 | 1 |
| optical | 0.02 | 0 | | 0.1 | 0.06 | | | 0.29 | 0.1 | 0.23 | 0.13 | 0.19 |
| brightener | | | | | | | | | | | | |
| Polymers | 1 | 0 | 0.2 | 2 | 0.5 | 3 | 2.2 | 9.2 | 2.2 | 0.7 | | 0.4 |

* preferably, the composition comprises a protease inhibitor, preferably selected from phenylboronic acid (preferably 4-FPBA) or a peptide aldehyde or a bisulfite adduct or acetal thereof (preferably a tripeptide aldehyde, preferably, Z-GAY or Z-VAL.
** preferably an amylase variant as described herein.

% wt in
formulation

| | | | | | | | | | | | | | | |
|---|---|---|---|---|---|---|---|---|---|---|---|---|---|---|
| alcohol | 5.40 | 10.80 | 12.40 | 7.30 | 1.60 | 7.60 | 3.80 | 0.30 | 13.30 | 8.00 | 5.70 | 20.00 | 9.20 | 29.00 |
| ethoxylat 7EO | | | | | | | | | | | | | | |
| Coco fatty | 2.40 | 3.10 | 3.20 | 3.20 | 3.50 | 6.40 | 2.80 | 3.00 | 1.70 | 1.80 | 2.50 | 5.00 | 8.60 | 10.40 |
| acid C12-18 | | | | | | | | | | | | | | |
| C12-C14-fatty | 5.40 | 8.80 | 7.10 | 7.10 | 5.40 | 14.00 | 2.80 | 4.50 | 3.90 | 4.10 | | 10.00 | 22.20 | |
| alcohol ether | | | | | | | | | | | | | | |
| sulfate (2 EO) | | | | | | | | | | | | | | |
| Linear alkyl | 5.50 | 0.00 | 14.50 | 15.50 | 10.70 | 0.00 | 6.30 | 5.43 | 11.45 | 5.90 | 10.10 | 10.00 | 28.00 | 27.00 |
| benzene | | | | | | | | | | | | | | |
| sulphonic | | | | | | | | | | | | | | |
| acid | | | | | | | | | | | | | | |
| 1,2 | 6.00 | 3.50 | 8.70 | 8.70 | 1.10 | 7.80 | 0.50 | | 2.50 | 0.40 | 6.00 | 10.00 | 7.00 | 7.00 |
| Propandiol | | | | | | | | | | | | | | |
| Trieth- | | | | | | | | | | 8.10 | | | | |
| anolamine | | | | | | | | | | | | | | |
| Monoeth- | | | 4.00 | 4.30 | 0.30 | | 0.40 | 1.80 | | | | | 8.00 | 7.00 |
| anolamine | | | | | | | | | | | | | | |
| NaOH | 2.20 | 1.10 | | | | 1.00 | | | 2.20 | | 3.30 | 1.50 | | |
| Glycerol | | 0.80 | 3.00 | 2.80 | | | | 0.60 | 0.20 | 1.90 | | | 7.00 | 10.00 |
| Ethanol | 2.00 | | | | 0.38 | 0.39 | | | 1.84 | | | | | |
| Na citrate | 3.00 | 2.80 | 3.40 | 2.10 | 7.40 | 5.40 | 4.60 | 3.30 | 3.30 | 1.40 | | 1.50 | | |
| Polymer | 0-5% | 0-5% | 0-5% | 0-5% | 0-5% | 0-5% | 0-5% | 0-5% | 0-5% | 0-5% | 0-5% | 0-5% | 0-5% | 0-5% |
| Protease * | 0-1% | 0-1% | 0-1% | 0-1% | 0-1% | 0-1% | 0-1% | 0-1% | 0-1% | 0-1% | 0-1% | 0-3% | 0-3% | 0-3% |
| Amylase ** | 0-0.5% | 0-0.5% | 0-0.5% | 0-0.5% | 0-0.5% | 0-0.5% | 0-0.5% | 0-0.5% | 0-0.5% | 0-0.5% | 0-0.5% | 0-0.5% | 0-0.5% | 0-0.5% |
| Cellulase | 0-0.3% | 0-0.3% | 0-0.3% | 0-0.3% | 0-0.3% | 0-0.3% | 0-0.3% | 0-0.3% | 0-0.3% | 0-0.3% | 0-0.3% | 0-0.3% | 0-0.3% | 0-0.3% |
| Lipase | 0-0.2% | 0-0.2% | 0-0.2% | 0-0.2% | 0-0.2% | 0-0.2% | 0-0.2% | 0-0.2% | 0-0.2% | 0-0.2% | 0-0.2% | 0-0.2% | 0-0.2% | 0-0.2% |
| Mannanase | 0-0.2% | 0-0.2% | 0-0.2% | 0-0.2% | 0-0.2% | 0-0.2% | 0-0.2% | 0-0.2% | 0-0.2% | 0-0.2% | 0-0.2% | 0-0.2% | 0-0.2% | 0-0.2% |
| Pectat | 0-0.3% | 0-0.3% | 0-0.3% | 0-0.3% | 0-0.3% | 0-0.3% | 0-0.3% | 0-0.3% | 0-0.3% | 0-0.3% | 0-0.3% | 0-0.3% | 0-0.3% | 0-0.3% |
| Lyase | | | | | | | | | | | | | | |
| water | to 100 | to 100 | to 100 | to 100 | to 100 | to 100 | to 100 | to 100 | to 100 | to 100 | to 100 | to 100 | to 100 | to 100 |

* preferably, the composition comprises a protease inhibitor, preferably selected from phenylboronic acid (preferably 4-FPBA) or a peptide aldehyde or a bisulfite adduct or acetal thereof (preferably a tripeptide aldehyde, preferably, Z-GAY or Z-VAL.
** preferably an amylase variant as described herein.

### Methods of Use

The present invention is also directed to the use of an amylase variant as described herein in a cleaning process such as laundry or hard surface cleaning, preferably for home care or I&I cleaning including manual and automated dish washing as well as medical device cleaning. Thus, the present invention also refers to the use of an amylase variant as described herein for providing a detergent composition with improved enzyme stability, preferably amylase stability, and/or for providing a detergent composition with improved wash performance, preferably on amylase-sensitive stains.

Thus, the present invention therefore also refers to a method for cleaning, preferably laundry or hard surface cleaning, comprising the step of contacting on object, preferably a textile or a hard surface, with a composition comprising an amylase variant as described herein, preferably wherein the composition comprises at least one additional detergent component, preferably a surfactant and/or a builder.

Further, the present invention also refers to a method for improving amylase stability in a detergent composition and/or for improving wash performance of a detergent composition, preferably on amylase-sensitive stains comprising the step of formulating an amylase variant as described herein in a detergent composition.

### Preferred Embodiments

Particularly, preferred herein is:

1. An amylase variant of a parent amylase, wherein said amylase variant comprises
(i) an amino acid substitution at two or more, three or more, four or more, five or more, six or more, or at all amino acid positions corresponding to amino acid positions selected from the group consisting of 4, 25, 176, 186, 251, 405, and 482 according to the numbering of SEQ ID NO: 3, and
(ii) at least 60%, preferably at least 91% identity, but less than 100% sequence identity with SEQ ID NO: 1, 3, 4, or with any of SEQ ID NO: 15-41, preferably with SEQ ID NO: 1 or SEQ ID NO: 3, preferably SEQ ID NO: 1, preferably wherein the parent amylase of the amylase variant is an amylase according to SEQ ID NO: 1 or SEQ ID NO: 3, preferably SEQ ID NO: 1.

2. The amylase variant according to embodiment 1, wherein said amylase variant comprises
(i) an amino acid substitution at position 4 according to the numbering of SEQ ID NO: 3,
(ii) an amino acid substitution at one or more, two or more, three or more, four or more, five or more, or at all amino acid positions corresponding to amino acid positions selected from the group consisting of 25, 176, 186, 251, 405, and 482 according to the numbering of SEQ ID NO: 3, and
(iii) at least 60%, preferably at least 91% identity, but less than 100% sequence identity with SEQ ID NO: 1, 3, 4, or with any of SEQ ID NO: 15-41, preferably with SEQ ID NO: 1 or SEQ ID NO: 3, preferably SEQ ID NO: 1, preferably wherein the parent amylase of the amylase variant is an amylase according to SEQ ID NO: 1 or SEQ ID NO: 3, preferably SEQ ID NO: 1.

3. The amylase variant according to any of the preceding embodiments, wherein said amylase variant comprises an amino acid substitution at position 25.

4. The amylase variant according to any of the preceding embodiments, wherein said amino acid substitution at position 4 is X4Q, or an amino acid substitution similar to X4Q.

5. The amylase variant according to any of the preceding embodiments, wherein said amino acid substitution at position 25 is X25H, wherein said amino acid substitution at position 176 is X176K, wherein said amino acid substitution at position 186 is X186E, wherein said amino acid substitution at position 251 is X251E, wherein said amino acid substitution at position 405 is X405M, and wherein said amino acid substitution at position 482 is X482W, or an amino acid substitution similar to these substitutions.

6. The amylase variant according to any of the preceding embodiments, wherein the parent amylase of the amylase variant is an amylase according to SEQ ID NO: 1.

7. In one embodiment, the amylase variant of the present invention comprises
(i) the amino acid substitution X4G according to the numbering of SEQ ID NO: 3,
(ii) one or more, two or more, three or more, four or more, five or more, or all of the amino acid substitutions selected from the group consisting of X25H, X176K, X186E, X251E, X405M, and X482W according to the numbering of SEQ ID NO: 3, and
(iii) at least 60%, preferably at least 91% identity, but less than 100% sequence identity with SEQ ID NO: 1, 3, 4, or with any of SEQ ID NO: 15-41, preferably with SEQ ID NO: 1 or SEQ ID NO: 3, preferably SEQ ID NO: 1.

8. The amylase variant according to any of the preceding embodiments, wherein at least 80%, preferably at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% sequence identity with SEQ ID NO: 1.

9. The amylase variant according to any of the preceding embodiments, wherein said amylase variant comprises
a) a substitution at one or more positions selected from 25, 176, and 186; preferably one or more substitutions selected from X25H, X176K, and X186E, or
b) a substitution at one or more positions selected from 251, 405, and 482, preferably one or more substitutions selected from X251E, X405M, and X482W, or
c) a substitution at one or more positions selected from 25, 176, and 186, preferably one or more substitutions selected from X25H, X176K, and X186E, and a substitution at one or more positions selected from 251, 405, and 482, preferably one or more substitutions selected from X251E, X405M, and X482W.

10. The amylase variant according to any of the preceding embodiments, wherein said variant comprises two or all of the amino acid substitutions selected from the group consisting of X25H, X176K, and X186E.

11. The amylase variant according to any of the preceding embodiments, wherein said amylase variant comprises a combination of amino acid substitutions selected from the group consisting of

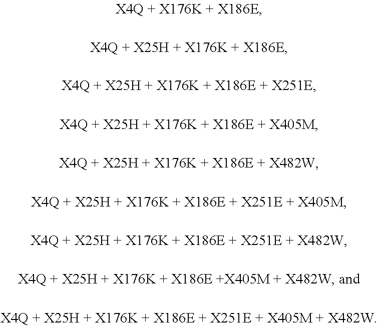

12. The amylase variant according to any of the preceding embodiments, comprising the amino acid substitutions X4Q+X25H+X176K+X186E+X251E+X405M+X482W (according to the numbering of SEQ ID NO: 3).

13. The amylase variant according to any of the preceding embodiments, further comprising a deletion of two amino acids corresponding to positions selected from the group consisting of 181, 182, 183 and 184, preferably a deletion of amino acids corresponding to positions 181 and 182, 182 and 183, or 183 and 184, preferably D183* and G184*, wherein the numbering is according to SEQ ID NO: 3.

14. The amylase variant according to any of the preceding embodiments, wherein the amylase variant has at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% sequence identity to any of SEQ ID NO: 1, 3, or 4, preferably SEQ ID NO: 1.

15. The amylase variant according to any of the preceding embodiments, wherein the variant comprises an A and B domain and a C domain, wherein the amino acid sequence of the A and B domain is at least 75%, but less than 100%, identical to the amino acid sequence of SEQ ID NO: 6 and the amino acid sequence of the C domain is at least 75%, but less than 100%, identical to the amino acid sequence of SEQ ID NO: 8.

16. The amylase variant according to any of the preceding embodiments, wherein said amylase variant comprises or consists of the amino acid sequence set forth in any of SEQ ID NO: 1, 3, 4, or in any of SEQ ID NO: 15-41, preferably SEQ ID NO: 1 or SEQ ID NO: 3, preferably SEQ ID NO: 1, with the amino acid substitution X4Q and with 1 to 6, preferably 2 to 6 or 3 to 6, such as 1, 2, 3, 4, 5, or 6 of the amino acid substitutions selected from the group consisting of X25H, X176K, X186E, X251E, X405M, and X482W, and preferably with 1 to 10, preferably 1 to 5 further conservation amino acid substitutions, and preferably a deletion of two amino acids corresponding to positions selected from the group consisting of 181, 182, 183 and 184.

17. The amylase variant according to any of the preceding embodiments, wherein the amylase variant further comprises 1 to 50, preferably 1 to 30, 1 to 25, 1 to 20, 1 to 15, 1 to 10, or 1 to 5, 2 to 30, 2 to 25, 2 to 20, 2 to 15 2 to 10, 2 to 8, or 2 to 5, preferably 3 to 30, 3 to 25, 3 to 20, 3 to 15 3 to 10, 3 to 8, or 3 to 5, preferably, 4 to 30, 4 to 25, 4 to 20, 4 to 15, 4 to 10, or 4 to 8 conservative amino acid exchanges, preferably wherein the conservative mutations are not pertaining the catalytic center of the amylase variant.

18. The amylase variant according to any of the preceding embodiments, wherein said amylase variant comprises or consists of the amino acid sequence set forth in any of SEQ ID NO: 1 with the alterations X4Q+X25H+X176K+X186E+X251E+X405M+X482W according to the numbering of SEQ ID NO. 3 and optionally with 1 to 10, preferably 1 to 5 further conservation amino acid substitutions.

19. The amylase variant according to any of the preceding embodiments, wherein the substitutions are selected from G4Q, N25H, R176K, G186E, T251E, L405M, and Y482W (according to the numbering of SEQ ID NO: 3). 20. The amylase variant according to any of the preceding embodiments, wherein the amylase variant exhibits one or more improved property, preferably relative to said parent amylase, preferably relative to the parent amylase set forth in SEQ ID NO: 1 or SEQ ID NO: 3, preferably SEQ ID NO: 1 or relative to an amylase set forth in SEQ ID NO: 33, preferably the amylase variant has an increase in stability, thermostability, storage stability, storage stability in a detergent composition, wash performance, wash performance in a laundry detergent, and/or wash performance in a dish wash detergent, preferably, the improved property is improved storage stability and/or improved wash performance, preferably improved wash performance on laundry, most preferably the improved property is improved storage stability, preferably wherein said improved property is expressed as an Improvement Factor (IF) of >1.0 and wherein preferably the Improvement Factor is equal or greater than 1.1, preferably, equal or greater than 1.2, more preferably, equal or greater than 1.3, preferably the amylase variant exhibits improved storage stability in a detergent composition, preferably relative to a reference amylase, preferably relative to said parent amylase, preferably relative to the parent amylase set forth in SEQ ID NO: 1 or SEQ ID NO: 3, preferably SEQ ID NO: 1, or relative to an amylase as set forth in SEQ ID NO: 33, preferably with an improvement factor equal or greater than 1.1, preferably, equal or greater than 1.2. more preferably, equal or greater than 1.3, preferably after storage in a detergent composition as described herein at 37° C., preferably for 14 days, 28 days, 42 days, or 56 days, preferably for 56 days, or at 40° C., preferably for 14 days, 24 days, 56 days or 84 days, preferably for 84 days, preferably as determined in ES1-C detergent as described herein, preferably containing an additional builder (preferably HEDP), preferably relative to a reference amylase, preferably relative to said parent amylase, preferably relative to the parent amylase set forth in SEQ ID NO: 1 or SEQ ID NO: 3, preferably SEQ ID NO: 1, or relative to an amylase as set forth in SEQ ID NO: 33; and/or preferably the amylase variant exhibits improved wash performance on laundry, preferably at 40° C., preferably relative to an amylase with an amino acid sequence shown in SEQ ID NO: 33.

21. A polynucleotide encoding the amylase variant according to any of the preceding embodiments.

22. A formulation comprising the amylase variant according to any one of embodiments 1 to 20 and at least one additional component, preferably a solvent.

23. The formulation according to embodiment 22, wherein the composition comprises one or more second enzyme different from the amylase variant referred to in any of the preceding claims, preferably one or more second enzyme selected from the group consisting of proteases, second amylases, lipases, cellulases, hemicellulases, mannanases, xylanases, DNases, dispersins, pectinases, oxidoreductases, and cutinases, preferably selected from protease, mannanase, and lipase, most preferably proteases.

24. The formulation according to embodiment 22 or 23, comprising an enzyme stabilizing system, preferably comprising a calcium salt and optionally in case a protease is present a protease inhibitor.

25. A detergent composition comprising the amylase variant according to any one of embodiments 1 to 20 or the formulation according to any of embodiment 22-24, preferably a laundry detergent composition or a hard surface cleaning detergent composition, most preferably laundry detergent composition.

26. The detergent composition according to embodiment 25, wherein the composition comprises one or more surfactants and/or one or more builder, preferably one or more strong sequestering builder.

27. The detergent composition according to any of embodiment 25 or 26, wherein the detergent composition comprises a component selected from the group consisting of enzyme stabilizing system, surfactant, defoamer, builder, polymer, bleaching system (bleach), rheology modifier, hydrotrope, softening agent, desiccant, whitening agent, buffer, suds suppressors, preservative, anti-corrosion additive, dyestuff and fragrance.

28. The detergent composition according to any of embodiment 25 to 27, comprising a builder, wherein the builder is selected from MDGA, GLDA, DTPMP, HEDP, and EDDS, preferably MDGA or EDDS.

29. The detergent composition according to any of embodiment 28, wherein the builder is a non-phosphate based builder.

30. The detergent composition according to any of embodiment 25 to 29, comprising a surfactant, wherein the surfactant is selected from non-ionic surfactant, anionic surfactant, cationic surfactant, amphoteric surfactant, and combinations thereof.

31. The detergent composition according to any of embodiment 25 to 30, wherein the detergent composition is devoid of anionic surfactants.

32. The detergent composition according to any of embodiment 25 to 31, wherein the surfactant and/or the builder is bio-degradable and/or bio-based.

33. The detergent composition according to any of embodiment 25 to 32, wherein the detergent composition is liquid or solid.

34. The detergent composition according to any of embodiment 25 to 33, wherein the detergent composition is in the form of a pouch.

35. The detergent composition according to any of embodiment 25 to 34, wherein the detergent composition is a liquid laundry detergent composition.

36. The detergent composition according to any of embodiment 25 to 35, wherein the detergent composition is boron-free.

37. The detergent composition according to any of embodiment 25 to 36, wherein the detergent composition does not comprise a preservative.

38. Method for cleaning comprising the step of contacting a textile or a hard surface, preferably textile, with an amylase variant according to any of embodiment 1-20 or a formulation according to any of embodiment 22-24 or a detergent composition according to any of embodiment 25-37, preferably at low temperature, preferably at 30° C. or 40° C., preferably at 30° C.

39. Using an amylase variant according to any of embodiment 1-20 or a formulation according to any of embodiment 22-24 or a detergent composition according to any of embodiment 25-37 for improving amylase stability in a detergent composition and/or for improving wash performance of a detergent composition, preferably on amylase-sensitive stains, preferably relative to an amylase set forth in SEQ ID NO: 1 or SEQ ID NO: 3, preferably SEQ ID NO: 1, or relative to an amylase as set forth in SEQ ID NO: 33, preferably 30° C. or 40° C., preferably at 40° C., preferably as determined in ES1-C detergent as described herein, preferably containing an additional builder (preferably HEDP).

### EXAMPLES

### General Methods:

### Amylase Expression

Genes coding for the amylases were cloned by restriction-ligation based standard protocols into a gram-positive expression vector comprising a promoter sequence, a sequence coding for a secretion signal peptide, and a ribosome binding site. Post reaction, the plasmid assembly mixtures were transformed into B. subtilis PY79 by established natural-competency transformation methods. Successful transformations were selected by plating on LB agar plates supplemented with 20 μg/ml kanamycin sulfate and incubating overnight at 37 degrees C. After the overnight selection, individual colonies were obtained which were then grown in a rich medium (e.g., LB broth) with 20 μg/ml kanamycin sulfate overnight at 37 degrees with shaking at 250 rpm. The following morning, cells were pelleted by centrifugation, and plasmid DNA was isolated by alkaline lysis method with a Macherey-Nagel NucleoSpin kit. The isolated DNA could then be transformed into electrocompetent Bacillus licheniformis cells.

The B. licheniformis cells were made electrocompetent by standard gram-positive electrocompetent cell preparation techniques. As an example, the cells were prepared by growing the B. licheniformis strain in an osmolyte-dense rich medium (e.g., LB broth with 0.5 M D-sorbitol) and harvesting the cells in early exponential growth phase. Cells were harvesting by chilling on ice and pelleting by centrifugation. Following the harvest, cells were washed to remove salts with an osmolyte-rich washing buffer (e.g., 10% glycerol with 0.5M D-sorbitol and 0.5M D-mannitol) by 3 cycles of suspension and pelleting by centrifugation. Finally, cells were concentrated by re-suspending in the washing buffer at 1 to 10% of the original culture volume.

Once prepared, plasmid DNA was added the B. licheniformis electrocompetent cells in a 0.2 cm Bio-Rad electroporation cuvette. The cells were electroporated with a Bio-Rad Gene Pulser Xcell per manufacturer's instructions. Cells were immediately rescued after the shock by addition of 1 ml of the osmolyte-dense rich medium. After two hours for of recovering at 37 degrees C., the successful transformants were then selected by plating on LB agar plates supplemented with 20 μg/ml kanamycin sulfate and incubating overnight at 37 degrees C.

### Amylase Expression and Protein Quantification

Single colonies of the expression strains were picked into 60 μL of rich medium (e.g. LB broth) supplemented with 20 μg/mL kanamycin sulfate in a 96-well plate (GE Life Sciences part 28403943). The cultures were grown at 37 degrees C. for 16 hours, then 15 μL of culture was used to inoculate 600 μL of defined glucose-mineral media and 20 g/mL kanamycin sulfate in a 96 well plate. The cultures were grown at 37 degrees C. for 48 hours, after which the supernatant was harvested by pelleting the cells with centrifugation and removing the residual culture liquid. Larger amounts were created by combing the supernatant of identical clones. The expression levels (concentration in mg/mL) of alpha-amylases activity were identified by LabChip (LC) analysis to allow equal dosing in laundry detergent.

### Amylase Activity Measurements

α-amylase activity can be determined by a method employing the Ethyliden-4-nitrophenyl-α-D-maltoheptaosid (EPS) as substrate. D-maltoheptaoside is a blocked oligosaccharide which can be cleaved by an endo-amylase. Following the cleavage, the α-glucosidase included in the kit to digest the substrate to liberate a free PNP molecule which has a yellow color and thus can be measured by visible spectophotometry at 405 nm. Kits containing EPS substrate and α-glucosidase is manufactured by Roche Costum Biotech (cat. No. 10880078103) or Thermo Scientific (TR25421). The slope of the time dependent absorption-curve is directly proportional to the specific activity (activity per mg enzyme) of the α-amylase in question under the given set of conditions. The residual activity was calculated by dividing the activity after storage time with the activity of the sample at time point zero. The residual activity was compared to the residual activity of the reference given in the example below.

### Example 1: Storage Stability in Formil Detergent at 37° C. For 56 Days

To assess furthermore if the improved heat stability is also connected to elevated stability in detergent, the amylases were formulated in a commercially available liquid detergent (Formil) and tested for their residual activity after 56 days of storage at 37° C.

Storage in liquid laundry detergent was done by incubating the amylases in Formil liquid detergent (Lidl, Germany). To inactive all intrinsic enzymes within the detergent, the detergent was incubated at 85° C. for 2-3 h. Inactivation was proven as no amylase activity was detectable.

| Formil formulation | wt % |
|---|---|
| Aqua | add 100 |
| MEA c10-13 alkyl benzenesulfonate | 9.0-13.0 |
| Alcohols, C12-18, ethoxylated | 10.0-12.0 |
| Propylene glycol | 4.0-8.0 |
| Sodium laureth sulfate | 4.0-8.0 |
| MEA citrate | 3.0-4.0 |
| Alcohol | 2.0-3.0 |
| MEA palm kernelate | 2.0-4.0 |
| Sodium borate | 0.1-0.3 |
| Sodium diethylenetriamine pentamethylene phosphonate | 0.1-0.4 |
| Parfum | 0.01-0.1 |
| Vinylpyrrolidone/vinylimidazole copolymer | 0.2-0.5 |
| Subtilisin | inactivated |
| Benzyl alcohol | 1.0-4.0 |
| Linalool | 0.01-0.1 |
| Hexyl cinnamal | 0.01-0.1 |
| Phenoxyethanol | 0.01-0.1 |
| Benzyl salicylate | 0.01-0.1 |
| Lipase | inactivated |
| Glycosidase | inactivated |
| Colorant | 0.01-0.1 |
| Pectate lyase | inactivated |
| Benzisothiazolinone | 0.01-0.1 |
| Methylisothiazolinone | 0.01-0.1 |

Thereafter, both amylase samples were diluted with 50 mM HEPES pH 8.0 to result in an equal active ingredient level. The detergent was dosed with a final amylase concentration of 0.12 mg/g and additionally a protease was supplemented (0.5% protease formulation comprising a protease with SEQ ID NO: 10 with R101E mutation (according to the numbering of SEQ ID NO: 11)). The mixture was incubated at 37° C. and after indicated timepoints amylase activity was measured using above-described amylase detection method.

Table 1: Storage stability of an amylase variant of the present invention in commercial product formulation “Formil” at 37° C. compared to a reference amylase (commercially available amylase (SEQ ID NO: 33)). Residual activity up to 56 days of challenge:

| | Residual activity after storage | | | | |
|---|---|---|---|---|---|
| | 0 d | 14 d | 28 d | 42 d | 56 d |
| Reference amylase: SEQ ID NO: 33 | 100% | 69% | 50% | 37% | 28% |
| Amylase variant: SEQ ID NO: 1 | 100% | 97% | 97% | 94% | 94% |
| with substitutions G4Q + N25H + | | | | | |
| R176K + G186E + T251E + | | | | | |
| L405M + Y482W (*) | | | | | |

(*) mutations in SEQ ID NO: 1 are given in the numbering of SEQ ID NO: 3

As can be derived from the table above, an amylase variant of the present invention is more stable than the reference amylase.

### Example 2: Storage Stability in Builder-Containing Detergent at 40° C. For Up to 84 Days

Table 2: An amylase variant of the invention tested versus a reference amylase (SEQ ID NO: 33) using a model detergent.

| Amylase | |
|---|---|
| Reference amylase | SEQ ID NO: 33 |
| Amylase variant | SEQ ID NO: 1 with substitutions |
| | G4Q + N25H + R176K + G186E + |
| | T251E + L405M + W439K + Y482W (*) |

(*) Amylase variant of SEQ ID NO: 1 with the mutations given in the numbering of SEQ ID NO: 3.

The amylases were formulated in a liquid model detergent (ES1-C) and tested for their residual activity after 84 days of storage at 40° C. Final dosing was 0.1 g/L of amylase. To assess the stability of the amylases in presence of strong chelators, HEDP and/or CaCl₂) was supplemented as indicated in the table. No additional Ca2+was supplemented by the enzymes.

TABLE 3
ES1-C formulation:

| ES1-C | | wt % |
|---|---|---|
| Maranil DBS/LC | linear alkylbenzene sulfonate, anionic | 5.5 |
| | surfactant | |
| Edenor coco fatty acid | C₁₂-C₁₈ coco fatty acid | 2.4 |
| Lutensol AO7 | alkyl polyethyleneglycol ether, non-ionic | 5.5 |
| | surfactant | |
| Texapon N70 | sodium laureth sulfate 2EO, anionic | 5.5 |
| | surfactant | |
| 1,2 propylene glycol | | 6.0 |
| C₂H₅OH | | 2.0 |
| NaOH | | 2.2 |
| Citrate | | 3.0 |
| pH | | 8-8.5 |
| H₂O | up to 100% | |

After indicated timepoints sample were taken and residual activity was measured using EPS as substrate as indicated above. The residual activity was calculated by dividing the activity after storage time with the activity of the sample at time point zero. The residual activity was compared to the residual activity of a reference amylase (SEQ ID NO: 33).

TABLE 4

| | CaCl2 | HEDP | Day | Day | Day | Day | Day |
|---|---|---|---|---|---|---|---|
| Amylase | (ppm) | (%) | 0 | 14 | 24 | 56 | 84 |
| SEQ ID NO: 33 | 0 | 0 | 100% | 79% | 80% | 54% | 55% |
| SEQ ID NO: 33 | 0 | 0.05 | 100% | 80% | 79% | 50% | 43% |
| SEQ ID NO: 33 | 150 | 0 | 100% | 86% | 86% | 61% | 67% |
| SEQ ID NO: 33 | 150 | 0.05 | 100% | 83% | 69% | 40% | 39% |
| Amylase variant | 0 | 0 | 100% | 99% | 95% | 87% | 91% |
| Amylase variant | 0 | 0.05 | 100% | 95% | 92% | 81% | 81% |
| Amylase variant | 150 | 0 | 100% | 104% | 101% | 87% | 85% |
| Amylase variant | 150 | 0.05 | 100% | 93% | 97% | 79% | 86% |

As can be derived from the table above, an amylase variant of the present invention is more stable than the reference amylase.

### Example 3: Wash Performance of Improved Amylases

Determination of the wash performance was performed using a Launder-o-meter: Several soiled swatches were washed together with cotton ballast fabric and 20 steel balls at 40° C. in a liquid laundry formulation. After the wash, the fabrics were rinsed, spin-, and air-dried. The washing performance was determined by measuring the CIELab values of the soiled fabrics before and after wash using the MACH5 multi area color measurement. For data evaluation, ΔΕ was calculated between unwashed and washed stains and ΔΔΕ was calculated between detergent with enzymes and detergent without enzymes. The data represent average values of two separate experiments.

TABLE 6
Washing conditions

| | |
|---|---|
| Test equipment | Launder-o-meter, LP2 Typ, SDL Atlas Inc., USA |
| Washing liquor | 250 ml |
| Washing time and | 20 min at 40° C. |
| temperature | |
| Detergent dosage | 3.3 g/L |
| Fabric/liquor ratio | 1:12.5 |
| Washing cycles | 1 |
| Water hardness | 2.5 mmol/l Ca2+:Mg2+:HCO3− 4:1:8 |
| Ballast fabric | 10 g cotton fabric |
| Soiled fabric | 2 pieces 5 × 5 cm CFT-CS 28 1) |
| | 2 pieces 5 × 5 cm EMPA 161 2) |
| | 2 pieces 5 × 5 cm CFT-CS 129 1) |
| Laundry formulation | Single unit dose formulation: |
| | Maranil DBS/LC: 4.9 w/w % |
| | Glycerol: 2 w/w % |
| | 1,2 Propylene glycol: 1.8 w/w % |
| | Edenor coco fatty acid K12-18: 1.6 w/w % |
| | MEA: 1.1 w/w % |
| | Lutensol AO 7: 5.6 w/w % |
| | Water up to 90% |
| Test equipment | Launder-o-meter, LP2 Typ, SDL Atlas Inc., USA |
| Enzyme concentrations | 0.01-0.1 ppm (referring to active enzyme) |
| in wash liquor | |

1) CFT-CS 28, CFT-CS 129 Producer: Center for Testmaterials BV, NL-3130 AC Vlaardingen
2) EMPA 161, Producer: Swissatest Testmaterialien AG, Mövenstrasse 12, 9015 St. Gallen, Schweiz

TABLE 7
Fresh Performance in Launder-o-meter. The data show the
summarized performance over three stains at 40° C.

| Total ΔΔE | 0.01 ppm | 0.05 ppm |
|---|---|---|
| Reference amylase: SEQ ID NO: 33 | 39.5 | 59.5 |
| Amylase variant: SEQ ID NO: 1 with | 41.2 | 61.9 |
| substitutions G4Q + N25H + R176K + | | |
| G186E + T251E + L405M + Y482W (*) | | |

(*) with the mutations in SEQ ID NO: 1 are given in the numbering of SEQ ID NO: 3

As can be derived from the table above, an amylase variant of the present invention provides better cleaning result compared to the reference amylase.

### Determination of Wash Performance in Full Scale Washing Machines:

A multi-stain monitor was washed in full scale washing machines in presence with cotton ballast fabric at 40° C. in a liquid laundry formulation with different amylases. Each stain was washed in quadruplicates (2 machines with two pieces per stain per machine). After the wash, the stain monitor was air-dried. The washing performance was determined by measuring the CIELab values of the soiled fabrics before and after wash using the MACH5 multi area color measurement. For data evaluation mean values were determined, ΔY was calculated between unwashed and washed stains and ΔΔY was calculated between detergent with enzymes and detergent without enzymes.

TABLE 8
Washing conditions

| | |
|---|---|
| Test equipment | Miele W 1935 WPS WTL |
| Washing liquor | 15 l |
| Program | Cotton Short, 40° C. |
| Detergent dosage | 3.3 g/L |
| Washing cycles | 1 |
| Water hardness | 2.5 mmol/l Ca2+:Mg2+:HCO3− 4:1:8 |
| Ballast fabric | 3.5 kg huckaback towels |
| Soiled fabric | 2 pieces CFT CS 26 1) |
| | 2 pieces CFT CS 27 1) |
| | 2 pieces CFT CS 28 1) |
| | 2 pieces CFT CS 29 1) |
| | 2 pieces CFT CS 126 1) |
| | 2 pieces CFT CS 127 1) |
| | 2 pieces CFT CS 128 1) |
| | 2 pieces CFT CS 129 1) |
| | 2 pieces EMPA 161 2) |
| | 2 pieces EMPA 162 2) |
| | 2 pieces W-10R 1) |
| Laundry formulation | ES1-C formulation as given in Table 3 |
| Enzyme concentrations | Amylase: 0.01-0.05 ppm |

1) obtained from Center for Testmaterials BV, NL-3130 AC Vlaardingen
2) obtained from Swissatest Testmaterialien AG, Mövenstrasse 12, 9015 St. Gallen, Schweiz

TABLE 9
Full Scale wash performance in ES1C formulation at 40°
C. The data show the summarized performance over all
11 stains stated as ΔΔY:

| | 0.01 ppm |
|---|---|
| Reference amylase: SEQ ID NO: 33 | 130.2 |
| Amylase variant: SEQ ID NO: 1 with | 138.0 |
| substitutions G4Q + N25H + R176K + | |
| G186E + T251E + L405M + Y482W (*) | |

(*) with the mutations in SEQ ID NO: 1 are given in the numbering of SEQ ID NO: 3

As can be derived from the table above, an amylase variant of the present invention provides better cleaning result compared to the reference amylase.

1. An amylase variant of a parent amylase, wherein said amylase variant
(i) comprises an amino acid substitution at position 4 according to the numbering of SEQ ID NO: 3,
(ii) comprises an amino acid substitution at one or more amino acid positions corresponding to amino acid positions selected from the group consisting of 25, 176, 186, 251, 405, and 482 according to the numbering of SEQ ID NO: 3, and
(iii) has at least 60%, but less than 100% sequence identity with SEQ ID NO: 1, 3, 4, or with any of SEQ ID NO: 15-41.

2. The amylase variant according to claim 1, wherein said amylase variant comprises an amino acid substitution at position 25.

3. The amylase variant according to claim 1, wherein said amino acid substitution at position 4 is X4Q.

4. The amylase variant according to claim 1, wherein said amino acid substitution at position 25 is X25H, wherein said amino acid substitution at position 176 is X176K, wherein said amino acid substitution at position 186 is X186E, wherein said amino acid substitution at position 251 is X251E, wherein said amino acid substitution at position 405 is X405M, and wherein said amino acid substitution at position 482 is X482W.

5. The amylase variant according to claim 1,
wherein said amylase variant comprises
a) a substitution at one or more positions selected from 25, 176, and 186; optionally one or more substitutions selected from X25H, X176K, and X186E, or
b) a substitution at one or more positions selected from 251, 405, and 482; optionally one or more substitutions selected from X251E, X405M, and X482W, or
c) a substitution at one or more positions selected from 25, 176, and 186, optionally one or more substitutions selected from X25H, X176K, and X186E, and a substitution at one or more positions selected from 251, 405, and 482, optionally one or more substitutions selected from X251E, X405M, and X482W.

6. The amylase variant according to claim 1,
wherein said amylase variant comprises a combination of amino acid substitutions selected from the group consisting of

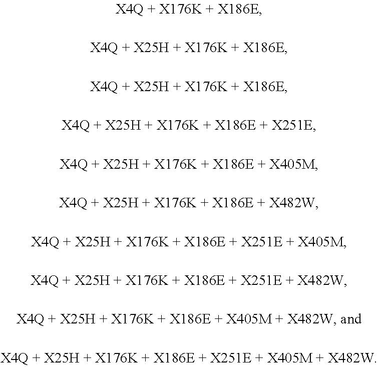

7. The amylase variant according to claim 1, further comprising a deletion at one or more amino acids corresponding to positions selected from the group consisting of 181, 182, 183 and 184, optionally a deletion of two or more amino acids corresponding to positions selected from the group consisting of 181, 182, 183 and 184, optionally a deletion of amino acids corresponding to positions 181 and 182, 182 and 183, or 183 and 184, wherein the numbering is according to the amino acid sequence set forth in SEQ ID NO: 3.

8. The amylase variant according to claim 1, wherein the amylase variant has at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, or at least 99.5%, but less than 100% sequence identity to any of SEQ ID NO: 1, 3, 4, or to any of SEQ ID NO: 15-41.

9. The amylase variant according to claim 1, wherein the amylase variant exhibits improved storage stability and/or improved wash performance, optionally the amylase variant exhibits improved storage stability in a detergent composition.

10. A polynucleotide encoding the amylase variant according to claim 1.

11. A composition comprising the amylase variant according to claim 1 and at least one additional component.

12. The composition according to claim 11, wherein the composition comprises one or more second enzyme different from the amylase variant, optionally one or more second enzyme selected from the group consisting of proteases, second amylases, lipases, cellulases, hemicellulases, mannanases, xylanases, DNases, dispersins, pectinases, oxidoreductases, and cutinases.

13. The composition according to claim 11, wherein the composition is a detergent composition, optionally a laundry detergent composition or a hard surface cleaning detergent composition.

14. The composition according to claim 11, wherein composition comprises one or more surfactants and/or one or more builders, optionally one or more strong sequestering builders.

15. The amylase variant according to claim 1, comprising an amino acid substitution at two or more amino acid positions corresponding to amino acid positions selected from the group consisting of 25, 176, 186, 251, 405, and 482 according to the numbering of SEQ ID NO: 3.


SEQUENCE LISTING (41 sequences)

SEQ ID NO: 1 - protein, 483 aa, synthetic construct
  REGION 1..483: note = Artificial Sequence
HHNGTNGTMMQYFEWYLPNDGNHWNRLRSDASNLKDKGITAVWIPPAWKGASQNDVGYGAYDLYDLGEFNQKGTVRTKYGTRNQLQAAVTALKSNGIQVYGDVVMNHKGGADATEWVRAVEVNPSNRNQEVSGDYTIEAWTKFDFPGRGNTHSNFKWRWYHFDGVDWDQSRQLQNRIYKFRGKGWDWEVDTENGNYDYLMYADIDMDHPEVVNELRNWGVWYTNTLGLDGFRIDAVKHIKYSFTRDWLTHVRNTTGKNMFAVAEFWKNDIGAIENYLSKTNWNHSVFDVPLHYNLYNASRSGGNYDMRQIFNGTVVQRHPTHAVTFVDNHDSQPEEALESFVEEWFKPLAYALTLTRDQGYPSVFYGDYYGIPTHGVPAMKSKIDPILEARQKYAYGTQRDYLDHPDVIGWTREGDGVHADSGLATLMSDGPGGSKWMEVGKNNAGEVWYDITGNQTNTVTINKDGWGQFHVSGGSVSIYVQQ

SEQ ID NO: 2 - DNA, 1449 bp, synthetic construct
  source 1..1449: mol_type = other DNA
  misc_feature 1..1449: note = Artificial Sequence
catcacaatggcacaaacggtaccatgatgcaatattttgagtggtacctcccaaatgacggaaatcattggaacagattgcgctctgatgcaagcaaccttaaagataagggcataacagctgtttggattccgccagcatggaaaggcgccagtcaaaacgacgtcggatatggagcgtacgatctgtatgatttaggcgagtttaatcagaaaggcacagtaagaacgaaatatggaaccaggaatcaacttcaggcggctgtgacggccctcaagtctaatggcattcaggtgtatggcgacgttgtgatgaaccacaaaggaggagctgatgcgacagaatgggttagagcagtcgaagtaaatcctagcaatcgaaaccaggaggtgagcggtgattacacaatcgaggcatggaccaaattcgactttcctggacgtggcaacacacatagtaattttaagtggagatggtatcacttcgatggcgtagactgggatcaatctagacagttacagaaccggatctacaaatttcgcggaaaaggctgggattgggaagttgatacggagaacggcaattatgattacttgatgtacgcagatatcgacatggatcatccggaagtggtcaatgagttacgtaattggggcgtctggtacactaacacactgggccttgacggattccgcattgatgcggttaaacatatcaaatattcgtttacgcgcgactggttaactcatgtccggaatacaacaggcaagaatatgtttgctgttgcagaattttggaaaaacgatatcggcgcgattgaaaattatctttcaaagactaactggaatcattccgtattcgacgtgcctttgcactacaacctgtataatgcgtcgcgctccggcggtaactatgacatgcgacaaatcttcaacggcacagtcgttcagcgtcaccccacacacgctgttacatttgtagacaaccatgacagccaaccggaggaagctcttgaaagcttcgtagaagaatggtttaagcctctggcctatgcgctgacgcttacgcgggatcaaggatacccgagcgtgttttatggagattactatggcattccgacgcatggcgtgccagccatgaaatcaaaaattgatccgatactcgaagccagacaaaaatatgcatatggtactcaacgggattatttggaccatccggatgtcattggttggacccgtgaaggagatggagtacatgctgactctggcttagccacactgatgtccgatggtcctggaggctcaaaatggatggaagtgggtaagaacaatgcaggtgaagtttggtatgatattaccggcaatcagacgaatacggttactattaataaagatggttggggacaatttcatgtctcaggcggctctgtctcaatctatgttcagcag

SEQ ID NO: 3 - protein, 485 aa, synthetic construct
  REGION 1..485: note = Artificial Sequence
HHNGTNGTMMQYFEWYLPNDGNHWNRLRSDASNLKDKGITAVWIPPAWKGASQNDVGYGAYDLYDLGEFNQKGTVRTKYGTRNQLQAAVTALKSNGIQVYGDVVMNHKGGADATEWVRAVEVNPSNRNQEVSGDYTIEAWTKFDFPGRGNTHSNFKWRWYHFDGVDWDQSRQLQNRIYKFRGDGKGWDWEVDTENGNYDYLMYADIDMDHPEVVNELRNWGVWYTNTLGLDGFRIDAVKHIKYSFTRDWLTHVRNTTGKNMFAVAEFWKNDIGAIENYLSKTNWNHSVFDVPLHYNLYNASRSGGNYDMRQIFNGTVVQRHPTHAVTFVDNHDSQPEEALESFVEEWFKPLAYALTLTRDQGYPSVFYGDYYGIPTHGVPAMKSKIDPILEARQKYAYGKQNDYLDHHNMIGWTREGNTAHPNSGLATIMSDGPGGNKWMYVGRNKAGQVWRDITGNRSGTVTINADGWGNFSVNGGSVSIWVNN

SEQ ID NO: 4 - protein, 483 aa, synthetic construct
  REGION 1..483: note = Artificial Sequence
HHNGTNGTMMQYFEWYLPNDGNHWNRLRSDASNLKDKGITAVWIPPAWKGASQNDVGYGAYDLYDLGEFNQKGTVRTKYGTRNQLQAAVTALKSNGIQVYGDVVMNHKGGADATEWVRAVEVNPSNRNQEVSGDYTIEAWTKFDFPGRGNTHSNFKWRWYHFDGVDWDQSRQLQNRIYKFRGKGWDWEVDTENGNYDYLMYADIDMDHPEVVNELRNWGVWYTNTLGLDGFRIDAVKHIKYSFTRDWLTHVRNTTGKNMFAVAEFWKNDIGAIENYLSKTNWNHSVFDVPLHYNLYNASRSGGNYDMRQIFNGTVVQRHPTHAVTFVDNHDSQPEEALESFVEEWFKPLAYALTLTRDQGYPSVFYGDYYGIPTHGVPAMKSKIDPILEARQKYAYGKQNDYLDHHNMIGWTREGNTAHPNSGLATIMSDGPGGNKWMYVGRNKAGQVWRDITGNRSGTVTINADGWGNFSVNGGSVSIWVNN

SEQ ID NO: 5 - protein, 486 aa, synthetic construct
  REGION 1..486: note = Artificial Sequence
ATINNGTLMQYFEWYAPNDGNHWKRLHTDAGNLAQKGITSVWIPPAYKGTTQNDVGYGAYDLYDLGEFNQKGTVRTKYGTKAQLKSAIEALHKQNINVYGDVVMNHKGGADYTETVTAVEVDRNNRNIEVSGDYEINAWTGFNFPGRGDTHSNFKWKWYHFDGTDWDEGRKLNRIYKFRGIGKAWDWEVSSENGNYDYLMYADLDFDHPDVANEMKNWGTWYANELNLDGFRLDAVKHIDHEYLRDWVNHVRQQTGKEMFTVAEYWQNDIQTLNNYLAKVNYNQSVFDAPLHYNFHYASKGNGNYDMRNILNGTVMQNHPALAVTLVENHDSQPGQSLESVVSPWFKPLAYAFILTRAEGYPSVFYGDYYGTSGNSSYEIPALKDKIDPILMARKNFAYGTQRDYLDHPDVIGWTREGDGVHADSGLATLISDGPGGSKWMEVGKNNAGEVWYDMTGNQTNTVTINKDGWGQFHVSGGSVSIYVQQ

SEQ ID NO: 6 - protein, 399 aa, synthetic construct
  REGION 1..399: note = Artificial Sequence
HHNGTNGTMMQYFEWYLPNDGNHWNRLRSDASNLKDKGITAVWIPPAWKGASQNDVGYGAYDLYDLGEFNQKGTVRTKYGTRNQLQAAVTALKSNGIQVYGDVVMNHKGGADATEWVRAVEVNPSNRNQEVSGDYTIEAWTKFDFPGRGNTHSNFKWRWYHFDGVDWDQSRQLQNRIYKFRGDGKGWDWEVDTENGNYDYLMYADIDMDHPEVVNELRNWGVWYTNTLGLDGFRIDAVKHIKYSFTRDWLTHVRNTTGKNMFAVAEFWKNDIGAIENYLSKTNWNHSVFDVPLHYNLYNASRSGGNYDMRQIFNGTVVQRHPTHAVTFVDNHDSQPEEALESFVEEWFKPLAYALTLTRDQGYPSVFYGDYYGIPTHGVPAMKSKIDPILEARQKYAYG

SEQ ID NO: 7 - protein, 86 aa, synthetic construct
  REGION 1..86: note = Artificial Sequence
KQNDYLDHHNMIGWTREGNTAHPNSGLATIMSDGPGGNKWMYVGRNKAGQVWRDITGNRSGTVTINADGWGNFSVNGGSVSIWVNN

SEQ ID NO: 8 - protein, 86 aa, synthetic construct
  REGION 1..86: note = Artificial Sequence
TQRDYLDHPDVIGWTREGDGVHADSGLATLISDGPGGSKWMEVGKNNAGEVWYDMTGNQTNTVTINKDGWGQFHVSGGSVSIYVQQ

SEQ ID NO: 9 - protein, 400 aa, synthetic construct
  REGION 1..400: note = Artificial Sequence
ATINNGTLMQYFEWYAPNDGNHWKRLHTDAGNLAQKGITSVWIPPAYKGTTQNDVGYGAYDLYDLGEFNQKGTVRTKYGTKAQLKSAIEALHKQNINVYGDVVMNHKGGADYTETVTAVEVDRNNRNIEVSGDYEINAWTGFNFPGRGDTHSNFKWKWYHFDGTDWDEGRKLNRIYKFRGIGKAWDWEVSSENGNYDYLMYADLDFDHPDVANEMKNWGTWYANELNLDGFRLDAVKHIDHEYLRDWVNHVRQQTGKEMFTVAEYWQNDIQTLNNYLAKVNYNQSVFDAPLHYNFHYASKGNGNYDMRNILNGTVMQNHPALAVTLVENHDSQPGQSLESVVSPWFKPLAYAFILTRAEGYPSVFYGDYYGTSGNSSYEIPALKDKIDPILMARKNFAYG

SEQ ID NO: 10 - protein, 269 aa, Bacillus lentus
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGADGRGAISSIAQGLEWAGNNGMHVANLSLGSPSPSATLEQAVNSATSRGVLVVAASGNSGASSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 11 - protein, 275 aa, Bacillus amyloliquefaciens [sequence omitted]

SEQ ID NO: 12 - protein, 269 aa, Bacillus gibsonii
QQTVPWGITRVQAPTVHNRGITGSGVKVAILDTGIAQHSDLTIRGGASFVPGESTTADLNGHGTHVAGTVAALNNSIGVIGVAPSADLYAVKVLGANGRGSVSGIAQGLEWAATNNMHIANMSLGSDAPSTTLERAVNYATSRGVLVIAATGNNGTGSIGYPARYANAMAVGATDQNNRRASFSQYGTGIDIVAPGVGIQSTYLNNSYASMPGTSMATPHVAGVAALVKQKNPSWNATQIRNHLKNTATNLGNSSQFGSGLVNADAATR

SEQ ID NO: 13 - protein, 275 aa, Bacillus pumilus
AQTVPYGIPQIKAPAVHAQGYKGANVKVAVLDTGIHAAHPDLNVAGGASFVPSEPNATQDFQSHGTHVAGTIAALDNTIGVLGVAPSASLYAVKVLDRNGDGQYSWIISGIEWAVANNMDVINMSLGGPNGSTALKNAVDTANNRGVVVVAAAGNSGSFGSTSTVGYPAKYDSTIAVANVNSNNVRNSSSSAGPELDVSAPGTSILSTVPSSGYTSYTGTSMASPHVAGAAALILSKYPNLSTSQVRQRLENTATPLGDSFYYGKGLINVQAASN

SEQ ID NO: 14 - protein, 311 aa, synthetic construct [sequence omitted]

SEQ ID NO: 15 - protein, 483 aa, Bacillus sp.
HHNGTNGTMMQYFEWHLPNDGNHWNRLRDDASNLRNRGITAIWIPPAWKGTSQNDVGYGAYDLYDLGEFNQKGTVRTKYGTRSQLESAIHALKNNGVQVYGDVVMNHKGGADATENVLAVEVNPNNRNQEISGDYTIEAWTKFDFPGRGNTYSDFKWRWYHFDGVDWDQSRQFQNRIYKFRGKAWDWEVDSENGNYDYLMYADVDMDHPEVVNELRRWGEWYTNTLNLDGFRIDAVKHIKYSFTRDWLTHVRNATGKEMFAVAEFWKNDLGALENYLNKTNWNHSVFDVPLHYNLYNASNSGGNYDMAKLLNGTVVQKHPMHAVTFVDNHDSQPGESLESFVQEWFKPLAYALILTREQGYPSVFYGDYYGIPTHSVPAMKAKIDPILEARQNFAYGTQHDYFDHHNIIGWTREGNTTHPNSGLATIMSDGPGGEKWMYVGQDKAGQVWHDITGNKPGTVTINADGWANFSVNGGSVSIWVKR

SEQ ID NO: 16 - protein, 485 aa, synthetic construct
  REGION 1..485: note = Artificial Sequence
HHDGTNGTIMQYFEWNVPNDGQHWNRLHNNAQNLKNAGITAIWIPPAWKGTSQNDVGYGAYDLYDLGEFNQKGTVRTKYGTKAELERAIRSLKANGIQVYGDVVMNHKGGADFTERVQAVEVNPQNRNQEVSGTYQIEAWTGFNFPGRGNQHSSFKWRWYHFDGTDWDQSRQLANRIYKFRGDGKAWDWEVDTENGNYDYLMYADVDMDHPEVINELNRWGVWYANTLNLDGFRLDAVKHIKFSFMRDWLGHVRGQTGKNLFAVAEYWKNDLGALENYLSKTNWTMSAFDVPLHYNLYQASNSSGNYDMRNLLNGTLVQRHPSHAVTFVDNHDTQPGEALESFVQGWFKPLAYATILTREQGYPQVFYGDYYGIPSDGVPSYRQQIDPLLKARQQYAYGTQHDYLDNQDVIGWTREGDSAHAGSGLATVMSDGPGGSKTMYVGTAHAGQVFKDITGNRTDTVTINSAGNGTFPCNGGSVSIWVKQ

SEQ ID NO: 17 - protein, 485 aa, Cytophaga sp.
AATNGTMMQYFEWYVPNDGQQWNRLRTDAPYLSSVGITAVWTPPAYKGTSQADVGYGPYDLYDLGEFNQKGTVRTKYGTKGELKSAVNTLHSNGIQVYGDVVMNHKAGADYTENVTAVEVNPSNRNQETSGEYNIQAWTGFNFPGRGTTYSNFKWQWFHFDGTDWDQSRSLSRIFKFRGTGKAWDWEVSSENGNYDYLMYADIDYDHPDVVNEMKKWGVWYANEVGLDGYRLDAVKHIKFSFLKDWVDNARAATGKEMFTVGEYWQNDLGALNNYLAKVNYNQSLFDAPLHYNFYAASTGGGYYDMRNILNNTLVASNPTKAVTLVENHDTQPGQSLESTVQPWFKPLAYAFILTRSGGYPSVFYGDMYGTKGTTTREIPALKSKIEPLLKARKDYAYGTQRDYIDNPDVIGWTREGDSTKAKSGLATVITDGPGGSKRMYVGTSNAGEIWYDLTGNRTDKITIGSDGYATFPVNGGSVSVWVQQ

SEQ ID NO: 18 - protein, 484 aa, Bacillus sp.
NTAPINETMMQYFEWDLPNDGTLWTKVKNEAANLSSLGITALWLPPAYKGTSQSDVGYGVYDLYDLGEFNQKGTIRTKYGTKTQYIQAIQAAKAAGMQVYADVVFNHKAGADGTEFVDAVEVDPSNRNQETSGTYQIQAWTKFDFPGRGNTYSSFKWRWYHFDGTDWDESRKLNRIYKFRSTGKAWDWEVDTENGNYDYLMFADLDMDHPEVVTELKNWGTWYVNTTNIDGFRLDAVKHIKYTFFPDWLTYVRNQTGKNLFAVGEFWSYDVNKLHNYITKTNGSMSLFDAPLHNNFYTASKSSGYFDMRYLLNNTLMKDQPSLAVTLVDNHDTQPGQSLQSWVEPWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 19 - protein, 482 aa, synthetic construct
  REGION 1..482: note = Artificial Sequence
HHNGTNGTMMQYFEWHLPNDGNHWNRLRDDASNLRNRGITAIWIPPAWKGTSQNDVGYGAYDLYDLGEFNQKGTVRTKYGTRSQLESAIHALKNNGVQVYGDVVMNHKAGADGTEFVDAVEVDPSNRNQETSGTYQIQAWTKFDFPGRGNTYSSFKWRWYHFDGTDWDESRKLNRIYKFTGKAWDWEVDTENGNYDYLMFADLDMDHPEVVNELRRWGEWYTNTLNLDGFRIDAVKHIKYSFTRDWLTHVRNATGKEMFAVAEFWKNDLGALENYLNKTNWNHSVFDVPLHYNLYNASNSGGNYDMAKLLNGTVVQKHPMHAVTFVDNHDSQPGESLESFVQEWFKPLAYALILTREQGYPSVFYGDYYGIPTHSVPAMKAKIDPILEARQNFAYGTQHDYFDHHNIIGWTREGNTTHPNSGLATIMSDGPGGEKWMYVGQNKAGQVWHDITGNKPGTVTINADGWANFSVNGGSVSIWVKR

SEQ ID NO: 20 - protein, 485 aa, Bacillus sp.
HHNGTNGTMMQYFEWHLPNDGNHWNRLRDDASNLRNRGITAIWIPPAWKGTSQNDVGYGAYDLYDLGEFNQKGTVRTKYGTRSQLESAIHALKNNGVQVYGDVVMNHKGGADATENVLAVEVNPNNRNQEISGDYTIEAWTKFDFPGRGNTYSDFKWRWYHFDGVDWDQSRQFQNRIYKFRGDGKAWDWEVDSENGNYDYLMYADVDMDHPEVVNELRRWGEWYTNTLNLDGFRIDAVKHIKYSFTRDWLTHVRNATGKEMFAVAEFWKNDLGALENYLNKTNWNHSVFDVPLHYNLYNASNSGGNYDMAKLLNGTVVQKHPMHAVTFVDNHDSQPGESLESFVQEWFKPLAYALILTREQGYPSVFYGDYYGIPTHSVPAMKAKIDPILEARQNFAYGTQHDYFDHHNIIGWTREGNTTHPNSGLATIMSDGPGGEKWMYVGQDKAGQVWHDITGNKPGTVTINADGWANFSVNGGSVSIWVKR

SEQ ID NO: 21 - protein, 485 aa, Bacillus sp.
HHNGTNGTMMQYFEWYLPNDGNHWNRLNSDASNLKSKGITAVWIPPAWKGASQNDVGYGAYDLYDLGEFNQKGTVRTKYGTRSQLQAAVTSLKNNGIQVYGDVVMNHKGGADATEMVRAVEVNPNNRNQEVTGEYTIEAWTRFDFPGRGNTHSSFKWRWYHFDGVDWDQSRRLNNRIYKFRGHGKAWDWEVDTENGNYDYLMYADIDMDHPEVVNELRNWGVWYTNTLGLDGFRIDAVKHIKYSFTRDWINHVRSATGKNMFAVAEFWKNDLGAIENYLQKTNWNHSVFDVPLHYNLYNASKSGGNYDMRNIFNGTVVQRHPSHAVTFVDNHDSQPEEALESFVEEWFKPLAYALTLTREQGYPSVFYGDYYGIPTHGVPAMRSKIDPILEARQKYAYGKQNDYLDHHNIIGWTREGNTAHPNSGLATIMSDGAGGSKWMFVGRNKAGQVWSDITGNRTGTVTINADGWGNFSVNGGSVSIWVNK

SEQ ID NO: 22 - protein, 514 aa, Bacillus sp.
MKQQKRLYARLLTLLFALIFLLPHSAAAAHHNGTNGTMMQYFEWHLPNDGNHWNRLRDDASNLRNRGITAIWIPPAWKGTSQNDVGYGAYDLYDLGEFNQKGTVRTKYGTRSQLESAIHALKNNGVQVYGDVVMNHKGGADATENVLAVEVNPNNRNQEISGDYTIEAWTKFDFPGRGNTYSDFKWRWYHFDGVDWDQSRQFQNRIYKFRGDGKAWDWEVDSENGNYDYLMYADVDMDHPEVVNELRRWGEWYTNTLNLDGFRIDAVKHIKYSFTRDWLTHVRNATGKEMFAVAEFWKNDLGALENYLNKTNWNHSVFDVPLHYNLYNASNSGGNYDMAKLLNGTVVQKHPMHAVTFVDNHDSQPGESLESFVQEWFKPLAYALILTREQGYPSVFYGDYYGIPTHSVPAMKAKIDPILEARQNFAYGTQHDYFDHHNIIGWTREGNTTHPNSGLATIMSDGPGGEKWMYVGQDKAGQVWHDITGNKPGTVTINADGWANFSVNGGSVSIWVKR

SEQ ID NO: 23 - protein, 582 aa, Bacillus sp.
NTAPINETMMQYFEWDLPNDGTLWTKVKNEAANLSSLGITALWLPPAYKGTSQSDVGYGVYDLYDLGEFNQKGTIRTKYGTKTQYIQAIQAAKAAGMQVYADVVFNHKAGADGTEFVDAVEVDPSNRNQETSGTYQIQAWTKFDFPGRGNTYSSFKWRWYHFDGTDWDESRKLNRIYKFRSTGKAWDWEVDTENGNYDYLMFADLDMDHPEVVTELKNWGTWYVNTTNIDGFRLDAVKHIKYSFFPDWLTYVRNQTGKNLFAVGEFWSYDVNKLHNYITKTNGSMSLFDALHNNFYTASKSSGYFDMRYLLNNTLMKDQPSLAVTLVDNHDTQPGQSLQSWVEPWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAKTSNVTFTVNNATTTSGQNVYVVANIPELGNWNTANAIKMNPSSYPTWKATIALPQGKAIEFKFIKKDQAGNVIWESTSNRTYTVPFSSTGSYTASWNVP

SEQ ID NO: 24 - protein, 480 aa, Bacillus sp.
DGLNGTMMQYYEWHLENDGQHWNRLHDDAAALSDAGITAIWIPPAYKGNSQADVGYGAYDLYDLGEFNQKGTVRTKYGTKAQLERAIGSLKSNDINVYGDVVMNHKMGADFTEAVQAVQVNPTNRWQDISGAYTIDAWTGFDFSGRNNAYSDFKWRWFHFNGVDWDQRYQENHIFRFANTNWNWRVDEENGNYDYLLGSNIDFSHPEVQDELKDWGSWFTDELDLDGYRLDAIKHIPFWYTSDWVRHQRNEADQDLFVVGEYWKDDVGALEFYLDEMNWEMSLFDVPLNYNFYRASQQGGSYDMRNILRGSLVEAHPMHAVTFVDNHDTQPGESLESWVADWFKPLAYATILTREGGYPNVFYGDYYGIPNDNISAKKDMIDELLDARQNYAYGTQHDYFDHWDVVGWTREGSSSRPNSGLATIMSNGPGGSKWMYVGRQNAGQTWTDLTGNNGASVTINGDGWGEFFTNGGSVSVYVNQ

SEQ ID NO: 25 - protein, 483 aa, Bacillus halmapalus
HHNGTNGTMMQYFEWHLPNDGNHWNRLRDDASNLRNRGITAIWIPPAWKGTSQNDVGYGAYDLYDLGEFNQKGTVRTKYGTRSQLESAIHALKNNGVQVYGDVVMNHKGGADATENVLAVEVNPNNRNQEISGDYTIEAWTKFDFPGRGNTYSDFKWRWYHFDGVDWDQSRQFQNRIYKFRGKAWDWEVDSENGNYDYLMYADVDMDHPEVVNELRRWGEWYTNTLNLDGFRIDAVKHIKYSFTRDWLTHVRNATGKEMFAVAEFWKNDLGALENYLNKTNWNHSVFDVPLHYNLYNASNSGGNYDMAKLLNGTVVQKHPMHAVTFVDNHDSQPGESLESFVQEWFKPLAYALILTREQGYPSVFYGDYYGIPTHSVPAMKAKIDPILEARQNFAYGTQHDYFDHHNIIGWTREGNTTHPNSGLATIMSDGPGGEKWMYVGQNKAGQVWHDITGNKPGTVTINADGWANFSVNGGSVSIWVKR

SEQ ID NO: 26 - protein, 483 aa, Bacillus halmapalus
HHNGTNGTMMQYFEWHLPNDGNHWNRLRDDASNLRNRGITAIWIPPAWKGTSQNDVGYGAYDLYDLGEFNQKGTVRTKYGTRSQLESAIHALKNNGVQVYGDVVMNHKGGADATENVLAVEVNPNNRNQEISGDYTIEAYTKFDFPGRGNTYSDFKWRWYHFDGVDWDQSRQFQNRIYKFRGKAWDWEVDSEFGNYDYLMYADYDMDHPEVVNELRRWGEWYTNTLNLDGFRIDAVKHIKFSFTRDWLTHVRNATGKGMFAVAEFWKNDLGALENYLNKTNWNHSVFDVPLHYNLYNASNSRGNYDMAKLLNGTVVQKHPMHAVTFVDNHDSQPGESLESFVQEWFKPLAYALILTREQGYPSVFYGDYYGIPTHSVPAMKAKIDPILEARQNFAYGTQHDYFDHHNIIGWTREGNTTHPNSGLATIMSDGPGGEKWMYVGQNKAGQVWHDITGNKPGTVTINADGWANFSVNKGSVSIWVKR

SEQ ID NO: 27 - protein, 514 aa, Bacillus halmapalus
MKQQKRLYARLLTLLFALIFLLPHSAAAAHHNGTNGTMMQYFEWHLPNDGNHWNRLRDDASNLRNRGITAIWIPPAWKGTSQNDVGYGAYDLYDLGEFNQKGTVRTKYGTRSQLESAIHALKNNGVQVYGDVVMNHKGGADATENVLAVEVNPNNRNQEISGDYTIEAWTKFDFPGRGNTYSDFKWRWYHFDGVDWDQSRQFQNRIYKFRGDGKAWDWEVDSENGNYDYLMYADVDMDHPEVVNELRRWGEWYTNTLNLDGFRIDAVKHIKYSFTRDWLTHVRNATGKEMFAVAEFWKNDLGALENYLNKTNWNHSVFDVPLHYNLYNASNSGGNYDMAKLLNGTVVQKHPMHAVTFVDNHDSQPGESLESFVQEWFKPLAYALILTREQGYPSVFYGDYYGIPTHSVPAMKAKIDPILEARQNFAYGTQHDYFDHHNIIGWTREGNTTHPNSGLATIMSDGPGGEKWMYVGQNKAGQVWHDITGNKPGTVTINADGWANFSVNGGSVSIWVKR

SEQ ID NO: 28 - protein, 482 aa, Bacillus halmapalus
HNGTNATMMQYFEWHLPNDGNHWNRLRDDASNLRNRGITAIWIPPAWKGTSQNDVGYGAYDLYDLGEFNQKGTVRTKYGTRSQLESAIHALKNNGVQVYGDVVMNHKAGADATENVLAVEVNPNNRNQEISGDYTIEAYTKFDFPGRGNTYSDFKWRWYHFDGVDWDQSRQFQNRIYKFRGKAWDWEVDSEFGNYDYLMYADYDMDHPEVVNELRRWGEWYTNTLNLDGFRIDAVKHIKFSFTRDWLTHVRNATGKGMFAVAEFWKNDLGALENYLSKTNWNHSVFDVPLHYNLYNASNSRGNYDMAKLLNGTVVQKHPMHAVTFVDNHDSQPGESLESFVQEWFKPLAYALILTREQGYPSVFYGDYYGIPTHSVPAMKAKIDPILAARQNFAYGTQHDYFDHHNIIGWTREGNTTHPNSGLATIMSDGPGGEKWMYVGQNKAGQVWHDITGNKPGTVTINADGWANFSVNKGSVSIWVKR

SEQ ID NO: 29 - protein, 483 aa, Bacillus licheniformis [sequence omitted]

SEQ ID NO: 30 - protein, 485 aa, Bacillus halmapalus
HHNGTNGTMMQYFEWHLPNDGNHWNRLRDDASNLRNRGITAIWIPPAWKGTSQNDVGYGAYDLYDLGEFNQKGTVRTKYGTRSQLESAIHALKNNGVQVYGDVVMNHKGGADATENVLAVEVNPNNRNQEISGDYTIEAWTKFDFPGRGNTYSDFKWRWYHFDGVDWDQSRQFQNRIYKFRGDGKAWDWEVDSENGNYDYLMYADVDMDHPEVVNELRRWGEWYTNTLNLDGFRIDAVKHIKYSFTRDWLTHVRNATGKEMFAVAEFWKNDLGALENYLNKTNWNHSVFDVPLHYNLYNASNSGGNYDMAKLLNGTVVQKHPMHAVTFVDNHDSQPGESLESFVQEWFKPLAYALILTREQGYPSVFYGDYYGIPTHSVPAMKAKIDPILEARQNFAYGTQHDYFDHHNIIGWTREGNTTHPNSGLATIMSDGPGGEKWMYVGQNKAGQVWHDITGNKPGTVTINADGWANFSVNGGSVSIWVKR

SEQ ID NO: 31 - protein, 485 aa, Bacillus sp.
HHNGTNGTMMQYFEWYLPNDGNHWNRLRSDASNLKDKGITAVWIPPAWKGASQNDVGYGAYDLYDLGEFNQKGTVRTKYGTRNQLQAAVTALKSNGIQVYGDVVMNHKGGADATEWVRAVEVNPSNRNQEVSGDYTIEAWTKFDFPGRGNTHSNFKWRWYHFDGVDWDQSRQLQNRIYKFRGDGKGWDWEVDTENGNYDYLMYADIDMDHPEVVNELRNWGVWYTNTLGLDGFRIDAVKHIKYSFTRDWLTHVRNTTGKNMFAVAEFWKNDIGAIENYLSKTNWNHSVFDVPLHYNLYNASRSGGNYDMRQIFNGTVVQRHPTHAVTFVDNHDSQPEEALESFVEEWFKPLACALTLTRDQGYPSVFYGDYYGIPTHGVPAMKSKIDPILEARQKYAYGKQNDYLDHHNMIGWTREGNTAHPNSGLATIMSDGPGGNKWMYVGRNKAGQVWRDITGNRSGTVTINADGWGNFSVNGGSVSIWVNN

SEQ ID NO: 32 - protein, 481 aa, synthetic construct
  REGION 1..481: note = Artificial Sequence
VNGTLMQYFEWYTPNDGQHWKRLQNDAEHLSDIGITAVWIPPAYKGTSQADVGYGAYDLYDLGEFHQKGTVRTKYGTKGELQSAIKSLHSRDINVYGDVVINHKGGADATEDVTAVEVDPADRNRVISGEHLIKAWTHFHFPGRGSTYSDFKWHWYHFDGTDWDESRKLNRIYKFQGKAWDWEVSNENGNYDYLMYADIDYDHPDVAAEIKRWGTWYANELQLDGFRLDAVKHIKFSFLRDWVNHVREKTGKEMFTVAEYWQNDLGALENYLNKTNFNHSVFDVPLHYQFHAASTQGGGYDMRKLLNGTVVSKHPLKSVTFVDNHDTQPGQSLESTVQTWFKPLAYAFILTRESGYPQVFYGDMYGTKGDSQREIPALKHKIEPILKARKQYAYGAQHDYFDHHDIVGWTREGDSSVANSGLAALITDGPGGAKRMYVGRQNAGETWHDITGNRSEPVVINSEGWGEFHVNGGSVSIYVQR

SEQ ID NO: 33 - protein, 483 aa, Bacillus sp.
HHNGTNGTMMQYFEWYLPNDGNHWNRLNSDASNLKSKGITAVWIPPAWKGASQNDVGYGAYDLYDLGEFNQKGTVRTKYGTRSQLQAAVTSLKNNGIQVYGDVVMNHKGGADATEMVRAVEVNPNNRNQEVTGEYTIEAWTRFDFPGRGNTHSSFKWRWYHFDGVDWDQSRRLNNRIYKFRGKAWDWEVDTENGNYDYLMYADIDMDHPEVVNELRNWGVWYTNTLGLDGFRIDAVKHIKYSFTRDWINHVRSATGKNMFAVAEFWKNDLGAIENYLQKTNWNHSVFDVPLHYNLYNASKSGGNYDMRNIFNGTVVQRHPSHAVTFVDNHDSQPEEALESFVEEWFKPLAYALTLTREQGYPSVFYGDYYGIPTHGVPAMRSKIDPILEARQKYAYGPQHDYLDHPDVIGWTREGDSSHPKSGLATLITDGPGGSKRMYAGLKNAGETWYDITGNRSDTVKIGSDGWGEFHVNDGSVSIYVQK

SEQ ID NO: 34 - protein, 484 aa, Bacillus sp.
NTAPINETMMQYFEWDLPNDGTLWTKVKNEAANLSSLGITALWLPPAYKGTSQSDVGYGVYDLYDLGEFNQKGTIRTKYGTKTQYIQAIQAAKAAGMQVYADVVFNHKAGADGTEFVDAVEVDPSNRNQETSGTYQIQAWTKFDFPGRGNTYSSFKWRWYHFDGTDWDESRKLNRIYKFRSTGKAWDWEVDTENGNYDYLMFADLDMDHPEVVTELKNWGTWYVNTTNIDGFRLDAVKHIKYSFFPDWLTYVRNQTGKNLFAVGEFWSYDVNKLHNYITKTNGSMSLFDAPLHNNFYTASKSSGYFDMRYLLNNTLMKDQPSLAVTLVDNHDTQPGQSLQSWVEPWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 35 - protein, 613 aa, Bacillus sp.
MKQQKRLYARLLTLLFALIFLLPHSAAAAANTAPINETMMQYFEWDLPNDGTLWTKVKNEAANLSSLGITALWLPPAYKGTSQSDVGYGVYDLYDLGEFNQKGTIRTKYGTKTQYIQAIQAAKAAGMQVYADVVFNHKAGADGTEFVDAVEVDPSNRNQETSGTYQIQAWTKFDFPGRGNTYSSFKWRWYHFDGTDWDESRKLNRIYKFRSTGKAWDWEVDTENGNYDYLMFADLDMDHPEVVTELKNWGTWYVNTTNIDGFRLDAVKHIKYSFFPDWLTYVRNQTGKNLFAVGEFWSYDVNKLHNYITKTNGSMSLFDAPLHNNFYTASKSSGYFDMRYLLNNTLMKDQPSLAVTLVDNHDTQPGQSLQSWVEPWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAKTSNVTFTVNNATTTSGQNVYVVANIPELGNWNTANAIKMNPSSYPTWKATIALPQGKAIEFKFIKKDQAGNVIWESTSNRTYTVPFSSTGSYTASWNVP

SEQ ID NO: 36 - protein, 483 aa, Bacillus sp.
HHDGTNGTIMQYFEWNVPNDGQHWNRLHNNAQNLKNAGITAIWIPPAWKGTSQNDVGYGAYDLYDLGEFNQKGTVRTKYGTKAELERAIRSLKANGIQVYGDVVMNHKGGADFTERVQAVEVNPQNRNQEVSGTYQIEAWTGFNFPGRGNQHSSFKWRWYHFDGTDWDQSRQLANRIYKFRGKAWDWEVDTENGNYDYLMYADVDMDHPEVINELNRWGVWYANTLNLDGFRLDAVKHIKFSFMRDWLGHVRGQTGKNLFAVAEYWKNDLGALENYLSKTNWTMSAFDVPLHYNLYQASNSSGNYDMRNLLNGTLVQRHPSHAVTFVDNHDTQPGEALESFVQGWFKPLAYATILTREQGYPQVFYGDYYGIPSDGVPSYRQQIDPLLKARQQYAYGRQHDYFDHWDVIGWTREGNASHPNSGLATIMSDGPGGSKWMYVGRQKAGEVWHDMTGNRSGTVTINQDGWGHFFVNGGSVSVWVKR

SEQ ID NO: 37 - protein, 484 aa, synthetic construct
  REGION 1..484: note = Artificial Sequence
HHNGTNGTMMQYFEWHLPNDGNHWNRLRDDASNLRNRGITAIWIPPAWKGTSQNDVGYGAYDLYDLGEFNQKGTVRTKYGTRSQLESAIHALKNNGVQVYGDVVMNHKAGADGTEFVDAVEVDPSNRNQETSGTYQIQAWTKFDFPGRGNTYSSFKWRWYHFDGTDWDESRKLNRIYKFRSTGKAWDWEVDTENGNYDYLMFADLDMDHPEVVNELRRWGEWYTNTLNLDGFRIDAVKHIKYSFTRDWLTHVRNATGKEMFAVAEFWKNDLGALENYLNKTNWNHSVFDVPLHYNLYNASNSGGNYDMAKLLNGTVVQKHPMHAVTFVDNHDSQPGESLESFVQEWFKPLAYALILTREQGYPSVFYGDYYGIPTHSVPAMKAKIDPILEARQNFAYGTQHDYFDHHNIIGWTREGNTTHPNSGLATIMSDGPGGEKWMYVGQNKAGQVWHDITGNKPGTVTINADGWANFSVNGGSVSIWVKR

SEQ ID NO: 38 - protein, 483 aa, Bacillus subtilis
HHNGTNGTLMQYFEWYLPNDGNHWNRLRSDASNLKDKGISAVWIPPAWKGASQNDVGYGAYDLYDLGEFNQKGTIRTKYGTRNQLQAAVNALKSNGIQVYGDVVMNHKGGADATEMVKAVEVNPNNRNQEVSGEYTIEAWTKFDFPGRANTHSNFKWRWYHFDGVDWDQSRKLNNRIYKFRTKAWDWEVDTEFGNYDYLLYADIDMDHPEVVNELRNWGVWYTNTLGLDGFRIDAVKHIKYSFTRDWINHVRSAIGKNMFAVAEFWKNDLGAIENYLNKTNWNHSVFDVPLHFNLYYASKSGGNYDMRQIFNGTVVQKHPTHAVTFVDNHDSQPEESLESFVREWFKPLAYALTLTREQGYPSVFYGDYYGIPTHGVPAMKSKIDPILEARQKYAYGRQNDYLDHHNIIGWTREGNTAHPNSGLATIMSDGAGGNKWMFVGRNKAGQVWTDITGNKAGTVTINADGWGNFSVNGGSVSIWVNK

SEQ ID NO: 39 - protein, 485 aa, Bacillus sp.
HHNGTNGTMMQYFEWHLPNDGNHWNRLRDDAANLKSKGITAVWIPPAWKGTSQNDVGYGAYDLYDLGEFNQKGTVRTKYGTRSQLQGAVTSLKNNGIQVYGDVVMNHKGGADGTEMVNAVEVNRSNRNQEISGEYTIEAWTKFDFPGRGNTHSNFKWRWYHFDGTDWDQSRQLQNKIYKFRGTGKAWDWEVDIENGNYDYLMYADIDMDHPEVINELRNWGVWYTNTLNLDGFRIDAVKHIKYSYTRDWLTHVRNTTGKPMFAVAEFWKNDLAAIENYLNKTSWNHSVFDVPLHYNLYNASNSGGYFDMRNILNGSVVQKHPIHAVTFVDNHDSQPGEALESFVQSWFKPLAYALILTREQGYPSVFYGDYYGIPTHGVPSMKSKIDPLLQARQTYAYGTQHDYFDHHDIIGWTREGDSSHPNSGLATIMSDGPGGNKWMYVGKHKAGQVWRDITGNRSGTVTINADGWGNFTVNGGAVSVWVKQ

SEQ ID NO: 40 - protein, 485 aa, Bacillus sp.
HHDGTNGTIMQYFEWNVPNDGQHWNRLHNNAQNLKNAGITAIWIPPAWKGTSQNDVGYGAYDLYDLGEFNQKGTVRTKYGTKAELERAIRSLKANGIQVYGDVVMNHKGGADFTERVQAVEVNPQNRNQEVSGTYQIEAWTGFNFPGRGNQHSSFKWRWYHFDGTDWDQSRQLANRIYKFRGDGKAWDWEVDTENGNYDYLMYADVDMDHPEVINELNRWGVWYANTLNLDGFRLDAVKHIKFSFMRDWLGHVRGQTGKNLFAVAEYWKNDLGALENYLSKTNWTMSAFDVPLHYNLYQASNSSGNYDMRNLLNGTLVQRHPSHAVTFVDNHDTQPGEALESFVQGWFKPLAYATILTREQGYPQVFYGDYYGIPSDGVPSYRQQIDPLLKARQQYAYGRQHDYFDHWDVIGWTREGNASHPNSGLATIMSDGPGGSKWMYVGRQKAGEVWHDMTGNRSGTVTINQDGWGHFFVNGGSVSVWVKR

SEQ ID NO: 41 - protein, 485 aa, Bacillus sp.
HHNGTNGTMMQYFEWYLPNDGNHWNRLRSDASNLKDKGISAVWIPPAWKGASQNDVGYGAYDLYDLGEFNQKGTIRTKYGTRNQLQAAVNALKSNGIQVYGDVVMNHKGGADATEMVRAVEVNPNNRNQEVSGEYTIEAWTKFDFPGRGNTHSNFKWRWYHFDGVDWDQSRKLNNRIYKFRGDGKGWDWEVDTENGNYDYLMYADIDMDHPEVVNELRNWGVWYTNTLGLDGFRIDAVKHIKYSFTRDWINHVRSATGKNMFAVAEFWKNDLGAIENYLNKTNWNHSVFDVPLHYNLYNASKSGGNYDMRQIFNGTVVQRHPMHAVTFVDNHDSQPEEALESFVEEWFKPLAYALTLTREQGYPSVFYGDYYGIPTHGVPAMKSKIDPILEARQKYAYGRQNDYLDHHNIIGWTREGNTAHPNSGLATIMSDGAGGNKWMFVGRNKAGQVWTDITGNRAGTVTINADGWGNFSVNGGSVSIWVNK